(19) United States
(12) Patent Application Publication
(10) Pub. No.: US 2025/0129145 A1
(43) Pub. Date: Apr. 24, 2025

(54) BI- AND MULTIVALENT ALBUMIN BINDERS

(71) Applicants: Ablynx N.V., Ghent-Zwijnaarde (BE); Sanofi, Paris (FR)

(72) Inventors: Tom Van Bogaert, Gentbrugge (BE); Judith Verhelst, Heusden (BE)

(21) Appl. No.: 18/891,807

(22) Filed: Sep. 20, 2024

(30) Foreign Application Priority Data
Sep. 22, 2023 (EP) 23 306 589.5

(51) Int. Cl. C07K 16/18; A61K 39/00; C07K 14/765

(52) U.S. Cl. CPC C07K 16/18; C07K 14/765; A61K 2039/505; C07K 2317/35; C07K 2317/569; C07K 2317/92; C07K 2317/94; C07K 2319/00

(57) ABSTRACT

The present technology relates to bi- and multivalent albumin binders. In particular, the present technology relates to novel and improved human serum albumin binders, specifically to polypeptides that comprise (i) at least one immunoglobulin single variable domain (ISVD) which specifically binds to a serum albumin protein or at least one human serum albumin protein and (ii) at least one further moiety specifically binding to a serum albumin protein or at least one human serum albumin protein.

### REFERENCE TO AN ELECTRONIC SEQUENCE LISTING

The contents of the electronic sequence listing (A084870238US00-SEQ-JRV.xml; Size: 175,553 bytes; and Date of Creation: Sep. 20, 2024) is herein incorporated by reference in its entirety.

### FIELD OF THE TECHNOLOGY

The present technology relates to bi- and multivalent albumin binders. In particular, the present technology relates to novel and improved human serum albumin binders, specifically to polypeptides that comprise (i) at least one immunoglobulin single variable domain (ISVD) which specifically binds to a serum albumin protein or at least one human serum albumin protein and (ii) at least one further moiety specifically binding to a serum albumin protein or at least one human serum albumin protein. The technology further relates to polypeptides comprising one albumin binder and one human serum albumin (HSA) molecule and to polypeptides comprising two HSA molecules.

The technology further relates to fusion proteins comprising the polypeptides, to nucleic acids encoding such molecules or part of such molecules; to host cells comprising such nucleic acids and/or expressing or capable of expressing such polypeptides and/or fusion proteins; to compositions, and in particular to pharmaceutical compositions that comprise such polypeptides and/or fusion proteins, nucleic acids and/or host cells.

Also, the present technology relates to methods for producing such polypeptides and fusion proteins, as well as to uses of such polypeptides and/or fusion proteins for diverse applications, including but not limited to the extension of the half-life in vivo of therapeutic compounds and/or the prevention and/or treatment of a disease and/or disorder.

### TECHNOLOGICAL BACKGROUND

Peptides and proteins are two classes of molecules with attractive possibilities for therapeutic applications. However, the bottleneck for their development to clinically and commercially relevant pharmaceuticals is their short half-life in vivo, which is typically just a few minutes to hours.

The half-life of peptides and proteins in human serum is dictated by several factors, including size, charge, proteolytic sensitivity, nature of their biology, turnover rate of proteins they bind, and others. There is a relationship between the hydrodynamic radius of a peptide or protein and its serum half-life. In general terms, those peptides and proteins that have a molecular weight smaller than approximately 70 kDa are predominantly eliminated via kidney filtration and generally possess very short serum half-lives. Larger proteins may persist in circulation for several days.

Human serum albumin (HSA) and IgG, the two most abundant soluble proteins present in blood circulation, are an exception to most proteins in circulation in that they share the remarkable property of having a prolonged serum half-life of about 19 to 21 days in human.

HSA is the most abundant plasma protein in the blood and is a carrier protein involved in many processes that serve to maintain homeostasis in the body, i.e., maintaining the oncotic pressure. Albumins are widely used as drug delivery vehicles due to their high serum concentration, their long half-life, non-toxicity and low immunogenicity and their uptake in benign and tissues, and their ability to bind to a wide variety of drugs (Mishra, V.; Heath, RJ.

Structural and Biochemical Features of Human Serum Albumin Essential for Eukaryotic Cell Culture. Int. J. Mol. Sci. 2021, 22, 8411).

This prolonged half-life of serum albumin is primarily due to it being protected from intracellular lysosomal degradation by binding to the neonatal Fc receptor (FcRn). FcRn is a heterodimer consisting of an N-glycosylated transmembrane MHC class I-like heavy chain that is noncovalently associated with soluble b2-microglobulin. Both IgG and albumin are ligands binding to different epitopes of FcRn. In general, the FcRn recycling mechanism is strictly pH-dependent and binding to FcRn is favoured at low pH (e.g., acidic endosomal pH, which is typically below 6.5) following acidification of the endosomal compartment. When albumin binds to FcRn, it escapes degradation in the lysosome. On return to the cell surface, at extracellular physiological pH (which is typically around pH 7.4), the binding is weakened, resulting in the release of albumin intothe bloodstream (see, e.g., Ward E S, Ober R J., Targeting FcRn to Generate Antibody-Based Therapeutics. Trends Pharmacol Sci., 2018; 39(10):892-904 and Andersen et al., Extending Serum Half-life of Albumin by Engineering Neonatal Fc Receptor (FcRn) Binding, J B C, 2014, 289, 19: 13492-13502).

In particular, albumin is increasingly being used to improve the pharmacokinetics of short-lived small molecule drugs that are able to bind to albumin and also to bioactive therapeutic peptides and proteins by genetic fusion of such molecules to the N- or C-terminal end of albumin (Nilsen, J., Trabjerg, E., Grevys, A. et al. An intact C-terminal end of albumin is required for its long half-life in humans. Commun Biol, 2020, 3, 181). For instance, immunoglobulin variable domain sequences (ISVDs) that can bind to serum albumin have been developed and their coupling to therapeutic compounds, moieties, and entities in order to extend the serum half-life (as defined in these applications) was described for example in WO 2004/041865, WO 2006/122787, WO 2012/175400, WO 2015/173325 and PCT/EP2016/077973. For example, WO 2006/122787 discloses as SEQ ID NO: 62 a humanized serum albumin-binding Nanobody called Alb-8 (see SEQ ID NO: 5 herein). WO 2012/175400 discloses as SEQ ID NO: 6 a humanized serum albumin-binding Nanobody called Alb-23D. Some other references that disclose ISVDs against serum albumin include WO 2003/035694, WO 2004/003019, EP 2 139 918, WO 2011/006915 and WO 2014/111550.

Other albumin binding proteins (ABP) such as albumin-binding DARPins (Designed Ankyrin Repeat Proteins) or Affitins (also known as Nanofitins) have also been described as scaffolds to extend half-life of biologics (see, e.g., Michot N. et al., “Albumin binding Nanofitins, a new scaffold to extend half-life of biologics—a case study with exenatide peptide”, Peptides, 2022, 152:170760 or Steiner D., et al., “Half-life extension using serum albumin-binding DARPin® domains”, Protein Eng Des Sel, 2017, 30(9):583-591).

However, up to now, extending the in vivo half-life of therapeutic peptides and small proteins up to or beyond the half-life of albumin or full-length antibodies has not been achieved. For instance, even though serum albumin has a half-life in humans of about 19 days, the half-life of for example albiglutide (GLP-1-HSA fusion protein Tanzeum® (US) or Eperzan® (EU) also known as Albugon)—a drug genetically fused to the N-terminal of serum albumin—is only about 5 days. Thus far, other fusion partners tested in the clinic, such as (C-terminal peptide) CTP or (Elastin-like polypeptides (ELPs) have done no better, with the fusion proteins possessing half-life values of 2.5 or 4-5 days, respectively. For most of these fusion proteins, the best dosing schedule to be expected would be weekly, with some potentially requiring two doses per week. While this is better than the native peptides or proteins alone, it is still far more frequent dosing than that of most therapeutic antibodies. At present, almost all protein-based drug formulations available in market are administered intravenously or subcutaneously with high dosing at frequent interval, eventually creating dose-fluctuation-related complications and reducing patient compliance vastly.

Accordingly, there is a need for extended serum persistence for peptide and protein-based therapeutics resulting in a more even serum concentration of the drug, lower dosage without compromising efficacy and lower dosing frequency. This may well translate into less toxicity and side effects, as well as improved compliance.

### SUMMARY OF THE TECHNOLOGY

The present technology thus provides serum albumin binders that have improved properties compared to the serum albumin binders known in the art.

The present inventors have identified albumin binding polypeptides that comprises (i) at least one immunoglobulin single variable domain (ISVD) which specifically binds to a serum albumin protein, or at least one human serum albumin protein; and (ii) at least one further moiety specifically binding to a serum albumin protein or at least one human serum albumin protein, which shows improved properties, in particular in terms of increasing the half-life and thus the efficacy of therapeutics. In a preferred embodiment, the albumin binding polypeptides of the technology comprise (i) at least one immunoglobulin single variable domain (ISVD) which specifically binds to a serum albumin protein; and (ii) at least one further moiety specifically binding to a serum albumin protein, or at least one human serum albumin protein. In a more preferred embodiment, the albumin binding polypeptides of the technology comprise (i) at least one immunoglobulin single variable domain (ISVD) which specifically binds to a serum albumin protein; and (ii) at least one further moiety specifically binding to a serum albumin protein, such as an albumin-binding ISVD, an albumin-biding DARPIN, an albumin-binding affitin or an albumin-binding domain (ABD). Even more preferably, the albumin binding polypeptides of the technology comprise (i) at least one ISVD which specifically binds to a serum albumin protein; and (ii) at least one further albumin-binding ISVD.

The albumin binding polypeptides as provided by the present technology have the advantage of showing a significantly increased in vivo serum half-life as compared to known half-life extending peptides and proteins, including full length immunoglobulins, as described in the prior art. For instance, the in vivo serum half-life of constructs comprising a single albumin binding domain, such as an albumin binding ISVD, is in the range of 0.5 to 1.4 days, see, e.g., Table 2 of Hoefman, S. et al., “Pre-clinical intravenous serum pharmacokinetics of albumin binding and non-half-life extended Nanobodies”, Antibodies, 2015, 4, 141-156.

Accordingly, the polypeptides with an extended in vivo persistence in blood circulation according to the present technology can be used for various applications, including but not limited to prolonging the in vivo half-life of (existing or future) therapeutic compounds. The benefits of extending the half-life of a therapeutic molecule will be readily apparent to those skilled in the art. Such benefits include lower doses and/or lower frequencies of administration, which reduce the risk of adverse events in the subject and reduce costs. Accordingly, therapeutics with extended half-life have a substantial added value as regards pharmaceutical significance.

In a first aspect the present technology thus provides a polypeptide that comprises (i) at least one immunoglobulin single variable domain (ISVD) which specifically binds to a serum albumin protein; and (ii) at least one further moiety specifically binding to a serum albumin protein or at least one human serum albumin protein, wherein said (i) at least one ISVD comprises three complementarity determining regions (CDR1 to CDR3, respectively), wherein:
a) CDR1 comprises the amino acid sequence of SEQ ID NO: 1 or has 3, 2 or 1 amino acid difference(s) with SEQ ID NO: 1 or CDR1 comprises the amino acid sequence of SEQ ID NO: 22 or has 3, 2 or 1 amino acid difference(s) with SEQ ID NO: 22
b) CDR2 comprises the amino acid sequence of SEQ ID NO: 2 or has 3, 2 or 1 amino acid difference(s) with SEQ ID NO: 2; and
c) CDR3 comprises the amino acid sequence of SEQ ID NO: 3 or has 3, 2 or 1 amino acid difference(s) with SEQ ID NO: 3,
   and/or
a) CDR1 comprises the amino acid sequence of SEQ ID NO: 36 or has 3, 2 or 1 amino acid difference(s) with SEQ ID NO: 36;
b) CDR2 comprises the amino acid sequence of SEQ ID NO: 37 or has 3, 2 or 1 amino acid difference(s) with SEQ ID NO: 37; and
c) CDR3 comprises the amino acid sequence of SEQ ID NO: 38 or has 3, 2 or 1 amino acid difference(s) with SEQ ID NO: 38,
   and/or
a) CDR1 comprises the amino acid sequence of SEQ ID NO: 55 or has 3, 2 or 1 amino acid difference(s) with SEQ ID NO: 55;
b) CDR2 comprises the amino acid sequence of SEQ ID NO: 56 or has 3, 2 or 1 amino acid difference(s) with SEQ ID NO: 56; and
c) CDR3 comprises the amino acid sequence of SEQ ID NO: 57 or has 3, 2 or 1 amino acid difference(s) with SEQ ID NO: 57,
   wherein the CDR sequences are determined according to AbM numbering.

The (i) at least one ISVD comprised in the polypeptide of the present technology essentially consists of 4 framework regions (FR1 to FR4, respectively) and three complementarity determining regions (CDR1 to CDR3, respectively), wherein the CDRs are as defined herein. Of course, the further ISVD(s) that may be comprised in the polypeptide and/or fusion protein of the present technology also essentially consist(s) of 4 framework regions (FR1 to FR4, respectively) and three complementarity determining regions (CDR1 to CDR3, respectively).

The present technology also provides a fusion protein or construct comprising the polypeptide of the present technology and one or more (such as one or two, or more) other amino acid sequences, (binding) domains, binding units or other moieties or chemical entities. The fusion protein may preferably comprise the polypeptide of the technology, at least one further albumin-binding domain and/or at least one therapeutic and/or targeting moiety. The present technology also provides the polypeptide, fusion protein and/or composition of the technology for use in medicine (as a medicament).

Provided herewith is also a composition comprising the polypeptide and/or the fusion protein of the present technology, as well as methods for producing the same, nucleic acid sequences encoding the same, vectors comprising the nucleic acid sequences and host cells comprising the nucleic acids and/or the vectors of the present technology.

Finally, the present technology provides a kit comprising the polypeptide, the fusion protein, the nucleic acid or nucleic acid sequence, the vector or the host cell according of the present technology.

### BRIEF DESCRIPTION OF THE FIGURES

FIG. 1. Schematic representation of the constructs (fusion proteins or polypeptides) of Examples 1 and 2. “ALB” refers to albumin binding ISVD building block (ALB23002, SEQ ID NO.: 4); “X”, “Y” and “Z” refer to possible future therapeutic leads. “CNB” refers to irrelevant or control ISVD building blocks (CNB or IRR) not binding to serum albumin or any other envisaged target, but solely included in the polypeptide construct so as to create a similar size.

FIG. 2. Mean (+/−SD, n=4 to 6) serum concentration-time profiles of test fusion proteins, following i.v. bolus administration at equimolar amounts (3.5 mg/kg for tetravalent constructs or 2.7 mg/kg for trivalent construct in Tg32 mice).

FIG. 3. Schematic representation of polypeptides and fusion proteins (constructs) according to the technology and used in the examples.

### DETAILED DESCRIPTION OF THE TECHNOLOGY

### Definitions

Unless indicated or defined otherwise, all terms used have their usual meaning in the art, which will be clear to the skilled person. Reference is, for example, made to the standard handbooks, such as Sambrook et al., 1989 (Molecular Cloning: A Laboratory Manual, 2^{nd} Ed., Vols. 1-3, Cold Spring Harbor Laboratory Press), Ausubel et al., 1987 (Current protocols in molecular biology, Green Publishing and Wiley Interscience, New York), Lewin 1985 (Genes II, John Wiley & Sons, New York, N.Y.), Old et al., 1981 (Principles of Gene Manipulation: An Introduction to Genetic Engineering, 2^{nd} Ed., University of California Press, Berkeley, CA), Roitt et al., 2001 (immunology, 6^{th} Ed., Mosby/Elsevier, Edinburgh), Roitt et al., 2001 (Roitt's Essential Immunology, 10^{th} Ed., Blackwell Publishing, UK), and Janeway et al., 2005 (Immunobiology, 6^{th} Ed., Garland Science Publishing/Churchill Livingstone, New York), as well as to the general background art cited herein.

Unless indicated otherwise, all methods, steps, techniques and manipulations that are not specifically described in detail herein can be performed and have been performed in a manner known per se, as will be clear to the skilled person. Reference is, for example, again made to the standard handbooks and the general background art mentioned herein and to the further references cited therein; as well as to for example the following reviews: Presta 2006 (Adv. Drug Deliv. Rev., 58: 640), Levin and Weiss 2006 (Mol. Biosyst., 2: 49), Irving et al., 2001 (J. Immunol. Methods, 248: 31), Schmitz et al., 2000 (Placenta 21 Suppl. A: S106), Gonzales et al., 2005 (Tumour Biol., 26: 31), which describe techniques for protein engineering, such as affinity maturation and other techniques for improving the specificity and other desired properties of proteins such as immunoglobulins.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize or be able to ascertain using no more than routine experimentation, many equivalents to the specific embodiments of the technology described herein. Such equivalents are intended to be encompassed by the present technology.

The term “and/or” wherever used herein includes the meaning of “and”, “or” and “all or any other combination of the elements connected by said term.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

“Similar”, as used herein, is interchangeable for alike, analogous, comparable, corresponding, and -like, and is meant to have the same or common characteristics, and/or in a quantifiable manner to show comparable results i.e., with a variation of maximum 20%, 10%, more preferably 5%, or even more preferably 1%, or less.

The term “sequence” as used herein (for example in terms like “immunoglobulin sequence”, “antibody sequence”, “variable domain sequence”, “V_{HH} sequence”, “ISVD sequence” or “protein sequence”), should generally be understood to include both the relevant amino acid sequence as well as nucleic acids or nucleotide sequences encoding the same, unless the context requires a more limited interpretation. Amino acid sequences are interpreted to mean a single amino acid or an unbranched sequence of two or more amino acids, depending on the context. Nucleotide sequences are interpreted to mean an unbranched sequence of 3 or more nucleotides.

When a nucleotide sequence or amino acid sequence is said to “comprise” another nucleotide sequence or amino acid sequence, respectively, or to “essentially consist of” another nucleotide sequence or amino acid sequence, this may mean that the latter nucleotide sequence or amino acid sequence has been incorporated into the first mentioned nucleotide sequence or amino acid sequence, respectively, but more usually this generally means that the first mentioned nucleotide sequence or amino acid sequence comprises within its sequence a stretch of nucleotides or amino acid residues, respectively, that has the same nucleotide sequence or amino acid sequence, respectively, as the latter sequence, irrespective of how the first mentioned sequence has actually been generated or obtained (which may for example be by any suitable method described herein). By means of a non-limiting example, when an ISVD is said to comprise a CDR sequence, this may mean that said CDR sequence has been incorporated into the ISVD, but more usually this generally means that the ISVD contains within its sequence a stretch of amino acid residues with the same amino acid sequence as said CDR sequence, irrespective of how said ISVD has been generated or obtained. It should also be noted that when the latter amino acid sequence has a specific biological or structural function, it preferably has essentially the same, a similar or an equivalent biological or structural function in the first-mentioned amino acid sequence (in other words, the first-mentioned amino acid sequence is preferably such that the latter sequence is capable of performing essentially the same, a similar or an equivalent biological or structural function).

For example, when an ISVD is said to comprise a CDR sequence or framework sequence, respectively, the CDR sequence and framework are preferably capable, in said ISVD, of functioning as a CDR sequence or framework sequence, respectively. Also, when a nucleotide sequence is said to comprise another nucleotide sequence, the first-mentioned nucleotide sequence is preferably such that, when it is expressed into an expression product (e.g., a polypeptide), the amino acid sequence encoded by the latter nucleotide sequence forms part of said expression product (in other words, that the latter nucleotide sequence is in the same reading frame as the first-mentioned, larger nucleotide sequence).

Amino acids are organic compounds that contain amino[a] (—NH⁺₃) and carboxylate (—CO⁻₂) functional groups, along with a side chain (R group) specific to each amino acid-Amino acid residues will be indicated interchangeably herein according to the standard three-letter or one-letter amino acid code, as mentioned in Table 1 below.

TABLE 1
Common amino acids

| 1-Letter | 3-Letter | Amino |
|---|---|---|
| Code | Code | Acid Name |
| A | Ala | Alanine |
| C | Cys | Cysteine |
| D | Asp | Aspartic acid |
| E | Glu | Glutamic acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| X | Xaa | Unspecified |
| Y | Tyr | Tyrosine |

When an amino acid residue is indicated as “X” or “Xaa”, it means that the amino acid residue is unspecified, unless the context requires a more limited interpretation. For example, if the description provides an amino acid sequence of a CDR wherein one (or more) of the amino acid residue(s) is (are) indicated with “X”, the description may further specify which amino acid residue(s) is (can be) present at that specific position of the CDR.

Amino acids are those amino acids commonly found in naturally occurring proteins and are listed in Table 1. Any amino acid sequence that contains post-translationally modified amino acids may be described as the amino acid sequence that is initially translated usingthe symbols shown in the Table 1 with the modified positions, e.g., hydroxylations or glycosylations, but these modifications shall not be shown explicitly in the amino acid sequence. Any peptide or protein that can be expressed as a sequence modified linkages, cross links and end caps, non-peptidyl bonds, etc., is embraced by this definition. The terms “protein”, “peptide”, “protein/peptide”, and “polypeptide” are used interchangeably throughout the disclosure, and each has the same meaning for purposes of this disclosure. Each term refers to an organic compound made of a linear chain of two or more amino acids. The compound may have ten or more amino acids; twenty-five or more amino acids; fifty or more amino acids; one hundred or more amino acids, two hundred or more amino acids, and even three hundred or more amino acids. The skilled artisan will appreciate that polypeptides generally comprise fewer amino acids than proteins, although there is no art-recognized cut-off point of the number of amino acids that distinguish a polypeptide from a protein; those polypeptides may be made by chemical synthesis or recombinant methods; and those proteins are generally made in vitro or in vivo by recombinant methods as known in the art.

By convention, the amide bond in the primary structure of polypeptides is in the order that the amino acids are written, in which the amine end (N-terminus) of a polypeptide is always on the left, while the acid end (C-terminus) is on the right.

Any amino acid sequence that contains post-translationally modified amino acids may be described as the amino acid sequence that is initially translated using the symbols shown in Table 1 with the modified positions; e.g., hydroxylations or glycosylations, but these modifications shall not be shown explicitly in the amino acid sequence. Any peptide or protein that can be expressed as a sequence modified linkages, cross links and end caps, non-peptidyl bonds, etc., is embraced by this definition.

The term “domain” as used herein generally refers to a globular region of an antibody chain, and in particular to a globular region of a heavy chain antibody, or to a polypeptide that essentially consists of such a globular region. Usually, such a domain will comprise peptide loops (for example 3 or 4 peptide loops) stabilized, for example, as a sheet or by disulfide bonds.

The term “about” used in the context of the parameters or parameter ranges of the provided herein shall have the following meanings. Unless indicated otherwise, where the term “about” is applied to a particular value or to a range, the value or range is interpreted as being as accurate as the method used to measure it. If no error margins are specified in the application, the last decimal place of a numerical value indicates its degree of accuracy. Where no other error margins are given, the maximum margin is ascertained by applying the rounding-off convention to the last decimal place, e.g., for a pH value of about pH 2.7, the error margin is 2.65-2.74. In the present application, any parameter indicated with the term “about” is also contemplated as being disclosed without the term “about”. In other words, embodiments referring to a parameter value using the term “about” shall also describe an embodiment directed to the numerical value of said parameter as such. For example, an embodiment specifying a pH of “about pH 2.7” shall also disclose an embodiment specifying a pH of “pH 2.7” as such; an embodiment specifying a pH range of “between about pH 2.7 and about pH 2.1” shall also describe an embodiment specifying a pH range of “between pH 2.7 and pH 2.1”, etc.

For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position). Alternatively, the degree of sequence identity between two or more nucleotide sequences may be calculated using a known computer algorithm for sequence alignment such as NCBI Blast v2.0, using standard settings. Some other techniques, computer algorithms and settings for determining the degree of sequence identity are for example described in WO 04/037999, EP 0967284, EP 1085089, WO 00/55318, WO 00/78972, WO 98/49185 and GB 2357768. Usually, for the purpose of determining the percentage of “sequence identity” between two nucleotide sequences in accordance with the calculation method outlined hereinabove, the nucleotide sequence with the greatest number of nucleotides will be taken as the “first” nucleotide sequence, and the other nucleotide sequence will be taken as the “second” nucleotide sequence.

For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e., as an “amino acid difference” as defined herein. Alternatively, the degree of sequence identity between two amino acid sequences may be calculated using a known computer algorithm, such as those mentioned above for determining the degree of sequence identity for nucleotide sequences, again using standard settings. Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure, and which has little or essentially no influence on the 3D structure, function, activity, or other biological properties of the polypeptide. Such conservative amino acid substitutions are well known in the art, for example from WO 04/037999, GB 335768, WO 98/49185, WO 00/46383, and WO 01/09300; and (preferred) types and/or combinations of such substitutions may be selected on the basis of the pertinent teachings from WO 04/037999 as well as WO 98/49185 and from the further references cited therein.

Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gin; (c) polar, positively charged residues: His, Arg and Lys; (d) large aliphatic, nonpolar residues: Met, Leu, lie, Val and Cys; and (e) aromatic residues: Phe, Tyr and Trp. Particularly preferred conservative substitutions are as follows: Ala into Gly or into Ser; Arg into Lys; Asn into Gin or into His; Asp into Glu; Cys into Ser; Gin into Asn; Glu into Asp; Gly into Ala or into Pro; His into Asn or into Gin; lie into Leu or into Val; Leu into lie or into Val; Lys into Arg, into Gin or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu.

When comparing two immunoglobulin single variable domains, the term “amino acid difference” refers to an insertion, deletion or substitution of a single amino acid residue on a position of the first sequence, compared to the second sequence; it being understood that two immunoglobulin single variable domains can contain one, two or more such amino acid differences.

For the purposes of comparing two or more immunoglobulin single variable domains or other amino acid sequences such as, e.g., the polypeptides of the technology, etc., the percentage of “sequence identity” between a first amino acid sequence and a second amino acid sequence (also referred to herein as “amino acid identity”) may be calculated or determined as described in paragraph f) on pages 49 and 50 of WO 08/020079 (incorporated herein by reference), such as by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e., as an “amino acid difference” as defined herein; alternatively, the degree of sequence identity between two amino acid sequences may be calculated using a known computer algorithm for sequence alignment, such as NCBI Blast v2.0, using standard settings. Some other techniques, computer algorithms and settings for determining the degree of sequence identity are for example described in WO 04/037999, EP 0 967 284, EP 1 085 089, WO 00/55318, WO 00/78972, WO 98/49185 and GB 2 357 768-A.

Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Also, in determining the degree of sequence identity between two immunoglobulin single variable domains, the skilled person may take into account so-called “conservative” amino acid substitutions, which can generally be described as amino acid substitutions in which an amino acid residue is replaced with another amino acid residue of similar chemical structure and which has little or essentially no influence on the function, activity, or other biological properties of the polypeptide. Such conservative amino acid substitutions are well known in the art, for example from WO 04/037999, GB-A-3 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein. Examples of conservative substitutions are described herein further below.

Any amino acid substitutions applied to the polypeptides described herein may also be based on the analysis of the frequencies of amino acid variations between homologous proteins of different species developed by Schulz et al., 1978 (Principles of Protein Structure, Springer-Verlag), on the analyses of structure forming potentials developed by Chou and Fasman 1975 (Biochemistry 13: 211) and 1978 (Adv. Enzymol. 47: 45-149), and on the analysis of hydrophobicity patterns in proteins developed by Eisenberg et al., 1984 (Proc. Natl. Acad. Sci. USA 81: 140-144), Kyte & Doolittle 1981(J Molec. Biol. 157: 105-132), and Goldman et al. 1986 (Ann. Rev. Biophys. Chem. 15: 321-353), all incorporated herein in their entirety by reference. Information on the primary, secondary and tertiary structure of ISVDs is given in the description herein and in the general background art cited above. Also, for this purpose, the crystal structure of a V_{HH} domain from a llama is for example given by Desmyter et al., 1996 (Nature Structural Biology, 3: 803), Spinelli et al., 1996 (Natural Structural Biology 3: 752-757), and Decanniere et al., 1999 (Structure, 7: 361). Further information about some of the amino acid residues that in conventional V_{H} domains form the V_{H}/V_{L} interface and potential camelizing substitutions on these positions can be found in the prior art cited above.

Immunoglobulin single variable domains and nucleic acid sequences are said to be “exactly the same” if they have 100% sequence identity (as defined herein) over their entire length.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene orgene product that displays modifications in sequence, post-translational modifications and/or functional properties (i.e., altered characteristics) when compared to the wild-type gene or gene product. It is noted that naturally occurring mutants can be isolated; these are identified by the fact that they have altered characteristics when compared to the wild-type gene or gene product. Alternatively, a variant may also include synthetic molecules, e.g., a chemokine ligand variant may be similar in structure and/or function to the natural chemokine, but may concern a small molecule, or a synthetic peptide or protein, which is man-made. Said variants with different functional properties may concerns super-agonists, super antagonists, among other functional differences, as known to the skilled person.

In the context of the present technology, the terms “specificity”, “binding specifically” or “specific binding” refer to the number of different target molecules, such as antigens, to which a particular binding unit can bind with sufficiently high affinity (see below). “Specificity”, “binding specifically” or “specific binding” are used interchangeably herein with “selectivity”, “binding selectively” or “selective binding”. Generally, binding units, such as binding ISVDs, specifically bind to their designated targets.

The specificity/selectivity of a binding unit can be determined based on affinity. The affinity denotes the strength or stability of a molecular interaction. The affinity is commonly given by the K_{D}, or dissociation constant, which has units of mol/litre (or M). The affinity can also be expressed as an association constant, K_{A}, which equals 1/K_{D} and has units of (mol/litre)⁻¹ (or M⁻¹).

The affinity is a measure for the binding strength between a moiety and a binding site on a target molecule: the lower the value of the K_{D}, the stronger the binding strength between a target molecule and a targeting moiety.

The K_{D}-value characterizes the strength of a molecular interaction also in a thermodynamic sense as it is related to the change of free energy (DG) of binding by the well-known relation DG=RT·ln(K_{D}) (equivalently DG=−RT·ln(K_{A})), where R equals the gas constant, T equals the absolute temperature and In denotes the natural logarithm.

The K_{D} may also be expressed as the ratio of the dissociation rate constant of a complex, denoted as k_{off}, to the rate of its association, denoted kₒₙ (so that K_{D}=k_{off}/kₒₙ and K_{A}=kₒₙ/k_{off}). The off-rate k_{off} has units s⁻¹ (where s is the SI unit notation of second). The on-rate kₒₙ has units M⁻¹s⁻¹. The on-rate may vary between 10² M⁻¹s⁻¹ to about 10¹ M⁻¹s⁻¹, approaching the diffusion-limited association rate constant for bimolecular interactions. The off-rate is related to the half-life of a given molecular interaction by the relation t_{1/2}=ln(2)/k_{off}. The off-rate may vary between 10⁻⁶s⁻¹ (near irreversible complex with a tin of multiple days) to 1 s⁻¹ (t_{1/2}=0.69 s).

The measured K_{D} may correspond to the apparent K_{D} if the measuring process somehow influences the intrinsic binding affinity of the implied molecules for example by artefacts related to the coating on the biosensor of one molecule. Also, an apparent K_{D} may be measured if one molecule contains more than one recognition sites for the other molecule or molecules. In such situation the measured affinity may be affected by the avidity of the interaction by the two molecules.

The dissociation constant (K_{D}) may be the actual or apparent dissociation constant, as will be clear to the skilled person. Methods for determining the K_{D} will be clear to the skilled person, and for example include the techniques mentioned below. In this respect, it will also be clear that it may not be possible to measure dissociation constants of more than 10⁻⁴ moles/litre or 10⁻³ moles/litre (e.g., of 10⁻² moles/litre). Optionally, as will also be clear to the skilled person, the (actual or apparent) K_{D} may be calculated on the basis of the (actual or apparent) association constant (K_{A}), by means of the relationship (K_{D}=1/K_{A}). K_{A}=1/K_{D}-->K_{A}=[AB]/[A]·[B].

In the context of the present technology, the term “avidity” refers to the accumulated strength of multiple affinities of individual non-covalent binding interactions, such as between a protein receptor and its ligand, and may also be referred to as “functional affinity”. “Avidity” differs from “affinity”, which, as explained in detail above, describes the strength of a single interaction.

As used herein, the term “binding site” comprises a region of a polypeptide which is responsible for selectively binding to a target antigen of interest (e.g., aChR). Binding domains comprise at least one binding site. Exemplary binding domains include an antibody variable domain. Antibody molecules may comprise a single binding site or multiple (e.g., two, three or four) binding sites.

The terms “variable region” and “variable domain” are used herein interchangeable and are intended to have equivalent meaning. The term “variable” refers to the fact that certain portions of the variable domains VH and VL differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the VL domain and the VH domain which form part of the antigen binding site.

In the context of the present technology, “acidic pH” or “acidic endosomal pH” refers to an acid physiological pH, such as the pH inside endosomes, which is generally pH<6.8, or <6.5, such as between 5.0 and 6.8, such as about 5.1, 5.2, 5.3, 5.4, 5.5, 5.6, 5.7, 5.8, 5.9, 6.0, 6.1, 6.2, 6.3, 6.4, 6.5, 6.6, or 6.7, or such as about pH 6.4, 6.3, 6.2, 6.1 or about pH 6.0. Hence, “acidic pH” may refer to a pH of between about 6.0 and about 6.5. In the context of the present technology, “neutral pH”, “near neutral pH” or “extracellular physiological pH” refers to the pH of the extracellular space, e.g., a pH of about 7.0 to about 7.5, such as about 7.0, 7.1, 7.2, 7.3, 7.4 or 7.5, preferably about 7.4.

### The Polypeptide of the Present Technology

The present inventors have developed novel polypeptides comprising (i) at least one immunoglobulin single variable domain (ISVD) which specifically binds to a serum albumin protein or at least one human serum albumin protein; and (ii) at least one further moiety specifically binding to a serum albumin protein or at least one human serum albumin protein.

In a preferred embodiment, the polypeptides of the present technology comprise (i) at least one immunoglobulin single variable domain (ISVD) which specifically binds to a serum albumin protein; and (ii) at least one further moiety specifically binding to a serum albumin protein or at least one human serum albumin protein.

In a more preferred embodiment, the albumin binding polypeptides of the technology comprise (i) at least one ISVD which specifically binds to a serum albumin protein; and (ii) at least one further moiety specifically binding to a serum albumin protein, such as an albumin-binding ISVD, an albumin-binding DARPIN, an albumin-binding affitin or an albumin-binding domain (ABD). Even more preferably, the albumin binding polypeptides of the technology comprise (i) at least one ISVD which specifically binds to a serum albumin protein; and (ii) at least one further albumin-binding ISVD.

First Domain: At Least One ISVD which Specifically Binds to a Serum Albumin Protein or at Least One Human Serum Albumin Protein

In the context of the present technology, the term “immunoglobulin single variable domain” (ISVD), interchangeably used with “single variable domain”, defines immunoglobulin molecules wherein the antigen binding site is present on, and formed by, a single immunoglobulin domain. This sets ISVDs apart from “conventional” immunoglobulins (e.g., monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e., a total of 6 CDRs will be involved in antigen binding site formation.

In view of the above definition, the antigen-binding domain of a conventional 4-chain antibody (such as an IgG, IgM, IgA, IgD or IgE molecule; known in the art) or of a Fab fragment, a F(ab′)₂ fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, ISVDs are capable of specifically binding to an epitope of the antigen without pairing with an additional immunoglobulin variable domain. The binding site of an immunoglobulin single variable domain is formed by a single V_{H}, a single V_{HH} or single V_{L} domain.

In the context of the present technology, the single variable domain may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An ISVD can for example be a heavy chain ISVD, such as a V_{H}, V_{HH}, including a camelized V_{H} or humanized V_{HH}. Preferably, it is a V_{HH}, including a camelized V_{H} or humanized V_{HH}. Heavy chain ISVDs can be derived from a conventional four-chain antibody or from a heavy chain antibody.

For example, the ISVD may be a single domain antibody (or an amino acid sequence that is suitable for use as a single domain antibody), a “dAb” or dAb (or an amino acid sequence that is suitable for use as a dAb) or a Nanobody ISVD (as defined herein and including but not limited to a V_{HH}); other single variable domains, or any suitable fragment of any one thereof. Preferably, the ISVD is a V_{H}, a humanized V_{H}, a human V_{H}, a V_{HH}, a humanized V_{HH} or a camelized V_{H}. More preferably, the ISVD is a Nanobody ISVD (such as a V_{HH}, including a humanized V_{HH} or camelized V_{H}) or a suitable fragment thereof. Nanobody® is a registered trademark from Ablynx N.V.

“V_{HH} domains”, also known as V_{HH}s, V_{HH} antibody fragments, and V_{HH} antibodies, have originally been described as the antigen binding immunoglobulin variable domain of “heavy chain antibodies” (i.e., of “antibodies devoid of light chains”; Hamers-Casterman et ol., Nature 363: 446-448, 1993). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}s, reference is made to the review article by Muyldermans et al., 2001 (Reviews in Molecular Biotechnology 74: 277-302, 2001). V_{HH} domains can be obtained from heavy chain-only antibodies (HCAbs) that are circulating in Camelidae, see e.g., Muyldermans S., “A guide to: generation and design of nanobodies”, FEBS J., 2021, 288(7):2084-2102.

Typically, the generation of immunoglobulins involves the immunization of experimental animals, fusion of immunoglobulin producing cells to create hybridomas and screening for the desired specificities. Alternatively, immunoglobulins can be generated by screening of naïve or synthetic libraries, e.g., by phage display.

The generation of immunoglobulin sequences, such as Nanobody V_{HH}s, has been described extensively in various publications, among which WO 94/04678, Hamers-Casterman et al., 1993 and Muyldermans et al., 2001 (Reviews in Molecular Biotechnology 74: 277-302, 2001) can be exemplified. In these methods, camelids are immunized with the target antigen in order to induce an immune response against said target antigen. The repertoire of V_{HH}s obtained from said immunization is further screened for V_{HH}S that bind the target antigen.

In these instances, the generation of antibodies requires purified antigen for immunization and/or screening. Antigens can be purified from natural sources, or in the course of recombinant production.

Immunization and/or screening for immunoglobulin sequences can be performed using peptide fragments of such antigens. The present technology may use immunoglobulin sequences of different origin, comprising mouse, rat, rabbit, donkey, human and camelid immunoglobulin sequences. The technology also includes fully human, humanized or chimeric sequences. For example, the technology comprises camelid immunoglobulin sequences and humanized camelid immunoglobulin sequences, or camelized domain antibodies, e.g., camelized dAb as described by Ward et al. (see for example WO 94/04678 and Riechmann, Febs Lett., 339:285-290, 1994 and Prot. Eng., 9:531-537, 1996). Moreover, the technology also uses fused immunoglobulin sequences, e.g. forming a multivalent and/or multispecific construct (for multivalent and multispecific polypeptides containing one or more V_{HH} domains and their preparation, reference is also made to Conrath et al., J. Biol. Chem., Vol. 276, 10. 7346-7350, 2001, as well as to for example WO 96/34103 and WO 99/23221), and immunoglobulin sequences comprising tags or other functional moieties, e.g., toxins, labels, radiochemicals, etc., which are derivable from the immunoglobulin sequences of the present technology.

A “humanized V_{HH}” comprises an amino acid sequence that corresponds to the amino acid sequence of a naturally occurring V_{HH} domain, but that has been “humanized”, i.e., by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g., indicated above). This can be performed in a manner known per se, which will be clear to the skilled person, for example on the basis of the further description herein and the prior art (e.g., WO 2008/020079). Again, it should be noted that such humanized V_{HH}S can be obtained in any suitable manner known per se and thus are not strictly limited to polypeptides that have been obtained using a polypeptide that comprises a naturally occurring V_{HH} domain as a starting material.

A “camelized V_{H}” comprises an amino acid sequence that corresponds to the amino acid sequence of a naturally occurring V_{H} domain, but that has been “camelized”, i.e., by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the further description herein and the prior art (e.g., WO 2008/020079). Such “camelizing” substitutions are preferably inserted at amino acid positions that form and/or are present at the V_{H}-V_{L} interface, and/or at the so-called Camelidae hallmark residues, as defined herein (see for example WO 94/04678 and Davies and Riechmann (1994 and 1996), supra). Preferably, the V_{H} sequence that is used as a starting material or starting point for generating or designing the camelized V_{H} is preferably a V_{H} sequence from a mammal, more preferably the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

The structure of an ISVD sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

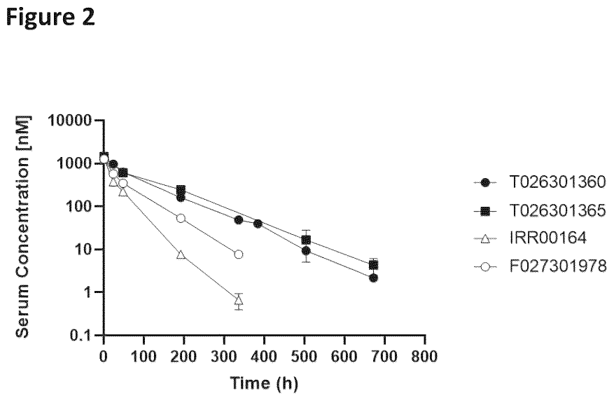

As further described in paragraph q) on pages 58 and 59 of WO 08/020079, the amino acid residues of an immunoglobulin single variable domain can be numbered according to the general numbering for V_{H} domains given by Kabat et al. (“Sequence of proteins of immunological interest”, US Public Health Services, NIH Bethesda, MD, Publication No. 91), as applied to V_{H}H domains from Camelids in the article of Riechmann and Muyldermans, 1999 (J. Immunol. Methods 231(1-2):25-38; see for example FIG. 2 of this publication). It should be noted that—as is well known in the art for V_{H} domains and for V_{HH} domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat (according to Kabat annotation or Kabat numbering) may or may not correspond to the actual numbering of the amino acid residues in the actual sequence. The total number of amino acid residues in a V_{H} domain and a V_{HH} domain will usually be in the range of from 110 to 120, often between 112 and 115. It should however be noted that smaller and longer sequences may also be suitable for the purposes described herein.

In the present application, unless indicated otherwise, CDR sequences were determined according to Kabat numbering with AbM CDR annotation (“according to AbM annotation” or “AbM numbering”) as described in Kontermann and Dubel (Eds. 2010, Antibody Engineering, vol 2, Springer Verlag Heidelberg Berlin, Martin, Chapter 3, pp. 33-51). According to this method, FR1 comprises the amino acid residues at positions 1-25, CDR1 comprises the amino acid residues at positions 26-35, FR2 comprises the amino acids at positions 36-49, CDR2 comprises the amino acid residues at positions 50-58, FR3 comprises the amino acid residues at positions 59-94, CDR3 comprises the amino acid residues at positions 95-102, and FR4 comprises the amino acid residues at positions 103-113.

Determination of CDR regions may also be done according to different methods. In the CDR determination according to Kabat, FR1 of an immunoglobulin single variable domain comprises the amino acid residues at positions 1-30, CDR1 of an immunoglobulin single variable domain comprises the amino acid residues at positions 31-35, FR2 of an immunoglobulin single variable domain comprises the amino acids at positions 36-49, CDR2 of an immunoglobulin single variable domain comprises the amino acid residues at positions 50-65, FR3 of an immunoglobulin single variable domain comprises the amino acid residues at positions 66-94, CDR3 of an immunoglobulin single variable domain comprises the amino acid residues at positions 95-102, and FR4 of an immunoglobulin single variable domain comprises the amino acid residues at positions 103-113.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are preferably (a suitable combination of) immunoglobulin framework sequences or framework sequences that have been derived from immunoglobulin framework sequences (for example, by humanization or camelization). For example, the framework sequences may be framework sequences derived from a light chain variable domain (e.g., a V_{L}-sequence) and/or from a heavy chain variable domain (e.g., a V_{H}-sequence or V_{HH} sequence). In one particularly preferred aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISVD sequence used in the technology may contain one or more of hallmark residues (as defined herein), such that the ISVD sequence is a Nanobody molecule, such as a V_{HH}, including a humanized V_{HH} or camelized V_{H}. Some preferred, but non-limiting examples of (suitable combinations of) such framework sequences will become clear from the further disclosure herein.

However, it should be noted that, in the context of the present technology, the origin of the ISVD sequence or the origin of the nucleotide sequence used to express it is not limited, nor as to the way that the ISVD sequence or nucleotide sequence is or has been generated or obtained. Thus, the ISVD sequences may be naturally occurring sequences (from any suitable species) or synthetic or semi-synthetic sequences. In a specific but non-limiting aspect, the ISVD sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences, as well as immunoglobulin sequences that have been obtained by techniques such as affinity dematuration (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g., DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

For a general description of ISVDs, reference is made to the further description below, as well as to the prior art cited herein. In this respect, it should however be noted that this description and the prior art mainly described ISVDs of the so-called “V_{H}3 class” (i.e., ISVDs with a high degree of sequence homology to human germline sequences of the V_{H}3 class such as DP-47, DP-51 or DP-29). It should however be noted that the technology in its broadest sense can generally use any type of ISVD, and for example also uses the ISVDs belonging to the so-called “V_{H}4 class” (i.e., ISVDs with a high degree of sequence homology to human germline sequences of the V_{H}4 class such as DP-78), as for example described in WO 2007/118670.

Preferably, the ISVD is derived from an ISVD belonging to the “V_{H}3 class”.

Generally, ISVDs (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, a ISVD can be defined as an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, an ISVD can be an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which the framework sequences are as further defined herein.

More in particular, an ISVD can be an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which: one or more of the amino acid residues at positions 11, 37, 44, 45, 47, 83, 84, 103, 104 and 108 according to the Kabat numbering are chosen from the Hallmark residues mentioned in Table 2 below.

TABLE 2
Hallmark Residues in Nanobody ® ISVDs

| Position | Human V_{H}3 | Hallmark Residues |
|---|---|---|
| 11 | L, V; | L, S, V, M, W, F, T, Q, E, A, R, |
| | perdominantly L | G, K, Y, N, P, I; preferably L |
| 37 | V, I, F; | F⁽¹⁾, Y, V, L, A, H, S, I, W, C, N, |
| | usually V | G, D, T, P, preferably F⁽¹⁾ or Y |
| 44⁽⁸⁾ | G | E⁽³⁾, Q⁽³⁾, G⁽²⁾, D, A, K, R, L, P, S, |
| | | V, H, T, N, W, M, I; |
| | | preferably G⁽²⁾, E⁽³⁾ or Q⁽³⁾; |
| | | most preferably G⁽²⁾ or Q⁽³⁾. |
| 45⁽⁸⁾ | L | L⁽²⁾, R⁽³⁾, P, H, F, G, Q, S, E, T, Y, C, |
| | | I, D, V; preferably L⁽²⁾ or R⁽³⁾ |
| 47⁽⁸⁾ | W, Y | F⁽¹⁾, L⁽¹⁾, or W⁽²⁾ G, I, S, A, V, M, |
| | | R, Y, E, P, Y, C, H, K, Q, N, D; |
| | | preferably W⁽²⁾, L⁽¹⁾, or F⁽¹⁾ |
| 83 | R or K; | R, K⁽⁵⁾, T, E⁽⁵⁾, Q, N, S, I, V, |
| | | G, M, L, A, D, Y, H; |
| | usually R | preferably K or R; most preferably K |
| 84 | A, T, D; | P⁽⁵⁾, S, H, L, A, V, I, T, F, D, R, |
| | predominantly A | Y, N, Q, G, E; preferably P |
| 103 | W | W⁽⁴⁾, R⁽⁶⁾, G, S, K, A, M, Y, L, F, T, N, |
| | | V, Q, P⁽⁶⁾, E, C; preferably W |
| 104 | G | G, A, S, T, D, P, N, E, C, L; preferably G |
| 108 | L, M, or T; | Q, L⁽⁷⁾, R, P, E, K, S, T, M, A, H; |
| | predominantly L | preferably Q or L⁽⁷⁾ |

Notes:
⁽¹⁾In particular, but not exclusively, in combination with KERE or KQRE at positions 43-46.
⁽²⁾Usually as GLEW at positions 44-47.
⁽³⁾Usually as KERE or KQRE at positions 43-46, e.g. as KEREL, KEREF, KQREL, KQREF, KEREG, KQREW or KQREG at positions 43-47. Alternatively, also sequences such as TERE (for example TEREL), TQRE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
⁽⁴⁾With both GLEW at positions 44-47 and KERE or KQRE at positions 43-46.
⁽⁵⁾Often as KP or EP at positions 83-84 of naturally occuring V_{HH} domains.
⁽⁶⁾In particular, but not exclusively, in combination with GLEW at positions 44-47.
⁽⁷⁾With the proviso that when positions 44-47 are GLEQ, position 108 is always Q in (non-humanized) VHH sequences that also contain a W at 103.
⁽⁸⁾The GLEW group also contains GLEW-like sequences at positions 44-47, such as for example GVEW, EPEW, GLER, DQEW, DLEW, GIEW, ELEW, GPEW, EWLP, GPER, GLER and ELEW

Thus, a Nanobody ISVD can be defined as an amino acid sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which one or more of the amino acid residues at positions 11, 37, 44, 45, 47, 83, 84, 103, 104 and 108 according to the Kabat numbering are chosen from the Hallmark residues mentioned in Table 2.

Hence, the (i) at least one ISVD comprised in the polypeptide of the present technology essentially consists of 4 framework regions (FR1 to FR4, respectively) and three complementarity determining regions (CDR1 to CDR3, respectively), wherein the CDRs are as defined herein.

In one embodiment, the ISVD comprised in the polypeptides of the present technology derives from an ISVD, such as from a heavy-chain ISVD, preferably from a Nanobody® ISVD, which has been further engineered/modified to include mutations which prevent/remove binding of pre-existing antibodies/factors. Examples of such mutations are described, e.g., in WO 2012/175741 and WO 2015/173325. For instance, to prevent/remove binding of pre-existing antibodies/factors, the amino acid at position 11 (according to Kabat) may be Val or Leu, preferably Val; and/or the amino acid at position 89 (according to Kabat) may be preferably Val, Thr or Leu, preferably Leu; and/or the amino acid at position 110 (according to Kabat) may be preferably Thr, Lys or Gin, preferably Thr; and/or the amino acid at position 112 (according to Kabat) may be Ser, Lys or Gln, preferably Ser; and/or the ISVD-based building block may contain a C-terminal extension of 1-5 amino acids chosen from any naturally occurring amino acid.

In a preferred embodiment, the polypeptides of the present technology comprise:
(i) at least one immunoglobulin single variable domain (ISVD) which specifically binds to a serum albumin protein; and
(ii) at least one further moiety specifically binding to a serum albumin protein or at least one human serum albumin protein.

The at least one ISVD (i) comprised in the polypeptide of the present technology preferably comprises four framework regions (FR1 to FR4 respectively) and three complementarity determining regions (CDR1 to CDR3, respectively).

The CDR regions are preferably the following (numbering according to AbM):
a) CDR1 comprises the amino acid sequence of SEQ ID NO: 1 or has 3, 2 or 1 amino acid difference(s) with SEQ ID NO: 1 or CDR1 comprises the amino acid sequence of SEQ ID NO: 22 or has 3, 2 or 1 amino acid difference(s) with SEQ ID NO: 22;
b) CDR2 comprises the amino acid sequence of SEQ ID NO: 2 or has 3, 2 or 1 amino acid difference(s) with SEQ ID NO: 2; and
c) CDR3 comprises the amino acid sequence of SEQ ID NO: 3 or has 3, 2 or 1 amino acid difference(s) with SEQ ID NO: 3,
and/or
a) CDR1 comprises the amino acid sequence of SEQ ID NO: 36 or has 3, 2 or 1 amino acid difference(s) with SEQ ID NO: 36;
b) CDR2 comprises the amino acid sequence of SEQ ID NO: 37 or has 3, 2 or 1 amino acid difference(s) with SEQ ID NO: 37; and
c) CDR3 comprises the amino acid sequence of SEQ ID NO: 38 or has 3, 2 or 1 amino acid difference(s) with SEQ ID NO: 38,
and/or
a) CDR1 comprises the amino acid sequence of SEQ ID NO: 55 or has 3, 2 or 1 amino acid difference(s) with SEQ ID NO: 55;
b) CDR2 comprises the amino acid sequence of SEQ ID NO: 56 or has 3, 2 or 1 amino acid difference(s) with SEQ ID NO: 56; and
c) CDR3 comprises the amino acid sequence of SEQ ID NO: 57 or has 3, 2 or 1 amino acid difference(s) with SEQ ID NO: 57.

The above CDR sequences are determined according to AbM numbering.

Preferably, the polypeptide of the present invention comprises, as the first component (i), an albumin binding ISVD. Hence, in a preferred embodiment, the polypeptide of the present invention comprises an albumin-binding ISVD, preferably an albumin-binding ISVD as described herein.

In some embodiments, the at least one ISVD (i) comprised in the polypeptide of the present technology comprises a CDR1 comprising the amino acid sequence of SEQ ID NO: 1, a CDR2 comprising the amino acid sequence of SEQ ID NO: 2; and a CDR3 comprising the amino acid sequence of SEQ ID NO: 3, wherein the CDR sequences are determined according to AbM numbering.

In other embodiments, the at least one ISVD (i) comprised in the polypeptide of the present technology comprises a CDR1 comprising the amino acid sequence of SEQ ID NO: 22, a CDR2 comprising the amino acid sequence of SEQ ID NO: 2; and a CDR3 comprising the amino acid sequence of SEQ ID NO: 3, wherein the CDR sequences are determined according to AbM numbering.

In other embodiments, the at least one ISVD (i) comprised in the polypeptide of the present technology comprises a CDR1 comprising the amino acid sequence of SEQ ID NO: 36, a CDR2 comprising the amino acid sequence of SEQ ID NO: 37; and a CDR3 comprising the amino acid sequence of SEQ ID NO: 38, wherein the CDR sequences are determined according to AbM numbering.

In other embodiments, the at least one ISVD (i) comprised in the polypeptide of the present technology comprises a CDR1 comprising the amino acid sequence of SEQ ID NO: 55, a CDR2 comprising the amino acid sequence of SEQ ID NO: 56; and a CDR3 comprising the amino acid sequence of SEQ ID NO: 57, wherein the CDR sequences are determined according to AbM numbering.

If the CRD sequences are determined according to Kabat numbering, the at least one ISVD (i) comprised in the polypeptide of the present technology would preferably comprise the following CDR regions:
a) CDR1 comprises the amino acid sequence of SEQ ID NO: 27 or has 3, 2 or 1 amino acid difference(s) with SEQ ID NO: 27,
b) CDR2 comprises the amino acid sequence of SEQ ID NO: 28 or has 3, 2 or 1 amino acid difference(s) with SEQ ID NO: 28; and
c) CDR3 comprises the amino acid sequence of SEQ ID NO: 3 or has 3, 2 or 1 amino acid difference(s) with SEQ ID NO: 3,
and/or
a) CDR1 comprises the amino acid sequence of SEQ ID NO: 34 or has 3, 2 or 1 amino acid difference(s) with SEQ ID NO: 34;
b) CDR2 comprises the amino acid sequence of SEQ ID NO: 35 or has 3, 2 or 1 amino acid difference(s) with SEQ ID NO: 35; and
c) CDR3 comprises the amino acid sequence of SEQ ID NO: 38 or has 3, 2 or 1 amino acid difference(s) with SEQ ID NO: 38,
and/or
a) CDR1 comprises the amino acid sequence of SEQ ID NO: 58 or has 3, 2 or 1 amino acid difference(s) with SEQ ID NO: 58;
b) CDR2 comprises the amino acid sequence of SEQ ID NO: 59 or has 3, 2 or 1 amino acid difference(s) with SEQ ID NO: 59; and
c) CDR3 comprises the amino acid sequence of SEQ ID NO: 57 or has 3, 2 or 1 amino acid difference(s) with SEQ ID NO: 57.

In some embodiments, the at least one ISVD (i) comprised in the polypeptide of the present technology comprises a CDR1 comprising the amino acid sequence of SEQ ID NO: 27, a CDR2 comprising the amino acid sequence of SEQ ID NO: 28; and a CDR3 comprising the amino acid sequence of SEQ ID NO: 3, wherein the CDR sequences are determined according to Kabat numbering.

In other embodiments, the at least one ISVD (i) comprised in the polypeptide of the present technology comprises a CDR1 comprising the amino acid sequence of SEQ ID NO: 34, a CDR2 comprising the amino acid sequence of SEQ ID NO: 35; and a CDR3 comprising the amino acid sequence of SEQ ID NO: 38, wherein the CDR sequences are determined according to Kabat numbering.

In other embodiments, the at least one ISVD (i) comprised in the polypeptide of the present technology comprises a CDR1 comprising the amino acid sequence of SEQ ID NO: 58, a CDR2 comprising the amino acid sequence of SEQ ID NO: 59; and a CDR3 comprising the amino acid sequence of SEQ ID NO: 57, wherein the CDR sequences are determined according to Kabat numbering.

Specific examples of ISVDs specifically binding to HSA are ISVDs that comprises 4 framework regions (FR1 to FR4, respectively) and three complementarity determining regions (CDR1 to CDR3), wherein the at least one ISVD specifically binding to a serum albumin protein has:
a) a degree of sequence identity with any one of the sequences as defined in SEQ ID NO's: 4 to 21, 54, or 91 to 93 (in which any C-terminal extension that may be present as well as the CDRs are not taken into account for determining the degree of sequence identity) of at least 85%, preferably at least 90%, more preferably at least 95%; and/or
b) no more than 7, preferably no more than 5, such as only 3, 2 or 1 amino acid differences with any one of the sequences as defined in SEQ ID NO's: 4 to 21, 54, or 91 to 93.

The international application WO 2006/122787, the content of which is herein incorporated by reference, describes a number of ISVDs against (human) serum albumin. These ISVDs include the ISVDs called Alb-1 (SEQ ID NO: 52 in WO 2006/122787) and humanized variants thereof, such as Alb-8 (SEQ ID NO: 62 in WO 2006/122787). Again, these can be used to extend the half-life of biologics according to the present technology.

WO 2012/175400, the content of which is herein incorporated by reference, describes a further improved version of Alb-1, called Alb-23.

In one embodiment, the polypeptide of the present technology comprises a serum albumin binding ISVD selected from Alb-1, Alb-3, Alb-4, Alb-5, Alb-6, Alb-7, Alb-8, Alb-9, Alb-10 (described in WO 2006/122787) and Alb-23. In one embodiment, the serum albumin binding moiety is Alb-8 or Alb-23 or its variants, as shown on pages 7-9 of WO 2012/175400. In one embodiment, the serum albumin binding moiety is selected from the albumin binders described in WO 2012/175741, WO 2015/173325, WO 2017/080850, WO 2017/085172, WO 2018/104444, WO 2018/134235, and WO 2018/134234, the content of which is herein incorporated by reference. Some preferred serum albumin binders are also shown in Table 3A.

TABLE 3A
Serum albumin binding ISVD sequences/HSA sequences
(“ID” refers to the SEQ ID NO as used herein)

| Name | ID | Amino acid sequence |
|---|---|---|
| Alb8 | 5 | EVQLVESGGGLVQPGNSLRLSCAASGFTFSSFGMSWVRQAPGK |
| | | GLEWVSSISGSGSDTLYADSVKGRFTISRDNAKTTLYLQMNSL |
| | | RPEDTAVYYCTIGGSLSRSSQGTLVTVSS |
| Alb23 | 6 | EVQLLESGGGLVQPGGSLRLSCAASGFTFRSFGMSWVRQAPGK |
| | | GPEWVSSISGSGSDTLYADSVKGRFTISRDNSKNTLYLQMNSL |
| | | RPEDTAVYYCTIGGSLSRSSQGTLVTVSS |
| Alb129 | 7 | EVQLVESGGGVVQPGNSLRLSCAASGFTFSSFGMSWVRQAPGK |
| | | GLEWVSSISGSGSDTLYADSVKGRFTISRDNAKTTLYLQMNSL |
| | | RPEDTATYYCTIGGSLSRSSQGTLVTVSSA |
| Alb132 | 8 | EVQLVESGGGVVQPGGSLRLSCAASGFTFRSFGMSWVRQAPGK |
| | | GPEWVSSISGSGSDTLYADSVKGRFTISRDNSKNTLYLQMNSL |
| | | RPEDTATYYCTIGGSLSRSSQGTLVTVSSA |
| Alb11 | 9 | EVQLVESGGGLVQPGNSLRLSCAASGFTFSSFGMSWVRQAPGK |
| | | GLEWVSSISGSGSDTLYADSVKGRFTISRDNAKTTLYLQMNSL |
| | | RPEDTAVYYCTIGGSLSRSSQGTLVTVSS |
| Alb11 | 10 | EVQLVESGGGLVQPGNSLRLSCAASGFTFSSFGMSWVRQAPGK |
| (S112K)-A | | GLEWVSSISGSGSDTLYADSVKGRFTISRDNAKTTLYLQMNSL |
| | | RPEDTAVYYCTIGGSLSRSSQGTLVKVSSA |
| Alb82 | 11 | EVQLVESGGGVVQPGNSLRLSCAASGFTFSSFGMSWVRQAPGK |
| | | GLEWVSSISGSGSDTLYADSVKGRFTISRDNAKTTLYLQMNSL |
| | | RPEDTALYYCTIGGSLSRSSQGTLVTVSS |
| Alb82-A | 12 | EVQLVESGGGVVQPGNSLRLSCAASGFTFSSFGMSWVRQAPGK |
| | | GLEWVSSISGSGSDTLYADSVKGRFTISRDNAKTTLYLQMNSL |
| | | RPEDTALYYCTIGGSLSRSSQGTLVTVSSA |
| Alb82-AA | 13 | EVQLVESGGGVVQPGNSLRLSCAASGFTFSSFGMSWVRQAPGK |
| | | GLEWVSSISGSGSDTLYADSVKGRFTISRDNAKTTLYLQMNSL |
| | | RPEDTALYYCTIGGSLSRSSQGTLVTVSSAA |
| Alb82-AAA | 14 | EVQLVESGGGVVQPGNSLRLSCAASGFTFSSFGMSWVRQAPGK |
| | | GLEWVSSISGSGSDTLYADSVKGRFTISRDNAKTTLYLQMNSL |
| | | RPEDTALYYCTIGGSLSRSSQGTLVTVSSAAA |
| Alb82-G | 15 | EVQLVESGGGVVQPGNSLRLSCAASGFTFSSFGMSWVRQAPGK |
| | | GLEWVSSISGSGSDTLYADSVKGRFTISRDNAKTTLYLQMNSL |
| | | RPEDTALYYCTIGGSLSRSSQGTLVTVSSG |
| Alb82-GG | 16 | EVQLVESGGGVVQPGNSLRLSCAASGFTFSSFGMSWVRQAPGK |
| | | GLEWVSSISGSGSDTLYADSVKGRFTISRDNAKTTLYLQMNSL |
| | | RPEDTALYYCTIGGSLSRSSQGTLVTVSSGG |
| Alb82-GGG | 17 | EVQLVESGGGVVQPGNSLRLSCAASGFTFSSFGMSWVRQAPGK |
| | | GLEWVSSISGSGSDTLYADSVKGRFTISRDNAKTTLYLQMNSL |
| | | RPEDTALYYCTIGGSLSRSSQGTLVTVSSGGG |
| Alb23002 | 4 | EVQLVESGGGVVQPGGSLRLSCAASGFTFRSFGMSWVRQAPGK |
| | | GPEWVSSISGSGSDTLYADSVKGRFTISRDNSKNTLYLQMNSL |
| | | RPEDTALYYCTIGGSLSRSSQGTLVTVSS |
| Alb223 (also | 18 | EVQLVESGGGVVQPGGSLRLSCAASGFTFRSFGMSWVRQAPGK |
| referred to as | | GPEWVSSISGSGSDTLYADSVKGRFTISRDNSKNTLYLQMNSL |
| Alb23002-A) | | RPEDTALYYCTIGGSLSRSSQGTLVTVSSA |
| Alb23002(E1D) | 19 | DVQLVESGGGVVQPGGSLRLSCAASGFTFRSFGMSWVRQAPGK |
| | | GPEWVSSISGSGSDTLYADSVKGRFTISRDNSKNTLYLQMNSL |
| | | RPEDTALYYCTIGGSLSRSSQGTLVTVSS |
| T023500029 | 20 | EVQLVESGGGVVQPGDSLRLSCAASGGTFSTYVMGWFRQAPGK |
| | | EREFVSAISQNSIHTYYANSVKGRFTISRDNSKNTVYLQLNSL |
| | | RPEDTALYYCAASRFTSWYTADYEYDYWGQGTLVTVSS |
| AlbX00001 | 21 | EVQLVESGGGVVQPGGSLRLSCAASGLTFSSYAMGWFRQAPGK |
| | | ERERVVSISRGGGYTYYADSVKGRFTISRDNSENTVYLQMNSL |
| | | RPEDTALYYCAAARYWATGSEYEFDYWGQGTLVTVSS |
| ALBX00002 | 54 | EVQLVESGGGVVQPGGSLRLSCAASGLTFSSYAMGWFRQAPGK |
| | | ERERVVSISRGGGYTYYADSVKGRFTISRDNSKNTVYLQMNSL |
| | | RPEDTALYYCAAARYWATGSEYEFDYWGQGTLVTVSS |
| HSA006A06 | 91 | EVQLVESGGGLVQPGNSLRLSCAASGFTFRSFGMSWVRQAPGK |
| | | EPEWVSSISGSGSDTLYADSVKGRFTISRDNAKTTLYLQMNSL |
| | | KPEDTAVYYCTIGGSLSRSSQGTQVTVSS |
| HSA006A06-A | 92 | EVQLVESGGGLVQPGNSLRLSCAASGFTFRSFGMSWVRQAPGK |
| | | EPEWVSSISGSGSDTLYADSVKGRFTISRDNAKTTLYLQMNSL |
| | | KPEDTAVYYCTIGGSLSRSSQGTQVTVSSA |
| ALB-1 | 93 | AVQLVESGGGLVQPGNSLRLSCAASGFTFRSFGMSWVRQAPGK |
| | | EPEWVSSISGSGSDTLYADSVKGRFTISRDNAKTTLYLQMNSL |
| | | KPEDTAVYYCTIGGSLSRSSQGTQVTVSS |
| AAA98797 | 39 | DAHKSEVAHRFKDLGEENFKALVLIAFAQYLQQCPFEDHVKLVN |
| Human serum | | EVTEFAKTCVADESAENCDKSLHTLFGDKLCTVATLRETYGEMA |
| albumin (1) | | DCCAKQEPERNECFLQHKDDNPNLPRLVRPEVDVMCTAFHDNEE |
| | | TFLKKYLYEIARRHPYFYAPELLFFAKRYKAAFTECCQAADKAA |
| | | CLLPKLDELRDEGKASSAKQRLKCASLQKFGERAFKAWAVARLS |
| | | QRFPKAEFAEVSKLVTDLTKVHTECCHGDLLECADDRADLAKYI |
| | | CENQDSISSKLKECCEKPLLEKSHCIAEVENDEMPADLPSLAAD |
| | | FVESKDVCKNYAEAKDVFLGMFLYEYARRHPDYSVVLLLRLAKT |
| | | YETTLEKCCAAADPHECYAKVFDEFKPLVEEPQNLIKQNCELFK |
| | | QLGEYKFQNALLVRYTKKVPQVSTPTLVEVSRNLGKVGSKCCKH |
| | | PEAKRMPCAEDYLSVVLNQLCVLHEKTPVSDRVTKCCTESLVNR |
| | | RPCFSALEVDETYVPKEFNAETFTFHADICTLSEKERQIKKQTA |
| | | LVELVKHKPKATKEQLKAVMDDFAAFVEKCCKADDKETCFAEEG |
| | | KKLVAASQAALGL |
| P02768-1 Human | 40 | DAHKSEVAHRFKDLGEENFKALVLIAFAQYLQQCPFEDHVKLVN |
| serum albumin | | EVTEFAKTCVADESAENCDKSLHTLFGDKLCTVATLRETYGEMA |
| (2) | | DCCAKQEPERNECFLQHKDDNPNLPRLVRPEVDVMCTAFHDNEE |
| | | TFLKKYLYEIARRHPYFYAPELLFFAKRYKAAFTECCQAADKAA |
| | | CLLPKLDELRDEGKASSAKQRLKCASLQKFGERAFKAWAVARLS |
| | | QRFPKAEFAEVSKLVTDLTKVHTECCHGDLLECADDRADLAKYI |
| | | CENQDSISSKLKECCEKPLLEKSHCIAEVENDEMPADLPSLAAD |
| | | FVESKDVCKNYAEAKDVFLGMFLYEYARRHPDYSVVLLLRLAKT |
| | | YETTLEKCCAAADPHECYAKVFDEFKPLVEEPQNLIKQNCELFE |
| | | QLGEYKFQNALLVRYTKKVPQVSTPTLVEVSRNLGKVGSKCCKH |
| | | PEAKRMPCAEDYLSVVLNQLCVLHEKTPVSDRVTKCCTESLVNR |
| | | RPCFSALEVDETYVPKEFNAETFTFHADICTLSEKERQIKKQTA |
| | | LVELVKHKPKATKEQLKAVMDDFAAFVEKCCKADDKETCFAEEG |
| | | KKLVAASQAALGL |

In some embodiments, the at least one ISVD specifically binding to a serum albumin protein comprised in the polypeptide of the present technology has a sequence that is chosen from the group consisting of SEQ ID NO's: 4 to 21, 54, or 91 to 93 (Table 3A).

In some embodiments, the amino acid sequence of the at least one ISVD specifically binding to a serum albumin protein has a sequence identity of more than 90%, such as more than 95%, or more than 99% with Alb-23002 (SEQ ID NO: 4), with Alb223 (SEQ ID NO.: 18), with Alb23002(E1D) (SEQ ID NO: 19), with ALBX00002 (SEQ ID NO: 54), with Alb82 (SEQ ID NO: 11), with T023500029 (SEQ ID NO: 20), with HSA006A06 (SEQ ID NO: 91), or with HSA006A06-A (SEQ ID NO.: 92).

In one preferred embodiment, the at least one ISVDs specifically binding to a serum albumin protein comprises or consists of the amino acid sequence of Alb-23002 (SEQ ID NO.: 4), or a sequence which has 5, 4, 3, 2 or 1 amino acid difference(s) with SEQ ID NO.: 4. In another preferred embodiment, the at least one ISVDs specifically binding to a serum albumin protein comprises or consists of the amino acid sequence of Alb23002(E1D) (SEQ ID NO.: 19), or a sequence which has 5, 4, 3, 2 or 1 amino acid difference(s) with SEQ ID NO.: 19. In another preferred embodiment, the at least one ISVDs specifically binding to a serum albumin protein comprises or consists of the amino acid sequence of ALBX00002 (SEQ ID NO.: 54), or a sequence which has 5, 4, 3, 2 or 1 amino acid difference(s) with SEQ ID NO.: 54. In another preferred embodiment, the at least one ISVDs specifically binding to a serum albumin protein comprises or consists of the amino acid sequence of T023500029 (SEQ ID NO.:20), or a sequence which has 5, 4, 3, 2 or 1 amino acid difference(s) with SEQ ID NO.: 20. In another preferred embodiment, the at least one ISVDs specifically binding to a serum albumin protein comprises or consists of the amino acid sequence of Alb 82 (SEQ ID NO.:11), or at least 99% identity with the amino acid sequence of SEQ ID NO.: 11, or a sequence which has 5, 4, 3, 2 or 1 amino acid difference(s) with SEQ ID NO.: 11. In another preferred embodiment, the at least one ISVDs specifically binding to a serum albumin protein comprises or consists of the amino acid sequence of Alb223 (SEQ ID NO.:18), or a sequence which has 5, 4, 3, 2 or 1 amino acid difference(s) with SEQ ID NO.: 18. In another preferred embodiment, the at least one ISVDs specifically binding to a serum albumin protein comprises or consists of the amino acid sequence of HSA006A06 (SEQ ID NO.: 91), or a sequence which has 5, 4, 3, 2 or 1 amino acid difference(s) with SEQ ID NO.: 91. In another preferred embodiment, the at least one ISVDs specifically binding to a serum albumin protein comprises or consists of the amino acid sequence of HSA006A06-A (SEQ ID NO.: 92), or a sequence which has 5, 4, 3, 2 or 1 amino acid difference(s) with SEQ ID NO.: 92. In another embodiment, the at least one ISVDs specifically binding to a serum albumin protein comprises or consists of the amino acid sequence of ALB1 (SEQ ID NO.: 93), or a sequence which has 5, 4, 3, 2 or 1 amino acid difference(s) with SEQ ID NO.: 93.

In other embodiments, the amino acid sequence of the at least one ISVD specifically binding to a serum albumin protein has a sequence identity of more than 90%, such as more than 95%, or more than 99% with T023500029 (SEQ ID NO: 20). In other embodiments, the amino acid sequence of the at least one ISVD specifically binding to a serum albumin protein has a sequence identity of more than 90%, such as more than 95%, or more than 99% with Alb-23002 (SEQ ID NO.: 4). In other embodiments, the amino acid sequence of the at least one ISVD specifically binding to a serum albumin protein has a sequence identity of more than 90%, such as more than 95%, or more than 99% with Alb23002(E1D) (SEQ ID NO.: 19). In other embodiments, the amino acid sequence of the at least one ISVD specifically binding to a serum albumin protein has a sequence identity of more than 90%, such as more than 95%, or more than 99% with ALBX00002 (SEQ ID NO.: 54). In other embodiments, the amino acid sequence of the at least one ISVD specifically binding to a serum albumin protein has a sequence identity of more than 90%, such as more than 95%, or more than 99% with Alb 82 (SEQ ID NO.:11). In other embodiments, the amino acid sequence of the at least one ISVD specifically binding to a serum albumin protein has a sequence identity of more than 90%, such as more than 95%, or more than 99% with Alb223 (SEQ ID NO.:18). In other embodiments, the amino acid sequence of the at least one ISVD specifically binding to a serum albumin protein has a sequence identity of more than 90%, such as more than 95%, or more than 99% with HSA006A06 (SEQ ID NO.: 91). In other embodiments, the amino acid sequence of the at least one ISVD specifically binding to a serum albumin protein has a sequence identity of more than 90%, such as more than 95%, or more than 99% with HSA006A06-A (SEQ ID NO.: 92). In other embodiments, the amino acid sequence of the at least one ISVD specifically binding to a serum albumin protein has a sequence identity of more than 90%, such as more than 95%, or more than 99% with ALB1 (SEQ ID NO.: 93).

When an ISVD binding to human serum albumin has 2 or 1 amino acid difference in at least one CDR relative to a corresponding reference CDR sequence (as defined above as SEQ ID NO: 1 to 3, according to AbM numbering) the ISVD preferably has at least half the binding affinity, and more preferably at least the same binding affinity, to human serum albumin as construct ALB23002 (SEQ ID NO: 4), wherein the binding affinity is measured using the same method, such as surface plasmon resonance. When the amino acid sequence of an ISVD binding to human serum albumin has 5, 4, 3, 2 or 1 amino acid difference(s) relative to the amino acid sequence of the corresponding ISVD, the ISVD preferably has at least half the binding affinity, and more preferably at least the same binding affinity, to human serum albumin as construct ALB23002 (SEQ ID NO: 4), wherein the binding affinity is measured using the same method, such as surface plasmon resonance.

In an even more preferred embodiment, the at least one ISVDs specifically binding to a serum albumin protein comprised in the polypeptide of the present technology comprises or consists of the amino acid sequence of Alb-23002 (SEQ ID NO.: 4).

When such an ISVD binding to human serum albumin has a C-terminal position in the polypeptide of the present technology, it may exhibit a C-terminal alanine (A) or C-terminal glycine (G) extension, and is preferably selected from SEQ ID NOs: 7, 8, 10, 12, 13, 14, 15, 16, 17, 92 and 18 (see Table 3A). In a preferred embodiment, the ISVD binding to human serum albumin has another position than the C-terminal position (i.e., is not the C-terminal ISVD of the polypeptide of the technology) and is selected from SEQ ID NOs: 15 to 17 (see Table 3A).

In particular embodiments, the polypeptides as described herein comprising the ISVD as defined herein with the one or more CDRs with 1, 2, 3, or 4 amino acid(s) differences, bind to serum albumin with about the same affinity compared to the binding by the amino acid sequence or polypeptide comprising the CDRs as defined herein without the 4, 3, 2, or 1 amino acid(s) difference, said affinity as measured by surface plasmon resonance.

Compared to the sequence of SEQ ID NO: 4, the at least one serum albumin binding ISVD comprised in the polypeptides of the technology preferably also contain (at least): one or more humanizing substitutions;
and/or
one or more mutations (i.e., amino acid substitutions, deletions or additions, and in particular substitutions) that reduce the binding by pre-existing antibodies; and may optionally contain one or more further mutations (e.g., to improve chemical stability of the polypeptide) as described herein.

For suitable humanizing substitutions (and suitable combinations thereof), reference is for example made to WO 09/138519 (or in the prior art cited in WO 09/138519) and WO 08/020079 (or in the prior art cited in WO 08/020079), as well as Tables A-3 to A-8 from WO 08/020079 (which are lists showing possible humanizing substitutions). Some preferred but non-limiting examples of such humanizing substitutions are Q108L and A14P or a suitable combination thereof. Such humanizing substitutions may also be suitably combined with one or more other mutations as described herein (such as with one or more mutations that reduce binding by pre-existing antibodies).

For suitable mutations that can reduce the binding by pre-existing antibodies (and suitable combinations of such mutations), reference is for example made to WO 2012/175741 and WO 2015/173325 and also to for example WO 2013/024059 and WO 2016/118733.

Generally, if amino acids are substituted in one or more or all of the CDRs, it is preferred that the then-obtained “substituted” sequence is at least 60%, more preferably 65%, even more preferably 70%, particularly preferably 75%, more particularly preferably 80% or even more than 90% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the amino acid sequences, ISVDs or polypeptides may have different degrees of identity to their substituted sequences, e.g., CDR1 may have 80%, while CDR3 may have 90%.

Preferred amino acid substitutions are conservative substitutions. Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gin; (c) polar, positively charged residues: His, Arg and Lys; (d) large aliphatic, nonpolar residues: Met, Leu, lie, Val and Cys; and (e) aromatic residues: Phe, Tyr and Trp. Further preferred conservative substitutions are as follows: Ala into Gly or into Ser; Arg into Lys; Asn into Gin or into His; Asp nto Glu; Cys into Ser; Gln into Asn; Glu into Asp; Gly into Ala or into Pro; His into Asn or into GIn; lie into Leu or into Val; Leu into lie or into Val; Lys into Arg, into Gin or into Glu; Met into Leu, into Tyr or into lie; Phe into Met, into Leu or into Tyr; Ser into Thr; Thr into Ser; Trp into Tyr; Tyr into Trp; and/or Phe into Val, into Ile or into Leu. However, any substitution (including non-conservative substitution) is envisaged as long as the polypeptide retains its capability to specifically bind to the epitope on albumin as described herein, and/or its CDRs have an identity of at least 60%, more preferably 65%, even more preferably 70%, particularly preferably 75%, more particularly preferably 80% to the “original” CDR sequence.

In some embodiments, the ISVD is a (single) domain antibody, a V_{HH}, a humanized V_{HH}, or a camelized V_{H}, preferably a Nanobody®.

Preferably, the first domain (i) comprised in the polypeptide of the present technology is or comprises at least one ISVD which specifically binds to a serum albumin protein, as described herein.

In another embodiment, the first domain (i) comprised in the polypeptide of the present technology may be or comprise at least one human serum albumin protein. The serum albumin protein may be human serum albumin (HSA, such as, e.g., AAA98797 as defined in SEQ ID NO: 39 or P02768-1 as defined in SEQ ID NO: 40) or derivatives, variants, or fragments thereof, as described in detail below. In one embodiment, the first domain (i) comprised in the polypeptide of the present technology is or comprises at least one HSA protein as defined in SEQ ID NO.: 40, or a HSA protein with an amino acid sequence which has at least 85%, such as at least 90%, or at least 95%, or at least 99% identity with the amino acid sequence of SEQ ID NO.: 40.

### Second Domain: At Least One Further Moiety Specifically Binding to a Serum Albumin Protein or at Least One Human Serum Albumin Protein

In particular embodiments, the polypeptides as disclosed herein comprise (i) at least one ISVD, as defined herein, and at (ii) least one moiety comprising a serum albumin protein.

In particular embodiments, the serum albumin protein is human serum albumin (AAA98797 as defined in SEQ ID NO: 39 or P02768-1 as defined in SEQ ID NO: 40) or a polymorphic variant or isoform thereof. Preferably, the serum albumin protein is human serum albumin is or comprises a protein sequence as defined in SEQ ID NO.: 40, or a HSA with an amino acid sequence which has at least 85%, such as at least 90%, or at least 95%, or at least 99% identity with the amino acid sequence of SEQ ID NO.: 40.

In the context of the present technology, the term “serum albumin protein” means serum albumin, such as human serum albumin (e.g., as defined in SEQ ID NO's: 39 and 40), or derivatives, variants, or fragments thereof.

The size of the albumin derivative, variant, or fragment thereof may vary depending on the size of the fragment, number of domains, the size of the non-albumin part of the polypeptide etc. It is preferred however that the albumin derivative, variant, or fragment has a size in the range of 40-80 kDa, preferably in the range of 50-70 kDa, more preferred in the range of 55-65 kDa and most preferred around 60 kDa.

Human serum albumin is the preferred serum albumin protein according to the technology and is a protein consisting of about 585 amino acid residues and has a molecular weight of about 67 kDa (e.g., SEQ ID NO: 39 or SEQ ID NO: 40, preferably SEQ ID NO.: 40). The skilled person will appreciate that natural alleles may exist having essentially the same properties as human serum albumin but having one or more (several) amino acid changes compared to SEQ ID NO: 39 or SEQ ID NO: 40, and the inventors also contemplate the use of such natural alleles as serum albumin proteins according to the technology.

According to the technology the term “(serum) albumin derivative” means a non-natural, engineered molecule comprising or consisting of one or more parts of one or more domains of a serum albumin protein as specified.

The term “(serum) albumin variant” includes an albumin or albumin derivative in which the albumin or albumin derivative is altered by chemical means such as post-translational derivatization or modification of the polypeptide, e.g., PEGylation and/or conjugation of a desirable moiety (such as a therapeutic moiety) to a thiol group, such as provided by an unpaired cysteine. The terms “derivative” and “variant” may or may not be used interchangeably.

The term “(serum) albumin fragment” means a polypeptide having one or more (several) amino acids deleted from the amino and/or carboxyl terminus of a serum albumin protein and/or an internal region of a serum albumin protein that has retained the ability to bind to FcRn. Fragments may comprise or consist of one uninterrupted sequence derived from human serum albumin or it may comprise or consist of two or more sequences derived from human serum albumin.

In particular embodiments, the at least one further moiety that comprises a serum albumin protein is a part, a fragment, a derivative or variant of a serum albumin protein.

In particular embodiments, the at least one moiety that comprises a serum albumin protein is human serum albumin or a part, a fragment, a derivative or variant of human serum albumin.

In other particular embodiments, the polypeptides as disclosed herein comprise (ii) at least one further moiety specifically binding to a serum albumin protein, such as specifically binding to human serum albumin (AAA98797 as defined in SEQ ID NO: 39 or P02768-1 as defined in SEQ ID NO: 40) or (polymorphic) variants or isoforms thereof.

Preferably, the polypeptide of the present technology comprises as the second component (ii) at least one further moiety specifically binding to a serum albumin protein, such as specifically binding to human serum albumin (AAA98797 as defined in SEQ ID NO: 39 or P02768-1 as defined in SEQ ID NO: 40) or (polymorphic) variants or isoforms thereof.

In further particular embodiments, the (ii) at least one further moiety specifically binding to a serum albumin protein comprised in the polypeptides of the present technology specifically binds to amino acid residues on the serum albumin protein that are not involved in binding of the serum albumin protein to FcRn.

In further particular embodiments, the (ii) at least one further moiety specifically binding to a serum albumin protein comprised in the polypeptides of the present technology specifically binds to domain II of human serum albumin.

In particular embodiments, the (ii) at least one further moiety that specifically binds to a serum albumin protein comprised in the polypeptides of the present technology is a peptide or protein comprising between 5 and 500 amino acids.

In yet further particular embodiments, the (ii) at least one moiety specifically binding to a serum albumin protein comprised in the polypeptides of the present technology is chosen from the group consisting of an Affibody®, a scFv, a Fab, a Designed Ankyrin Repeat Protein (DARPin®), an Albumin Binding Domain (ABD), a Nanofitin® (aka affitin) and an immunoglobulin variable domain sequence (ISVD).

Albumin binding domains (ABD) are described, e.g., in Hopp J. et al., “The effects of affinity and valency of an albumin-binding domain (ABD) on the half-life of a single-chain diabody-ABD fusion protein”, Protein Eng Des Sel., 2010, 23(11):827-34. For instance, the polypeptide of the present technology may comprise an ABD which comprises or consist of SEQ ID NO.: 90 (LKEAKEKAIEELKKAGITSDYYFDLINKAKTVEGVNALKDEILKA).

The (ii) at least one further moiety specifically binding to a serum albumin protein may preferably be an albumin-binding ISVD as described in the context of component (i) of the polypeptide of the present technology. For instance, the (ii) at least one further moiety specifically binding to a serum albumin protein may be an ISVD which comprises a CDR1 comprising the amino acid sequence of SEQ ID NO: 1, a CDR2 comprising the amino acid sequence of SEQ ID NO: 2; and a CDR3 comprising the amino acid sequence of SEQ ID NO: 3, wherein the CDR sequences are determined according to AbM numbering. For instance, the (ii) at least one further moiety specifically binding to a serum albumin protein may be an ISVD which comprises a CDR1 comprising the amino acid sequence of SEQ ID NO: 22, a CDR2 comprising the amino acid sequence of SEQ ID NO: 2; and a CDR3 comprising the amino acid sequence of SEQ ID NO: 3, wherein the CDR sequences are determined according to AbM numbering. For instance, the (ii) at least one further moiety specifically binding to a serum albumin protein may be an ISVD which comprises a CDR1 comprising the amino acid sequence of SEQ ID NO: 36, a CDR2 comprising the amino acid sequence of SEQ ID NO: 37; and a CDR3 comprising the amino acid sequence of SEQ ID NO: 38, wherein the CDR sequences are determined according to AbM numbering. For instance, the (ii) at least one further moiety specifically binding to a serum albumin protein may be an ISVD which comprises a CDR1 comprising the amino acid sequence of SEQ ID NO: 55, a CDR2 comprising the amino acid sequence of SEQ ID NO: 56; and a CDR3 comprising the amino acid sequence of SEQ ID NO: 57, wherein the CDR sequences are determined according to AbM numbering. For instance, the (ii) at least one further moiety specifically binding to a serum albumin protein may be an ISVD which comprises or consists of a sequence as defined in any one of SEQ ID NOs: 4 to 21, 54, or 91 to 93, or a sequence which as at least 90%, such as at least 95%, or at least 99% with the amino acid sequence of any one of SEQ ID NOs.: 4 to 21, 54, or 91 to 93.

In further particular embodiments, the (ii) at least one moiety specifically binding to a serum albumin protein comprised in the polypeptide of the present technology is at least one ISVD specifically binding to domain II of serum albumin, such as domain II of human serum albumin.

In other embodiments, the (ii) at least one moiety specifically binding to a serum albumin protein comprised in the polypeptide of the present technology is at least one DARPin specifically binding to serum albumin. For instance, the at least one DARPin may comprise or consist of SEQ ID NO.: 88, or a polypeptide with at least 90%, such as at least 95%, or at least 97%, or at least 99% sequence identity with SEQ ID NO.: 88.

In other embodiments, the (ii) at least one moiety specifically binding to a serum albumin protein comprised in the polypeptide of the present technology is at least one Affitin specifically binding to serum albumin. For instance, the at least one Affitin may comprise or consist of SEQ ID NO.: 89, or a polypeptide with at least 90%, such as at least 95%, or at least 97%, or at least 99% sequence identity with SEQ ID NO.: 89.

In other embodiments, the (ii) at least one moiety specifically binding to a serum albumin protein comprised in the polypeptide of the present technology is at least one albumin-binding domain (ABD) specifically binding to serum albumin. For instance, the at least one ABD may comprise or consist of SEQ ID NO.: 90, or a polypeptide with at least 90%, such as at least 95%, or at least 97%, or at least 99% sequence identity with SEQ ID NO.: 90.

TABLE 3B
Serum albumin binding protein sequences
(“ID” refers to the SEQ ID NO as used
herein)

| Name | ID | Amino acid sequence |
|---|---|---|
| Darpin | 88 | DLGKKLLEAARAGQDDEVRELLKAGADVN |
| | | AKDYFSHTPLHLAARNGHLKIVEVLLKAG |
| | | ADVNAKDFAGKTPLHLAANEGHLEIVEVL |
| | | LKAGADVNAQDIFGKTPADIAADAGHEDI |
| | | AEVLQKAA |
| Affitin | 89 | VKVKFWPRGEEKVVDTSKIAWVLRADKTV |
| (Nanofitin) | | MFKYDDNGKKGYGVVLEKDAPKELLDMLA |
| | | RAEREK |
| ABD | 90 | LKEAKEKAIEELKKAGITSDYYFDLINKA |
| | | KTVEGVNALKDEILKA |

### Polypeptides of the Present Technology

Hence, as described above, the polypeptides of the present technology comprise (i) at least one immunoglobulin single variable domain (ISVD) which specifically binds to a serum albumin protein or at least one human serum albumin protein and (ii) at least one further moiety specifically binding to a serum albumin protein or at least one human serum albumin protein.

Component (i) and component (ii) may be directly linked to each other (as for example described in WO 99/23221) and/or may be linked to each other via one or more suitable spacers or linkers, or any combination thereof.

Suitable spacers or linkers for use in the polypeptides will be clear to the skilled person, and may generally be any linker or spacer used in the art to link amino acid sequences. Preferably, said linker or spacer is suitable for use in constructing proteins or polypeptides that are intended for pharmaceutical use.

For example, a linker may be a suitable amino acid sequence, and in particular amino acid sequences of between 1 and 50, preferably between 1 and 30, such as between 1 and 10 amino acid residues. Some preferred examples of such amino acid sequences include gly-ser linkers, for example of the type (glyₓser_{y})_{z}, such as (for example (gly₄ser)₃ or (gly₃ser₂)₃, as described in WO 99/42077, and the 30GS, 15GS, 9GS and 7GS linkers described in the applications by Ablynx mentioned herein (see, for example WO 06/040153 and WO 06/122825), as well as hinge-like regions, such as the hinge regions of naturally occurring heavy chain antibodies or similar sequences (such as described in WO 94/04678). Preferred linkers are depicted in Table 4, in particular 9GS, 20GS and 35GS, SEQ ID NO.: 45, 49 or 52, more preferably 9GS and 35GS, and most preferably a 35GS linker.

TABLE 4
Linker sequences (“ID” refers to the
SEQ ID NO. as used herein)

| Name | ID | Amino acid sequence |
|---|---|---|
| 3A linker | 41 | AAA |
| 5GS linker | 42 | GGGGS |
| 7GS linker | 43 | SGGSGGS |
| 8GS linker | 44 | GGGGSGGS |
| 9GS linker | 45 | GGGGSGGGS |
| 10GS linker | 46 | GGGGSGGGGS |
| 15GS linker | 47 | GGGGSGGGGSGGGGS |
| 18GS linker | 48 | GGGGSGGGGSGGGGSGGS |
| 20GS linker | 49 | GGGGSGGGGSGGGGSGGGGS |
| 25GS linker | 50 | GGGGSGGGGSGGGGSGGGGSGGGGS |
| 30GS linker | 51 | GGGGSGGGGSGGGGSGGGGSGGGGSGGGGS |
| 35GS linker | 52 | GGGGSGGGGSGGGGSGGGGSGGGGSGGGGS |
| | | GGGGS |
| 40GS linker | 53 | GGGGSGGGGSGGGGSGGGGSGGGGSGGGGS |
| | | GGGGSGGGGS |

Some other particularly preferred linkers are poly-alanine (such as AAA), as well as the linkers GS30 (SEQ ID NO: 85 in WO 06/122825) and GS9 (SEQ ID NO: 84 in WO 06/122825).

The length, the degree of flexibility and/or other properties of the linker(s) used (although not critical, as it usually is for linkers used in ScFv fragments) may have some influence on the properties of the final polypeptide of the technology, including but not limited to the affinity, specificity or avidity for albumin, or for one or more of the other antigens. Based on the disclosure herein, the skilled person will be able to determine the optimal linker(s) for use in a specific polypeptide of the technology, optionally after some limited routine experiments.

Other suitable linkers generally comprise organic compounds or polymers, in particular those suitable for use in proteins for pharmaceutical use. For instance, poly(ethyleneglycol) moieties have been used to link antibody domains, see for example WO 2004/081026.

In one embodiment, the polypeptide of the present technology comprises, as components (i) and (ii), two ISVDs, wherein each ISVD comprises, independently, three complementarity determining regions (CDR1 to CDR3, respectively), wherein:
a) CDR1 comprises the amino acid sequence of SEQ ID NO: 1 or has 3, 2 or 1 amino acid difference(s) with SEQ ID NO: 1 or CDR1 comprises the amino acid sequence of SEQ ID NO: 22 or has 3, 2 or 1 amino acid difference(s) with SEQ ID NO: 22
b) CDR2 comprises the amino acid sequence of SEQ ID NO: 2 or has 3, 2 or 1 amino acid difference(s) with SEQ ID NO: 2; and
c) CDR3 comprises the amino acid sequence of SEQ ID NO: 3 or has 3, 2 or 1 amino acid difference(s) with SEQ ID NO: 3,
   and/or
a) CDR1 comprises the amino acid sequence of SEQ ID NO: 36 or has 3, 2 or 1 amino acid difference(s) with SEQ ID NO: 36;
b) CDR2 comprises the amino acid sequence of SEQ ID NO: 37 or has 3, 2 or 1 amino acid difference(s) with SEQ ID NO: 37; and
c) CDR3 comprises the amino acid sequence of SEQ ID NO: 38 or has 3, 2 or 1 amino acid difference(s) with SEQ ID NO: 38,
   and/or
a) CDR1 comprises the amino acid sequence of SEQ ID NO: 55 or has 3, 2 or 1 amino acid difference(s) with SEQ ID NO: 55;
b) CDR2 comprises the amino acid sequence of SEQ ID NO: 56 or has 3, 2 or 1 amino acid difference(s) with SEQ ID NO: 56; and
c) CDR3 comprises the amino acid sequence of SEQ ID NO: 57 or has 3, 2 or 1 amino acid difference(s) with SEQ ID NO: 57
   wherein the CDR sequences are determined according to AbM numbering.

Hence, in one embodiment, the polypeptide of the present technology comprises:
(i) at least one immunoglobulin single variable domain (ISVD) which specifically binds to a serum albumin protein wherein said (i) at least one ISVD comprises three complementarity determining regions (CDR1 to CDR3, respectively), wherein:
   a) CDR1 comprises the amino acid sequence of SEQ ID NO: 1 or has 3, 2 or 1 amino acid difference(s) with SEQ ID NO: 1 or CDR1 comprises the amino acid sequence of SEQ ID NO: 22 or has 3, 2 or 1 amino acid difference(s) with SEQ ID NO: 22
   b) CDR2 comprises the amino acid sequence of SEQ ID NO: 2 or has 3, 2 or 1 amino acid difference(s) with SEQ ID NO: 2; and
   c) CDR3 comprises the amino acid sequence of SEQ ID NO: 3 or has 3, 2 or 1 amino acid difference(s) with SEQ ID NO: 3,
and/or
   a) CDR1 comprises the amino acid sequence of SEQ ID NO: 36 or has 3, 2 or 1 amino acid difference(s) with SEQ ID NO: 36;
   b) CDR2 comprises the amino acid sequence of SEQ ID NO: 37 or has 3, 2 or 1 amino acid difference(s) with SEQ ID NO: 37; and
   c) CDR3 comprises the amino acid sequence of SEQ ID NO: 38 or has 3, 2 or 1 amino acid difference(s) with SEQ ID NO: 38,
and/or
   a) CDR1 comprises the amino acid sequence of SEQ ID NO: 55 or has 3, 2 or 1 amino acid difference(s) with SEQ ID NO: 55;
   b) CDR2 comprises the amino acid sequence of SEQ ID NO: 56 or has 3, 2 or 1 amino acid difference(s) with SEQ ID NO: 56; and
   c) CDR3 comprises the amino acid sequence of SEQ ID NO: 57 or has 3, 2 or 1 amino acid difference(s) with SEQ ID NO: 57,
      wherein the CDR sequences are determined according to AbM numbering; and
(ii) at least one further moiety specifically binding to a serum albumin protein, wherein the at least one further moiety specifically binding to a serum albumin protein is an ISVD, wherein said (ii) ISVD comprises three complementarity determining regions (CDR1 to CDR3, respectively), wherein:
   a) CDR1 comprises the amino acid sequence of SEQ ID NO: 1 or has 3, 2 or 1 amino acid difference(s) with SEQ ID NO: 1 or CDR1 comprises the amino acid sequence of SEQ ID NO: 22 or has 3, 2 or 1 amino acid difference(s) with SEQ ID NO: 22
   b) CDR2 comprises the amino acid sequence of SEQ ID NO: 2 or has 3, 2 or 1 amino acid difference(s) with SEQ ID NO: 2; and
   c) CDR3 comprises the amino acid sequence of SEQ ID NO: 3 or has 3, 2 or 1 amino acid difference(s) with SEQ ID NO: 3,
and/or
   a) CDR1 comprises the amino acid sequence of SEQ ID NO: 36 or has 3, 2 or 1 amino acid difference(s) with SEQ ID NO: 36;
   b) CDR2 comprises the amino acid sequence of SEQ ID NO: 37 or has 3, 2 or 1 amino acid difference(s) with SEQ ID NO: 37; and
   c) CDR3 comprises the amino acid sequence of SEQ ID NO: 38 or has 3, 2 or 1 amino acid difference(s) with SEQ ID NO: 38,
and/or
   a) CDR1 comprises the amino acid sequence of SEQ ID NO: 55 or has 3, 2 or 1 amino acid difference(s) with SEQ ID NO: 55;
   b) CDR2 comprises the amino acid sequence of SEQ ID NO: 56 or has 3, 2 or 1 amino acid difference(s) with SEQ ID NO: 56; and
   c) CDR3 comprises the amino acid sequence of SEQ ID NO: 57 or has 3, 2 or 1 amino acid difference(s) with SEQ ID NO: 57,
      wherein the CDR sequences are determined according to AbM numbering.

The two ISVDs comprised as components (i) and (ii) may be the same or different, and each of them comprises, preferably, three CDRs as defined above. Hence, in one embodiment, the two ISVDs comprised as components (i) and (ii) in the polypeptide of the present technology are ISVDs which comprise or consist of a sequence selected from SEQ ID NO.: 4 to 21, 54, or 91 to 93, or a sequences which comprises or consists of a sequence with at least 90%, such as at least 95%, or at least 97%, or at least 99% sequence identity with a sequence selected from SEQ ID NO.: 4 to 21, 54, or 91 to 93. Examples of polypeptides comprising two ISVDs as components (i) and (ii) are depicted Table 5 as SEQ ID NO.: 74-78, 147-149 and 152-154. Hence, in one embodiment, the polypeptide of the present technology may comprise two ISVDs, which may be the same or different, linked directly or through a peptide linker, preferably through a peptide linker, such as a peptide linker as depicted in Table 4, e.g., a 9GS linker (SEQ ID NO.: 45) or a 35GS linker (SEQ ID NO.: 52).

For instance, component (i) may be selected from an ISVD comprising or consisting of SEQ ID NO.: 11, 12, 54, 18, 4, 20, 91 or 92, or a protein comprising or consisting of a sequence with at least 90%, such as at least 95%, or at least 97%, or at least 99% sequence identity with SEQ ID NO.: 11, 12, 54, 18, 4, 20, 91 or 92. For instance, component (ii) may be selected from an ISVD comprising or consisting of SEQ ID NO.: 11, 12, 54, 18, 4, 20, 91 or 92, or a protein comprising or consisting of a sequence with at least 90%, such as at least 95%, or at least 97%, or at least 99% sequence identity with SEQ ID NO.: 11, 12, 54, 18, 4, 20, 91 or 92. Component (i) and component (ii) may be linked directly orthrough a peptide linker, preferably through a peptide linker, such as a peptide linker as depicted in Table 4, e.g., a 9GS linker (SEQ ID NO.: 45) or a 35GS linker (SEQ ID NO.: 52).

In one embodiment, the polypeptide of the present technology comprises as component (i) an ISVD comprising or consisting of SEQ ID NO.: 91, or a protein comprising or consisting of a sequence with at least 90%, such as at least 95%, or at least 97%, or at least 99% sequence identity with SEQ ID NO.: 91, and, as component (ii) an ISVDs comprising or consisting of SEQ ID NO.: 92, or a protein comprising or consisting of a sequence with at least 90%, such as at least 95%, or at least 97%, or at least 99% sequence identity with SEQ ID NO.: 92. Components (i) and (ii) may be linked directly or through a peptide linker, preferably through a peptide linker, such as a peptide linker as depicted in Table 4, e.g., a 9GS linker (SEQ ID NO.: 45) or a 35GS linker (SEQ ID NO.: 52). For instance, the polypeptide may comprise or consist of SEQ ID NO 74, or a polypeptide comprising or consisting of a sequence with at least 90%, such as at least 95%, or at least 97%, or at least 99% sequence identity with SEQ ID NO.: 74.

In one embodiment, the polypeptide of the present technology comprises as components (i) and (ii) two ISVDs comprising or consisting of SEQ ID NO.: 91, or a protein comprising or consisting of a sequence with at least 90%, such as at least 95%, or at least 97%, or at least 99% sequence identity with SEQ ID NO.: 91. Components (i) and (ii) may be linked directly or through a peptide linker, preferably through a peptide linker, such as a peptide linker as depicted in Table 4, e.g., a 9GS linker (SEQ ID NO.: 45) or a 35GS linker (SEQ ID NO.: 52).

In one embodiment, the polypeptide of the present technology comprises as component (i) an ISVD comprising or consisting of SEQ ID NO.: 91, or a protein comprising or consisting of a sequence with at least 90%, such as at least 95%, or at least 97%, or at least 99% sequence identity with SEQ ID NO.: 91, and, as component (ii) an ISVDs comprising or consisting of SEQ ID NO.: 18, or a protein comprising or consisting of a sequence with at least 90%, such as at least 95%, or at least 97%, or at least 99% sequence identity with SEQ ID NO.: 18. Components (i) and (ii) may be linked directly or through a peptide linker, preferably through a peptide linker, such as a peptide linker as depicted in Table 4, e.g., a 9GS linker (SEQ ID NO.: 45) or a 35GS linker (SEQ ID NO.: 52). For instance, the polypeptide may comprise or consist of SEQ ID NO 76, or a polypeptide comprising or consisting of a sequence with at least 90%, such as at least 95%, or at least 97%, or at least 99% sequence identity with SEQ ID NO.: 76.

In one embodiment, the polypeptide of the present technology comprises as component (i) an ISVD comprising or consisting of SEQ ID NO.: 91, or a protein comprising or consisting of a sequence with at least 90%, such as at least 95%, or at least 97%, or at least 99% sequence identity with SEQ ID NO.: 90, and, as component (ii) an ISVDs comprising or consisting of SEQ ID NO.: 4, or a protein comprising or consisting of a sequence with at least 90%, such as at least 95%, or at least 97%, or at least 99% sequence identity with SEQ ID NO.: 4. Components (i) and (ii) may be linked directly or through a peptide linker, preferably through a peptide linker, such as a peptide linker as depicted in Table 4, e.g., a 9GS linker (SEQ ID NO.: 45) or a 35GS linker (SEQ ID NO.: 52).

In one embodiment, the polypeptide of the present technology comprises as component (i) an ISVD comprising or consisting of SEQ ID NO.: 11, or a protein comprising or consisting of a sequence with at least 90%, such as at least 95%, or at least 97%, or at least 99% sequence identity with SEQ ID NO.: 11, and, as component (ii) an ISVDs comprising or consisting of SEQ ID NO.: 12, or a protein comprising or consisting of a sequence with at least 90%, such as at least 95%, or at least 97%, or at least 99% sequence identity with SEQ ID NO.: 12. Components (i) and (ii) may be linked directly or through a peptide linker, preferably through a peptide linker, such as a peptide linker as depicted in Table 4, e.g., a 9GS linker (SEQ ID NO.: 45) or a 35GS linker (SEQ ID NO.: 52).

In one embodiment, the polypeptide of the present technology comprises as components (i) and (ii) two ISVDs comprising or consisting of SEQ ID NO.: 11, or a protein comprising or consisting of a sequence with at least 90%, such as at least 95%, or at least 97%, or at least 99% sequence identity with SEQ ID NO.: 11. Components (i) and (ii) may be linked directly or through a peptide linker, preferably through a peptide linker, such as a peptide linker as depicted in Table 4, e.g., a 9GS linker (SEQ ID NO.: 45) or a 35GS linker (SEQ ID NO.: 52).

In one embodiment, the polypeptide of the present technology comprises as component (i) an ISVD comprising or consisting of SEQ ID NO.: 54, or a protein comprising or consisting of a sequence with at least 90%, such as at least 95%, or at least 97%, or at least 99% sequence identity with SEQ ID NO.: 54, and, as component (ii) an ISVDs comprising or consisting of SEQ ID NO.: 18, or a protein comprising or consisting of a sequence with at least 90%, such as at least 95%, or at least 97%, or at least 99% sequence identity with SEQ ID NO.: 18. Components (i) and (ii) may be linked directly or through a peptide linker, preferably through a peptide linker, such as a peptide linker as depicted in Table 4, e.g., a 9GS linker (SEQ ID NO.: 45) or a 35GS linker (SEQ ID NO.: 52).

In one embodiment, the polypeptide of the present technology comprises as component (i) an ISVD comprising or consisting of SEQ ID NO.: 54, or a protein comprising or consisting of a sequence with at least 90%, such as at least 95%, or at least 97%, or at least 99% sequence identity with SEQ ID NO.: 54, and, as component (ii) an ISVDs comprising or consisting of SEQ ID NO.: 4, or a protein comprising or consisting of a sequence with at least 90%, such as at least 95%, or at least 97%, or at least 99% sequence identity with SEQ ID NO.: 4. Components (i) and (ii) may be linked directly or through a peptide linker, preferably through a peptide linker, such as a peptide linker as depicted in Table 4, e.g., a 9GS linker (SEQ ID NO.: 45) or a 35GS linker (SEQ ID NO.: 52).

In one embodiment, the polypeptide of the present technology comprises as component (i) an ISVD comprising or consisting of SEQ ID NO.: 54, or a protein comprising or consisting of a sequence with at least 90%, such as at least 95%, or at least 97%, or at least 99% sequence identity with SEQ ID NO.: 54, and, as component (ii) an ISVDs comprising or consisting of SEQ ID NO.: 18, or a protein comprising or consisting of a sequence with at least 90%, such as at least 95%, or at least 97%, or at least 99% sequence identity with SEQ ID NO.: 18. Components (i) and (ii) may be linked directly or through a peptide linker, preferably through a peptide linker, such as a peptide linker as depicted in Table 4, e.g., a 9GS linker (SEQ ID NO.: 45) or a 35GS linker (SEQ ID NO.: 52). For instance, the polypeptide may comprise or consist of SEQ ID NO 77, or a polypeptide comprising or consisting of a sequence with at least 90%, such as at least 95%, or at least 97%, or at least 99% sequence identity with SEQ ID NO.: 77.

In one embodiment, the polypeptide of the present technology comprises as component (i) an ISVD comprising or consisting of SEQ ID NO.: 20, or a protein comprising or consisting of a sequence with at least 90%, such as at least 95%, or at least 97%, or at least 99% sequence identity with SEQ ID NO.: 20, and, as component (ii) an ISVDs comprising or consisting of SEQ ID NO.: 18, or a protein comprising or consisting of a sequence with at least 90%, such as at least 95%, or at least 97%, or at least 99% sequence identity with SEQ ID NO.: 18. Components (i) and (ii) may be linked directly or through a peptide linker, preferably through a peptide linker, such as a peptide linker as depicted in Table 4, e.g., a 9GS linker (SEQ ID NO.: 45) or a 35GS linker (SEQ ID NO.: 52). For instance, the polypeptide may comprise or consist of SEQ ID NO 78, or a polypeptide comprising or consisting of a sequence with at least 90%, such as at least 95%, or at least 97%, or at least 99% sequence identity with SEQ ID NO.: 78. In one embodiment, the polypeptide of the present technology comprises as component (i) an ISVD comprising or consisting of SEQ ID NO.: 20, or a protein comprising or consisting of a sequence with at least 90%, such as at least 95%, or at least 97%, or at least 99% sequence identity with SEQ ID NO.: 20, and, as component (ii) an ISVDs comprising or consisting of SEQ ID NO.: 4, or a protein comprising or consisting of a sequence with at least 90%, such as at least 95%, or at least 97%, or at least 99% sequence identity with SEQ ID NO.: 4. Components (i) and (ii) may be linked directly or through a peptide linker, preferably through a peptide linker, such as a peptide linker as depicted in Table 4, e.g., a 9GS linker (SEQ ID NO.: 45) or a 35GS linker (SEQ ID NO.: 52).

In one embodiment, the polypeptide of the present technology comprises as component (i) an ISVD comprising or consisting of SEQ ID NO.: 88, or a protein comprising or consisting of a sequence with at least 90%, such as at least 95%, or at least 97%, or at least 99% sequence identity with SEQ ID NO.: 88, and, as component (ii) an ISVDs comprising or consisting of SEQ ID NO.: 18, or a protein comprising or consisting of a sequence with at least 90%, such as at least 95%, or at least 97%, or at least 99% sequence identity with SEQ ID NO.: 18. Components (i) and (ii) may be linked directly or through a peptide linker, preferably through a peptide linker, such as a peptide linker as depicted in Table 4, e.g., a 9GS linker (SEQ ID NO.: 45) or a 35GS linker (SEQ ID NO.: 52). For instance, the polypeptide may comprise or consist of SEQ ID NO 79, or a polypeptide comprising or consisting of a sequence with at least 90%, such as at least 95%, or at least 97%, or at least 99% sequence identity with SEQ ID NO.: 79.

In one embodiment, the polypeptide of the present technology comprises as component (i) an ISVD comprising or consisting of SEQ ID NO.: 88, or a protein comprising or consisting of a sequence with at least 90%, such as at least 95%, or at least 97%, or at least 99% sequence identity with SEQ ID NO.: 88, and, as component (ii) an ISVDs comprising or consisting of SEQ ID NO.: 4, or a protein comprising or consisting of a sequence with at least 90%, such as at least 95%, or at least 97%, or at least 99% sequence identity with SEQ ID NO.: 4. Components (i) and (ii) may be linked directly or through a peptide linker, preferably through a peptide linker, such as a peptide linker as depicted in Table 4, e.g., a 9GS linker (SEQ ID NO.: 45) or a 35GS linker (SEQ ID NO.: 52).

In one embodiment, the polypeptide of the present technology comprises as component (i) an ISVD comprising or consisting of SEQ ID NO.: 90, or a protein comprising or consisting of a sequence with at least 90%, such as at least 95%, or at least 97%, or at least 99% sequence identity with SEQ ID NO.: 90, and, as component (ii) an ISVDs comprising or consisting of SEQ ID NO.: 18, or a protein comprising or consisting of a sequence with at least 90%, such as at least 95%, or at least 97%, or at least 99% sequence identity with SEQ ID NO.: 18. Components (i) and (ii) may be linked directly or through a peptide linker, preferably through a peptide linker, such as a peptide linker as depicted in Table 4, e.g., a 9GS linker (SEQ ID NO.: 45) or a 35GS linker (SEQ ID NO.: 52). For instance, the polypeptide may comprise or consist of SEQ ID NO.: 81, or a polypeptide comprising or consisting of a sequence with at least 90%, such as at least 95%, or at least 97%, or at least 99% sequence identity with SEQ ID NO.: 81.

In one embodiment, the polypeptide of the present technology comprises as component (i) an ISVD comprising or consisting of SEQ ID NO.: 90, or a protein comprising or consisting of a sequence with at least 90%, such as at least 95%, or at least 97%, or at least 99% sequence identity with SEQ ID NO.: 90, and, as component (ii) an ISVDs comprising or consisting of SEQ ID NO.: 4, or a protein comprising or consisting of a sequence with at least 90%, such as at least 95%, or at least 97%, or at least 99% sequence identity with SEQ ID NO.: 4. Components (i) and (ii) may be linked directly or through a peptide linker, preferably through a peptide linker, such as a peptide linker as depicted in Table 4, e.g., a 9GS linker (SEQ ID NO.: 45) or a 35GS linker (SEQ ID NO.: 52).

In one embodiment, the polypeptide of the present technology of the present technology comprises two ISVDs, wherein each ISVD comprises or consists of SEQ ID NO.: 4, or a sequence which comprises or consists of a sequence with at least 90%, such as at least 95%, or at least 97%, or at least 99% sequence identity with SEQ ID NO.: 4. Hence, in one embodiment, the polypeptide of the present technology comprises as component (i) an ISVD comprising or consisting of SEQ ID NO.: 4, or a protein comprising or consisting of a sequence with at least 90%, such as at least 95%, or at least 97%, or at least 99% sequence identity with SEQ ID NO.: 4, and, as component (ii) an ISVDs comprising or consisting of SEQ ID NO.: 4, or a protein comprising or consisting of a sequence with at least 90%, such as at least 95%, or at least 97%, or at least 99% sequence identity with SEQ ID NO.: 4. Components (i) and (ii) may be linked directly or through a peptide linker, preferably through a peptide linker, such as a peptide linker as depicted in Table 4, e.g., a 35GS linker (SEQ ID NO.: 52) or a 9GS linker (SEQ ID NO.: 45).

Preferably, if the ISVD located at the C-terminal part of the polypeptide of the present technology it comprises a C-terminal A. For instance, if the ISVD is ALB23002 (SEQ ID NO.: 4) and it is located at the C-terminal of the polypeptide, the ISVD comprises a C-terminal A (i.e., the C-terminal ISVD would then be Alb223, SEQ ID NO.: 18). Hence, in one embodiment, the polypeptide of the present technology comprises as component (i) an ISVD comprising or consisting of SEQ ID NO.: 4, or a protein comprising or consisting of a sequence with at least 90%, such as at least 95%, or at least 97%, or at least 99% sequence identity with SEQ ID NO.: 4, and, as component (ii) an ISVDs comprising or consisting of SEQ ID NO.: 18, or a protein comprising or consisting of a sequence with at least 90%, such as at least 95%, or at least 97%, or at least 99% sequence identity with SEQ ID NO.: 18. Components (i) and (ii) may be linked directly or through a peptide linker, preferably through a peptide linker, such as a peptide linker as depicted in Table 4, e.g., a 35GS linker (SEQ ID NO.: 52) or a 9GS linker (SEQ ID NO.: 45).

In other embodiments, the polypeptide of the present technology comprises, as components (i) and (ii), two HSA proteins. The two HSA proteins may be the same or different. In one embodiment, the HSA proteins comprise or consist of SEQ ID NO.: 39 or 40, or a protein comprising or consisting of a sequence with at least 90%, such as at least 95%, or at least 97%, or at least 99% sequence identity with SEQ ID NO.: 39 or 40, preferably SEQ ID NO.: 40, or a protein comprising or consisting of a sequence with at least 90%, such as at least 95%, or at least 97%, or at least 99% sequence identity with SEQ ID NO.: 40. An example of polypeptides comprising two HSA as components (i) and (ii) is depicted Table 5 as SEQ ID NO.: 87.

The polypeptide of the present technology may also comprise one ISVD as defined herein and one HSA protein as defined herein.

In one embodiment, the polypeptide of the present technology comprises, as component (i), at least one immunoglobulin single variable domain (ISVD) which specifically binds to a serum albumin protein, wherein the at least one ISVD comprises three complementarity determining regions (CDR1 to CDR3, respectively), wherein:
a) CDR1 comprises the amino acid sequence of SEQ ID NO: 1 or has 3, 2 or 1 amino acid difference(s) with SEQ ID NO: 1 or CDR1 comprises the amino acid sequence of SEQ ID NO: 22 or has 3, 2 or 1 amino acid difference(s) with SEQ ID NO: 22
b) CDR2 comprises the amino acid sequence of SEQ ID NO: 2 or has 3, 2 or 1 amino acid difference(s) with SEQ ID NO: 2; and
c) CDR3 comprises the amino acid sequence of SEQ ID NO: 3 or has 3, 2 or 1 amino acid difference(s) with SEQ ID NO: 3,
   and/or
a) CDR1 comprises the amino acid sequence of SEQ ID NO: 36 or has 3, 2 or 1 amino acid difference(s) with SEQ ID NO: 36;
b) CDR2 comprises the amino acid sequence of SEQ ID NO: 37 or has 3, 2 or 1 amino acid difference(s) with SEQ ID NO: 37; and
c) CDR3 comprises the amino acid sequence of SEQ ID NO: 38 or has 3, 2 or 1 amino acid difference(s) with SEQ ID NO: 38,
   and/or
a) CDR1 comprises the amino acid sequence of SEQ ID NO: 55 or has 3, 2 or 1 amino acid difference(s) with SEQ ID NO: 55;
b) CDR2 comprises the amino acid sequence of SEQ ID NO: 56 or has 3, 2 or 1 amino acid difference(s) with SEQ ID NO: 56; and
c) CDR3 comprises the amino acid sequence of SEQ ID NO: 57 or has 3, 2 or 1 amino acid difference(s) with SEQ ID NO: 57,
   wherein the CDR sequences are determined according to AbM numbering,
   and, as component (ii), at least one further moiety specifically binding to a serum albumin protein, wherein the at least one further moiety specifically binding to a serum albumin protein is a DARPin, preferably a DARPin comprising or consisting of SEQ ID NO.: 88, or a protein comprising or consisting of a sequence with at least 90%, such as at least 95%, or at least 97%, or at least 99% sequence identity with SEQ ID NO.: 88.

For instance, in one embodiment, the polypeptide of the present technology comprises, as component (i), an ISVD comprising or consisting of SEQ ID NO.: 18, or a protein comprising or consisting of a sequence with at least 90%, such as at least 95%, or at least 97%, or at least 99% sequence identity with SEQ ID NO.: 18, and, as component (ii) a DARPin comprising or consisting of SEQ ID NO.: 88, or a protein comprising or consisting of a sequence with at least 90%, such as at least 95%, or at least 97%, or at least 99% sequence identity with SEQ ID NO.: 88. Components (i) and (ii) may be linked directly or through a peptide linker, preferably through a peptide linker, such as a peptide linker as depicted in Table 4, e.g., a 35GS linker (SEQ ID NO.: 52) or a 9GS linker (SEQ ID NO.: 45).

In another embodiment, the polypeptide of the present technology comprises, as component (i), an ISVD comprising or consisting of SEQ ID NO.: 4, or a protein comprising or consisting of a sequence with at least 90%, such as at least 95%, or at least 97%, or at least 99% sequence identity with SEQ ID NO.: 4, and, as component (ii) a DARPin comprising or consisting of SEQ ID NO.: 88, or a protein comprising or consisting of a sequence with at least 90%, such as at least 95%, or at least 97%, or at least 99% sequence identity with SEQ ID NO.: 88. Components (i) and (ii) may be linked directly or through a peptide linker, preferably through a peptide linker, such as a peptide linker as depicted in Table 4, e.g., a 35GS linker (SEQ ID NO.: 52) or a 9GS linker (SEQ ID NO.: 45).

An example of polypeptide comprising an ISVD as component (i) and a DARPin as component (ii) is depicted Table 5 as SEQ ID NO.: 79 or 150.

In another embodiment, the polypeptide of the present technology comprises, as component (i), at least one immunoglobulin single variable domain (ISVD) which specifically binds to a serum albumin protein, wherein the at least one ISVD comprises three complementarity determining regions (CDR1 to CDR3, respectively), wherein:
a) CDR1 comprises the amino acid sequence of SEQ ID NO: 1 or has 3, 2 or 1 amino acid difference(s) with SEQ ID NO: 1 or CDR1 comprises the amino acid sequence of SEQ ID NO: 22 or has 3, 2 or 1 amino acid difference(s) with SEQ ID NO: 22;
b) CDR2 comprises the amino acid sequence of SEQ ID NO: 2 or has 3, 2 or 1 amino acid difference(s) with SEQ ID NO: 2; and
c) CDR3 comprises the amino acid sequence of SEQ ID NO: 3 or has 3, 2 or 1 amino acid difference(s) with SEQ ID NO: 3,
   and/or
a) CDR1 comprises the amino acid sequence of SEQ ID NO: 36 or has 3, 2 or 1 amino acid difference(s) with SEQ ID NO: 36;
b) CDR2 comprises the amino acid sequence of SEQ ID NO: 37 or has 3, 2 or 1 amino acid difference(s) with SEQ ID NO: 37; and
c) CDR3 comprises the amino acid sequence of SEQ ID NO: 38 or has 3, 2 or 1 amino acid difference(s) with SEQ ID NO: 38,
   and/or
a) CDR1 comprises the amino acid sequence of SEQ ID NO: 55 or has 3, 2 or 1 amino acid difference(s) with SEQ ID NO: 55;
b) CDR2 comprises the amino acid sequence of SEQ ID NO: 56 or has 3, 2 or 1 amino acid difference(s) with SEQ ID NO: 56; and
c) CDR3 comprises the amino acid sequence of SEQ ID NO: 57 or has 3, 2 or 1 amino acid difference(s) with SEQ ID NO: 57,
   wherein the CDR sequences are determined according to AbM numbering,
   and, as component (ii), at least one further moiety specifically binding to a serum albumin protein, wherein the at least one further moiety specifically binding to a serum albumin protein is an Affitin, preferably an Affitin comprising or consisting of SEQ ID NO.: 89, or a protein comprising or consisting of a sequence with at least 90%, such as at least 95%, or at least 97%, or at least 99% sequence identity with SEQ ID NO.: 89.

For instance, in one embodiment, the polypeptide of the present technology comprises, as component (i), an ISVD comprising or consisting of SEQ ID NO.: 18, or a protein comprising or consisting of a sequence with at least 90%, such as at least 95%, or at least 97%, or at least 99% sequence identity with SEQ ID NO.: 18, and, as component (ii) an Affitin comprising or consisting of SEQ ID NO.: 89, or a protein comprising or consisting of a sequence with at least 90%, such as at least 95%, or at least 97%, or at least 99% sequence identity with SEQ ID NO.: 89. Components (i) and (ii) may be linked directly or through a peptide linker, preferably through a peptide linker, such as a peptide linker as depicted in Table 4, e.g., a 35GS linker (SEQ ID NO.: 52) or a 9GS linker (SEQ ID NO.: 45).

In another embodiment, the polypeptide of the present technology comprises, as component (i), an ISVD comprising or consisting of SEQ ID NO.: 4, or a protein comprising or consisting of a sequence with at least 90%, such as at least 95%, or at least 97%, or at least 99% sequence identity with SEQ ID NO.: 4, and, as component (ii) an Affitin comprising or consisting of SEQ ID NO.: 89, or a protein comprising or consisting of a sequence with at least 90%, such as at least 95%, or at least 97%, or at least 99% sequence identity with SEQ ID NO.: 89. Components (i) and (ii) may be linked directly or through a peptide linker, preferably through a peptide linker, such as a peptide linker as depicted in Table 4, e.g., a 35GS linker (SEQ ID NO.: 52) or a 9GS linker (SEQ ID NO.: 45).

An example of polypeptide comprising an ISVD as component (i) and a Affitin as component (ii) is depicted Table 5 as SEQ ID NO.: 80.

In another embodiment, the polypeptide of the present technology comprises, as component (i), at least one immunoglobulin single variable domain (ISVD) which specifically binds to a serum albumin protein, wherein the at least one ISVD comprises three complementarity determining regions (CDR1 to CDR3, respectively), wherein:
a) CDR1 comprises the amino acid sequence of SEQ ID NO: 1 or has 3, 2 or 1 amino acid difference(s) with SEQ ID NO: 1 or CDR1 comprises the amino acid sequence of SEQ ID NO: 22 or has 3, 2 or 1 amino acid difference(s) with SEQ ID NO: 22
b) CDR2 comprises the amino acid sequence of SEQ ID NO: 2 or has 3, 2 or 1 amino acid difference(s) with SEQ ID NO: 2; and
c) CDR3 comprises the amino acid sequence of SEQ ID NO: 3 or has 3, 2 or 1 amino acid difference(s) with SEQ ID NO: 3,
   and/or
a) CDR1 comprises the amino acid sequence of SEQ ID NO: 36 or has 3, 2 or 1 amino acid difference(s) with SEQ ID NO: 36;
b) CDR2 comprises the amino acid sequence of SEQ ID NO: 37 or has 3, 2 or 1 amino acid difference(s) with SEQ ID NO: 37; and
c) CDR3 comprises the amino acid sequence of SEQ ID NO: 38 or has 3, 2 or 1 amino acid difference(s) with SEQ ID NO: 38,
   and/or
a) CDR1 comprises the amino acid sequence of SEQ ID NO: 55 or has 3, 2 or 1 amino acid difference(s) with SEQ ID NO: 55;
b) CDR2 comprises the amino acid sequence of SEQ ID NO: 56 or has 3, 2 or 1 amino acid difference(s) with SEQ ID NO: 56; and
c) CDR3 comprises the amino acid sequence of SEQ ID NO: 57 or has 3, 2 or 1 amino acid difference(s) with SEQ ID NO: 57,
   wherein the CDR sequences are determined according to AbM numbering,
   and, as component (ii), at least one further moiety specifically binding to a serum albumin protein, wherein the at least one further moiety specifically binding to a serum albumin protein is an albumin binding domain (ABD), preferably an ABD comprising or consisting of SEQ ID NO.: 90, or a protein comprising or consisting of a sequence with at least 90%, such as at least 95%, or at least 97%, or at least 99% sequence identity with SEQ ID NO.: 90 or a 9GS linker (SEQ ID NO.: 45).

For instance, in one embodiment, the polypeptide of the present technology comprises, as component (i), an ISVD comprising or consisting of SEQ ID NO.: 18, or a protein comprising or consisting of a sequence with at least 90%, such as at least 95%, or at least 97%, or at least 99% sequence identity with SEQ ID NO.: 18, and, as component (ii) an albumin binding domain (ABD) comprising or consisting of SEQ ID NO.: 90, or a protein comprising or consisting of a sequence with at least 90%, such as at least 95%, or at least 97%, or at least 99% sequence identity with SEQ ID NO.: 90. Components (i) and (ii) may be linked directly or through a peptide linker, preferably through a peptide linker, such as a peptide linker as depicted in Table 4, e.g., a 35GS linker (SEQ ID NO.: 52) or a 9GS linker (SEQ ID NO.: 45).

In another embodiment, the polypeptide of the present technology comprises, as component (i), an ISVD comprising or consisting of SEQ ID NO.: 4, or a protein comprising or consisting of a sequence with at least 90%, such as at least 95%, or at least 97%, or at least 99% sequence identity with SEQ ID NO.: 4, and, as component (ii) an albumin binding domain (ABD) comprising or consisting of SEQ ID NO.: 90, or a protein comprising or consisting of a sequence with at least 90%, such as at least 95%, or at least 97%, or at least 99% sequence identity with SEQ ID NO.: 90. Components (i) and (ii) may be linked directly or through a peptide linker, preferably through a peptide linker, such as a peptide linker as depicted in Table 4, e.g., a 35GS linker (SEQ ID NO.: 52) or a 9GS linker (SEQ ID NO.: 45).

An example of polypeptide comprising an ISVD as component (i) and a ABD as component (ii) is depicted Table 5 as SEQ ID NO.: 81 or 151.

Hence, in a preferred embodiment, the polypeptide of the present technology comprises, as component (i), an ISVD comprising or consisting of SEQ ID NO.: 11 or a polypeptide comprising or consisting of a sequence with at least 90%, such as at least 95%, or at least 97%, or at least 99% sequence identity with SEQ ID NO.: 11 and, as component (ii), an ISVD comprising or consisting of SEQ ID NO.: 11 or a polypeptide comprising or consisting of a sequence with at least 90%, such as at least 95%, or at least 97%, or at least 99% sequence identity with SEQ ID NO.: 11.

In another preferred embodiment, the polypeptide of the present technology comprises, as component (i), an ISVD comprising or consisting of SEQ ID NO.: 54 or a polypeptide comprising or consisting of a sequence with at least 90%, such as at least 95%, or at least 97%, or at least 99% sequence identity with SEQ ID NO.: 54 and, as component (ii), an ISVD comprising or consisting of SEQ ID NO.: 18 or a polypeptide comprising or consisting of a sequence with at least 90%, such as at least 95%, or at least 97%, or at least 99% sequence identity with SEQ ID NO.: 18.

In another preferred embodiment, the polypeptide of the present technology comprises, as component (i), an ISVD comprising or consisting of SEQ ID NO.: 54 or a polypeptide comprising or consisting of a sequence with at least 90%, such as at least 95%, or at least 97%, or at least 99% sequence identity with SEQ ID NO.: 54 and, as component (ii), an ISVD comprising or consisting of SEQ ID NO.: 4 or a polypeptide comprising or consisting of a sequence with at least 90%, such as at least 95%, or at least 97%, or at least 99% sequence identity with SEQ ID NO.: 4.

In another preferred embodiment, the polypeptide of the present technology comprises, as component (i), an ISVD comprising or consisting of SEQ ID NO.: 20 or a polypeptide comprising or consisting of a sequence with at least 90%, such as at least 95%, or at least 97%, or at least 99% sequence identity with SEQ ID NO.: 20 and, as component (ii), an ISVD comprising or consisting of SEQ ID NO.: 18 or a polypeptide comprising or consisting of a sequence with at least 90%, such as at least 95%, or at least 97%, or at least 99% sequence identity with SEQ ID NO.: 18.

In another preferred embodiment, the polypeptide of the present technology comprises, as component (i), an ISVD comprising or consisting of SEQ ID NO.: 20 or a polypeptide comprising or consisting of a sequence with at least 90%, such as at least 95%, or at least 97%, or at least 99% sequence identity with SEQ ID NO.: 20 and, as component (ii), an ISVD comprising or consisting of SEQ ID NO.: 4 or a polypeptide comprising or consisting of a sequence with at least 90%, such as at least 95%, or at least 97%, or at least 99% sequence identity with SEQ ID NO.: 4.

In another preferred embodiment, the polypeptide of the present technology comprises, as component (i), an ISVD comprising or consisting of SEQ ID NO.: 91 or a polypeptide comprising or consisting of a sequence with at least 90%, such as at least 95%, or at least 97%, or at least 99% sequence identity with SEQ ID NO.: 91 and, as component (ii), an ISVD comprising or consisting of SEQ ID NO.: 92 or a polypeptide comprising or consisting of a sequence with at least 90%, such as at least 95%, or at least 97%, or at least 99% sequence identity with SEQ ID NO.: 92.

In another preferred embodiment, the polypeptide of the present technology comprises, as component (i), an ISVD comprising or consisting of SEQ ID NO.: 91 or a polypeptide comprising or consisting of a sequence with at least 90%, such as at least 95%, or at least 97%, or at least 99% sequence identity with SEQ ID NO.: 91 and, as component (ii), an ISVD comprising or consisting of SEQ ID NO.: 18 or a polypeptide comprising or consisting of a sequence with at least 90%, such as at least 95%, or at least 97%, or at least 99% sequence identity with SEQ ID NO.: 18.

In another preferred embodiment, the polypeptide of the present technology comprises, as component (i), an ISVD comprising or consisting of SEQ ID NO.: 91 or a polypeptide comprising or consisting of a sequence with at least 90%, such as at least 95%, or at least 97%, or at least 99% sequence identity with SEQ ID NO.: 91 and, as component (ii), an ISVD comprising or consisting of SEQ ID NO.: 4 or a polypeptide comprising or consisting of a sequence with at least 90%, such as at least 95%, or at least 97%, or at least 99% sequence identity with SEQ ID NO.: 4.

In another preferred embodiment, the polypeptide of the present technology comprises, as component (i), an ISVD comprising or consisting of SEQ ID NO.: 4 or a polypeptide comprising or consisting of a sequence with at least 90%, such as at least 95%, or at least 97%, or at least 99% sequence identity with SEQ ID NO.: 4 and, as component (ii), an ISVD comprising or consisting of SEQ ID NO.: 18 or a polypeptide comprising or consisting of a sequence with at least 90%, such as at least 95%, or at least 97%, or at least 99% sequence identity with SEQ ID NO.: 18.

In another preferred embodiment, the polypeptide of the present technology comprises, as component (i), an ISVD comprising or consisting of SEQ ID NO.: 4 or a polypeptide comprising or consisting of a sequence with at least 90%, such as at least 95%, or at least 97%, or at least 99% sequence identity with SEQ ID NO.: 4 and, as component (ii), an ISVD comprising or consisting of SEQ ID NO.: 4 or a polypeptide comprising or consisting of a sequence with at least 90%, such as at least 95%, or at least 97%, or at least 99% sequence identity with SEQ ID NO.: 4.

In another preferred embodiment, the polypeptide of the present technology comprises, as component (i), an ISVD comprising or consisting of SEQ ID NO.: 18 or a polypeptide comprising or consisting of a sequence with at least 90%, such as at least 95%, or at least 97%, or at least 99% sequence identity with SEQ ID NO.: 18 and, as component (ii), a DARPin comprising or consisting of SEQ ID NO.: 88 or a polypeptide comprising or consisting of a sequence with at least 90%, such as at least 95%, or at least 97%, or at least 99% sequence identity with SEQ ID NO.: 88.

In another preferred embodiment, the polypeptide of the present technology comprises, as component (i), an ISVD comprising or consisting of SEQ ID NO.: 4 or a polypeptide comprising or consisting of a sequence with at least 90%, such as at least 95%, or at least 97%, or at least 99% sequence identity with SEQ ID NO.: 4 and, as component (ii), a DARPin comprising or consisting of SEQ ID NO.: 88 or a polypeptide comprising or consisting of a sequence with at least 90%, such as at least 95%, or at least 97%, or at least 99% sequence identity with SEQ ID NO.: 88.

In another preferred embodiment, the polypeptide of the present technology comprises, as component (i), an ISVD comprising or consisting of SEQ ID NO.: 18 or a polypeptide comprising or consisting of a sequence with at least 90%, such as at least 95%, or at least 97%, or at least 99% sequence identity with SEQ ID NO.: 18 and, as component (ii), an Affitin comprising or consisting of SEQ ID NO.: 89 or a polypeptide comprising or consisting of a sequence with at least 90%, such as at least 95%, or at least 97%, or at least 99% sequence identity with SEQ ID NO.: 89.

In another preferred embodiment, the polypeptide of the present technology comprises, as component (i), an ISVD comprising or consisting of SEQ ID NO.: 4 or a polypeptide comprising or consisting of a sequence with at least 90%, such as at least 95%, or at least 97%, or at least 99% sequence identity with SEQ ID NO.: 4 and, as component (ii), an Affitin comprising or consisting of SEQ ID NO.: 89 or a polypeptide comprising or consisting of a sequence with at least 90%, such as at least 95%, or at least 97%, or at least 99% sequence identity with SEQ ID NO.: 89.

In another preferred embodiment, the polypeptide of the present technology comprises, as component (i), an ISVD comprising or consisting of SEQ ID NO.: 18 or a polypeptide comprising or consisting of a sequence with at least 90%, such as at least 95%, or at least 97%, or at least 99% sequence identity with SEQ ID NO.: 18 and, as component (ii), an albumin binding domain (ABD) comprising or consisting of SEQ ID NO.: 90 or a polypeptide comprising or consisting of a sequence with at least 90%, such as at least 95%, or at least 97%, or at least 99% sequence identity with SEQ ID NO.: 90.

In another preferred embodiment, the polypeptide of the present technology comprises, as component (i), an ISVD comprising or consisting of SEQ ID NO.: 4 or a polypeptide comprising or consisting of a sequence with at least 90%, such as at least 95%, or at least 97%, or at least 99% sequence identity with SEQ ID NO.: 4 and, as component (ii), an albumin binding domain (ABD) comprising or consisting of SEQ ID NO.: 90 or a polypeptide comprising or consisting of a sequence with at least 90%, such as at least 95%, or at least 97%, or at least 99% sequence identity with SEQ ID NO.: 90.

In another embodiment, the polypeptide of the present technology comprises, as component (i), a HSA comprising or consisting of SEQ ID NO.: 40 or a polypeptide comprising or consisting of a sequence with at least 90%, such as at least 95%, or at least 97%, or at least 99% sequence identity with SEQ ID NO.: 40 and, as component (ii), a HSA comprising or consisting of SEQ ID NO.: 40 or a polypeptide comprising or consisting of a sequence with at least 90%, such as at least 95%, or at least 97%, or at least 99% sequence identity with SEQ ID NO.: 40.

As explained above, preferably, components (i) and (ii) of the polypeptide of the present technology are linked by means of a peptide linker, such as the ones depicted in Table 4, preferably a 35GS linker (SEQ ID NO.: 52) or a 9GS linker (SEQ ID NO.: 45).

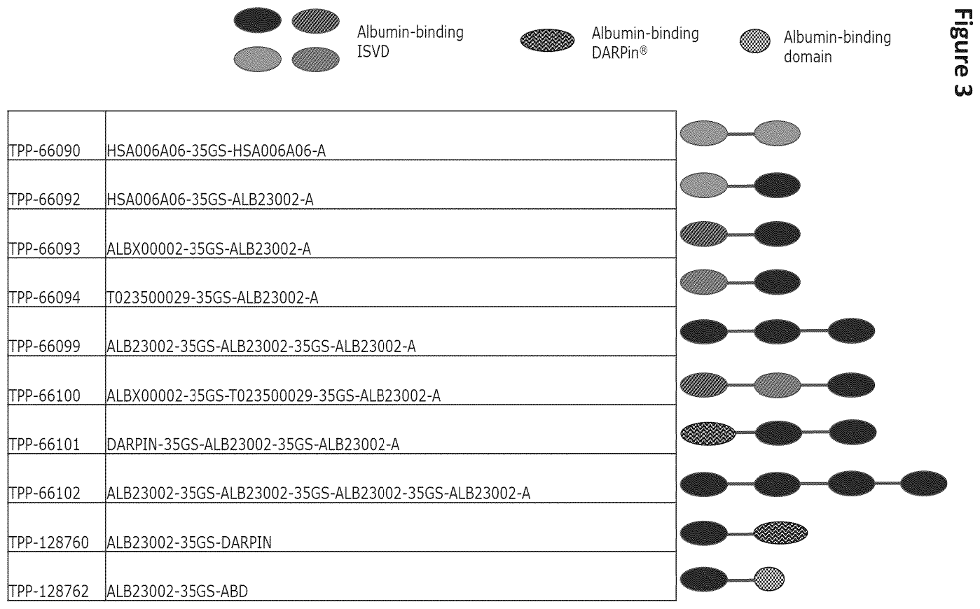

TABLE S
Examples of polypeptides of the present technology
(“ID” refers to the SEQ ID NO. as used herein)

| | | | Schematic |
|---|---|---|---|
| | | | drawing (see |
| Description | ID | Amino acid sequence | FIG. 3) |
| HSA006A | 74 | EVQLVESGGGLVQPGNSLRLSCAASGFTFRSFGMSWVRQAPGKE | [symbol] |
| 06-35GS- | | PEWVSSISGSGSDTLYADSVKGRFTISRDNAKTTLYLQMNSLKPEDT | |
| HSA006A | | AVYYCTIGGSLSRSSQGTQVTVSSGGGGSGGGGSGGGGSGGGGS | |
| 06-A | | GGGGSGGGGSGGGGSEVQLVESGGGLVQPGNSLRLSCAASGFTF | |
| | | RSFGMSWVRQAPGKEPEWVSSISGSGSDTLYADSVKGRFTISRDN | |
| | | AKTTLYLQMNSLKPEDTAVYYCTIGGSLSRSSQGTQVTVSSA | |
| ALB82- | 75 | EVQLVESGGGVVQPGNSLRLSCAASGFTFSSFGMSWVRQAPGKG | [symbol] |
| 35GS- | | LEWVSSISGSGSDTLYADSVKGRFTISRDNAKTTLYLQMNSLRPEDT | |
| ALB82-A | | ALYYCTIGGSLSRSSQGTLVTVSSGGGGSGGGGSGGGGSGGGGSG | |
| | | GGGSGGGGSGGGGSEVQLVESGGGVVQPGNSLRLSCAASGFTFS | |
| | | SFGMSWVRQAPGKGLEWVSSISGSGSDTLYADSVKGRFTISRDNA | |
| | | KTTLYLQMNSLRPEDTALYYCTIGGSLSRSSQGTLVTVSSA | |
| HSA006A | 76 | EVQLVESGGGLVQPGNSLRLSCAASGFTFRSFGMSWVRQAPGKE | [symbol] |
| 06-35GS- | | PEWVSSISGSGSDTLYADSVKGRFTISRDNAKTTLYLQMNSLKPEDT | |
| ALB23002 | | AVYYCTIGGSLSRSSQGTQVTVSSGGGGSGGGGSGGGGSGGGGS | |
| -A | | GGGGSGGGGSGGGGSEVQLVESGGGVVQPGGSLRLSCAASGFTF | |
| (HSA006A | | RSFGMSWVRQAPGKGPEWVSSISGSGSDTLYADSVKGRFTISRDN | |
| 06-35GS- | | SKNTLYLQMNSLRPEDTALYYCTIGGSLSRSSQGTLVTVSSA | |
| Alb223) | | | |
| ALBX0000 | 77 | EVQLVESGGGVVQPGGSLRLSCAASGLTFSSYAMGWFRQAPGKE | [symbol] |
| 2-35GS- | | RERVVSISRGGGYTYYADSVKGRFTISRDNSKNTVYLQMNSLRPED | |
| ALB23002 | | TALYYCAAARYWATGSEYEFDYWGQGTLVTVSSGGGGSGGGGSG | |
| -A | | GGGSGGGGSGGGGSGGGGSGGGGSEVQLVESGGGVVQPGGSL | |
| (ALBX000 | | RLSCAASGFTFRSFGMSWVRQAPGKGPEWVSSISGSGSDTLYADS | |
| 02-35GS- | | VKGRFTISRDNSKNTLYLQMNSLRPEDTALYYCTIGGSLSRSSQGTL | |
| Alb223) | | VTVSSA | |
| T0235000 | 78 | EVQLVESGGGVVQPGDSLRLSCAASGGTFSTYVMGWFRQAPGKE | [symbol] |
| 29-35GS- | | REFVSAISQNSIHTYYANSVKGRFTISRDNSKNTVYLQLNSLRPEDTA | |
| ALB23002 | | LYYCAASRFTSWYTADYEYDYWGQGTLVTVSSGGGGSGGGGSGG | |
| -A | | GGSGGGGSGGGGSGGGGSGGGGSEVQLVESGGGVVQPGGSLRL | |
| (T023500 | | SCAASGFTFRSFGMSWVRQAPGKGPEWVSSISGSGSDTLYADSVK | |
| 029- | | GRFTISRDNSKNTLYLQMNSLRPEDTALYYCTIGGSLSRSSQGTLVT | |
| 35GS- | | VSSA | |
| Alb223) | | | |
| DARPIN- | 79 | DLGKKLLEAARAGQDDEVRELLKAGADVNAKDYFSHTPLHLAARN | [symbol] |
| 35GS- | | GHLKIVEVLLKAGADVNAKDFAGKTPLHLAANEGHLEIVEVLLKAG | |
| ALB23002 | | ADVNAQDIFGKTPADIAADAGHEDIAEVLQKAAGGGGSGGGGSG | |
| -A | | GGGSGGGGSGGGGSGGGGSGGGGSEVQLVESGGGVVQPGGSL | |
| (DARPIN- | | RLSCAASGFTFRSFGMSWVRQAPGKGPEWVSSISGSGSDTLYADS | |
| 35GS- | | VKGRFTISRDNSKNTLYLQMNSLRPEDTALYYCTIGGSLSRSSQGTL | |
| Alb223) | | VTVSSA | |
| Nanofitin- | 80 | VKVKFWPRGEEKVVDTSKIAWVLRADKTVMFKYDDNGKKGYGVV | [symbol] |
| 35GS- | | LEKDAPKELLDMLARAEREKGGGGSGGGGSGGGGSGGGGSGGG | |
| ALB23002 | | GSGGGGSGGGGSEVQLVESGGGVVQPGGSLRLSCAASGFTFRSFG | |
| -A | | MSWVRQAPGKGPEWVSSISGSGSDTLYADSVKGRFTISRDNSKNT | |
| (Nanofitin | | LYLQMNSLRPEDTALYYCTIGGSLSRSSQGTLVTVSSA | |
| -35GS- | | | |
| Alb223) | | | |
| ABD- | 81 | LKEAKEKAIEELKKAGITSDYYFDLINKAKTVEGVNALKDEILKAGGG | [symbol] |
| 35GS- | | GSGGGGSGGGGSGGGGSGGGGSGGGGSGGGGSEVQLVESGGG | |
| ALB23002 | | VVQPGGSLRLSCAASGFTFRSFGMSWVRQAPGKGPEWVSSISGS | |
| -A (ABD- | | GSDTLYADSVKGRFTISRDNSKNTLYLQMNSLRPEDTALYYCTIGGS | |
| 35GS- | | LSRSSQGTLVTVSSA | |
| Alb223) | | | |
| HSA (25-609)- | 87 | DAHKSEVAHRFKDLGEENFKALVLIAFAQYLQQCPFEDHVKLVNEV TEFAKTCVADESAENCDKSLHTLFGDKLCTVATLRETYGEMADCCA | [symbol] |
| 35GS- | | KQEPERNECFLQHKDDNPNLPRLVRPEVDVMCTAFHDNEETFLKKY | |
| HSA(25- | | LYEIARRHPYFYAPELLFFAKRYKAAFTECCQAADKAACLLPKLDE | |
| 609) | | LRDEGKASSAKQRLKCASLQKFGERAFKAWAVARLSQRFPKAEFAE | |
| | | VSKLVTDLTKVHTECCHGDLLECADDRADLAKYICENQDSISSKLK | |
| | | ECCEKPLLEKSHCIAEVENDEMPADLPSLAADFVESKDVCKNYAEAK | |
| | | DVFLGMFLYEYARRHPDYSVVLLLRLAKTYETTLEKCCAAADPHECY | |
| | | AKVFDEFKPLVEEPQNLIKQNCELFEQLGEYKFQNALLVRYTKKVPQ | |
| | | VSTPTLVEVSRNLGKVGSKCCKHPEAKRMPCAEDYLSVVLNQLCVL | |
| | | HEKTPVSDRVTKCCTESLVNRRPCFSALEVDETYVPKEFNAETFTFH | |
| | | ADICTLSEKERQIKKQTALVELVKHKPKATKEQLKAVMDDFAAFVE | |
| | | KCCKADDKETCFAEEGKKLVAASQAALGLGGGGSGGGGSGGGGS | |
| | | GGGGSGGGGSGGGGSGGGGSDAHKSEVAHRFKDLGEENFKALVLI | |
| | | AFAQYLQQCPFEDHVKLVNEVTEFAKTCVADESAENCDKSLHTLF | |
| | | GDKLCTVATLRETYGEMADCCAKQEPERNECFLQHKDDNPNLPRL | |
| | | VRPEVDVMCTAFHDNEETFLKKYLYEIARRHPYFYAPELLFFAKRY | |
| | | KAAFTECCQAADKAACLLPKLDELRDEGKASSAKQRLKCASLQKFG | |
| | | ERAFKAWAVARLSQRFPKAEFAEVSKLVTDLTKVHTECCHGDLLEC | |
| | | ADDRADLAKYICENQDSISSKLKECCEKPLLEKSHCIAEVENDEMP | |
| | | ADLPSLAADFVESKDVCKNYAEAKDVFLGMFLYEYARRHPDYSVVL | |
| | | LLRLAKTYETTLEKCCAAADPHECYAKVFDEFKPLVEEPQNLIKQN | |
| | | CELFEQLGEYKFQNALLVRYTKKVPQVSTPTLVEVSRNLGKVGSKC | |
| | | CKHPEAKRMPCAEDYLSVVLNQLCVLHEKTPVSDRVTKCCTESLVN | |
| | | CFSALEVDETYVPKEFNAETFTFHADICTLSEKERQIKKQTALVELV | |
| | | RRPKHKPKATKEQLKAVMDDFAAFVEKCCKADDKETCFAEEGKKLVAAS | |
| | | QAALGL | |
| ALB23002 | 147 | EVQLVESGGGVVQPGGSLRLSCAASGFTFRSFGMSWVRQAPGKG | [symbol] |
| -35GS- | | PEWVSSISGSGSDTLYADSVKGRFTISRDNSKNTLYLQMNSLRPED | |
| ALB23002 | | TALYYCTIGGSLSRSSQGTLVTVSSGGGGSGGGGSGGGGSGGGGS | |
| -A | | GGGGSGGGGSGGGGSEVQLVESGGGVVQPGGSLRLSCAASGFTF | |
| (ALB2300 | | RSFGMSWVRQAPGKGPEWVSSISGSGSDTLYADSVKGRFTISRDN | |
| 2-35GS- | | SKNTLYLQMNSLRPEDTALYYCTIGGSLSRSSQGTLVTVSSA | |
| Alb223) | | | |
| ALB23002 | 152 | EVQLVESGGGVVQPGGSLRLSCAASGFTFRSFGMSWVRQAPGKG | [symbol] |
| -35GS- | | PEWVSSISGSGSDTLYADSVKGRFTISRDNSKNTLYLQMNSLRPED | |
| ALB23002 | | TALYYCTIGGSLSRSSQGTLVTVSSGGGGSGGGGSGGGGSGGGGS | |
| | | GGGGGGGGSGGGGSEVQLVESGGGVVQPGGSLRLSCAASGFTF | |
| | | RSFGMSWVRQAPGKGPEWVSSISGSGSDTLYADSVKGRFTISRDN | |
| | | SKNTLYLQMNSLRPEDTALYYCTIGGSLSRSSQGTLVTVSS | |
| ALB23002 | 153 | EVQLVESGGGVVQPGGSLRLSCAASGFTFRSFGMSWVRQAPGKG | [symbol] |
| -20GS- | | PEWVSSISGSGSDTLYADSVKGRFTISRDNSKNTLYLQMNSLRPED | |
| ALB23002 | | TALYYCTIGGSLSRSSQGTLVTVSSGGGGSGGGGSGGGGSGGGGS | |
| | | EVQLVESGGGVVQPGGSLRLSCAASGFTFRSFGMSWVRQAPGKG | |
| | | PEWVSSISGSGSDTLYADSVKGRFTISRDNSKNTLYLQMNSLRPED | |
| | | TALYYCTIGGSLSRSSQGTLVTVSS | |
| ALB23002 | 154 | EVQLVESGGGVVQPGGSLRLSCAASGFTFRSFGMSWVRQAPGKG | [symbol] |
| -9GS- | | PEWVSSISGSGSDTLYADSVKGRFTISRDNSKNTLYLQMNSLRPED | |
| ALB23002 | | TALYYCTIGGSLSRSSQGTLVTVSSGGGGSGGGSEVQLVESGGGVV | |
| | | QPGGSLRLSCAASGFTFRSFGMSWVRQAPGKGPEWVSSISGSGSD | |
| | | TLYADSVKGRFTISRDNSKNTLYLQMNSLRPEDTALYYCTIGGSLSRS | |
| | | SQGTLVTVSS | |
| ALB23002 | 148 | EVQLVESGGGVVQPGGSLRLSCAASGFTFRSFGMSWVRQAPGKG | [symbol] |
| -9GS- | | PEWVSSISGSGSDTLYADSVKGRFTISRDNSKNTLYLQMNSLRPED | |
| ALB23002-A | | TALYYCTIGGSLSRSSQGTLVTVSSGGGGSGGGSEVQLVESGGGVV | |
| (ALB2300 | | QPGGSLRLSCAASGFTFRSFGMSWVRQAPGKGPEWVSSISGSGSD | |
| 2-9GS- | | TLYADSVKGRFTISRDNSKNTLYLQMNSLRPEDTALYYCTIGGSLSRS | |
| Alb223) | | SQGTLVTVSSA | |
| ALB23002 | 149 | EVQLVESGGGVVQPGGSLRLSCAASGFTFRSFGMSWVRQAPGKG | [symbol] |
| -20GS- | | PEWVSSISGSGSDTLYADSVKGRFTISRDNSKNTLYLQMNSLRPED | |
| ALB23002 | | TALYYCTIGGSLSRSSQGTLVTVSSGGGGSGGGGSGGGGSGGGGS | |
| -A | | EVQLVESGGGVVQPGGSLRLSCAASGFTFRSFGMSWVRQAPGKG | |
| (ALB2300 | | PEWVSSISGSGSDTLYADSVKGRFTISRDNSKNTLYLQMNSLRPED | |
| 2-20GS- | | TALYYCTIGGSLSRSSQGTLVTVSSA | |
| Alb223) | | | |
| ALB23002 | 150 | EVQLVESGGGVVQPGGSLRLSCAASGFTFRSFGMSWVRQAPGKG | [symbol] |
| -35GS- | | PEWVSSISGSGSDTLYADSVKGRFTISRDNSKNTLYLQMNSLRPED | |
| DARPIN | | TALYYCTIGGSLSRSSQGTLVTVSSGGGGSGGGGSGGGGSGGGGS | |
| | | GGGGSGGGGSGGGGSDLGKKLLEAARAGQDDEVRELLKAGADV | |
| | | NAKDYFSHTPLHLAARNGHLKIVEVLLKAGADVNAKDFAGKTPLHL | |
| | | AANEGHLEIVEVLLKAGADVNAQDIFGKTPADIAADAGHEDIAEVL | |
| | | QKAA | |
| ALB23002 | 151 | EVQLVESGGGVVQPGGSLRLSCAASGFTFRSFGMSWVRQAPGKG | [symbol] |
| -35GS- | | PEWVSSISGSGSDTLYADSVKGRFTISRDNSKNTLYLQMNSLRPED | |
| ABD | | TALYYCTIGGSLSRSSQGTLVTVSSGGGGSGGGGSGGGGSGGGGS | |
| | | GGGGSGGGGSGGGGSLKEAKEKAIEELKKAGITSDYYFDLINKAKT | |
| | | VEGVNALKDEILKA | |

In one embodiment, the polypeptide of the present technology comprises:
(i) at least one immunoglobulin single variable domain (ISVD) which specifically binds to a serum albumin protein as defined herein, wherein the ISVD is preferably not an ISVD comprising or consisting of SEQ ID NO.: 4 or SEQ ID NO.: 18; and
(ii) at least one human serum albumin protein.

The present technology thus provides a polypeptide which comprises or consist of a sequence selected from SEQ ID NO.: 74-81, 87 and 147-152, or a sequence which comprises or consists of a sequence with at least 90%, such as at least 95%, or at least 97%, or at least 99% sequence identity with a sequence selected from SEQ ID NO.: 74-81, 87 and 147-154.

### Fusion Protein

The present technology further provides a fusion protein or construct comprising the polypeptide of the present technology. The fusion protein of the present technology may comprise, besides the polypeptide, further groups, residues, moieties or binding units. As will become clear to the skilled person from the further disclosure herein, such further groups, residues, moieties, binding units or amino acid sequences may or may not provide further functionality to the polypeptide of the technology and may or may not modify the properties of the polypeptide of the technology.

The additional one or more further groups, residues, moieties or binding units comprised in the fusion protein of the present technology may be directly linked to the polypeptide of the present technology (as for example described in WO 99/23221) and/or via one or more suitable spacers or linkers, or any combination thereof.

Suitable spacers or linkers for use in the fusion protein of the present technology will be clear to the skilled person and may generally be any linker or spacer used in the art to link amino acid sequences. Preferably, said linker or spacer is suitable for use in constructing proteins or polypeptides that are intended for pharmaceutical use. The linkers for use in the fusion protein of the present technology may be the same as the ones used for linking components (i) and (ii) of the polypeptide of the present technology, described above. Preferred linkers are depicted in Table 4, in particular 9GS, 20GS and 35GS, SEQ ID NO.: 45, 49 or 52, more preferably 9GS and 35GS, even more preferably 35GS.

It will be appreciated that the order of the polypeptide and further groups, residues, moieties or binding units, if present, in the fusion protein of the technology can be chosen according to the needs of the person skilled in the art, as well as the relative affinities which may depend on the location of the polypeptide and further groups, residues, moieties or binding units, if present, in the fusion protein. Whether the fusion protein comprises one or more linkers to interconnect the polypeptide and optionally further groups, residues, moieties or binding units is a matter of design choice. However, some orientations, with or without linkers, may provide preferred binding characteristics in comparison to other orientations. All different possible orientations are encompassed by the technology.

When two or more linkers are used in the polypeptides of the technology, these linkers may be the same or different. Again, based on the disclosure herein, the skilled person will be able to determine the optimal linkers for use in a specific fusion protein of the technology, optionally after some limited routine experiments.

Usually, for ease of expression and production, a fusion protein of the technology will be a linear protein. However, the technology in its broadest sense is not limited thereto. For example, when a fusion protein of the technology comprises three or more domains (e.g., at least two domains comprised in the polypeptide as components (i) and (ii) and at least one further domain to form the fusion protein), it is possible to link them by use of a linker with three or more “arms”, with each “arm” being linked to a domain, so as to provide a “star-shaped” construct. It is also possible, although usually less preferred, to use circular constructs.

Such further groups, residues, moieties or binding units comprised in the fusion protein of the present technology may be one or more additional immunoglobulins, so as to form a (fusion) protein (the fusion protein of the present technology). In a preferred but non-limiting aspect, the one or more other groups, residues, moieties or binding units are ISVDs. Even more preferably, the one or more other groups, residues, moieties or binding units are chosen from the group consisting of domain antibodies, ISVDs that are suitable for use as a domain antibody, single domain antibodies, ISVDs that are suitable for use as a single domain antibody, “dAb”'s, ISVDs that are suitable for use as a dAb, V_{HH}s, humanized V_{HH}s, camelized V_{H}s, or Nanobody® V_{HH}s. Preferably, the ISVD is derived from V_{H} or V_{HH}, more preferably the ISVD is a single domain antibody (dAb), even more preferably a Nanobody ISVD. For instance, in the context of the present technology, the further groups, residues, moieties or binding units that are comprised in the fusion protein of the present technology together with the polypeptide of the present technology are one or more ISVDs as described in this application the context of the polypeptide of the present technology.

The at least one further group comprised in the fusion protein of the present technology may be any of the albumin binders described in the present description under section “first domain (i)” and “second domain (ii)”.

Hence, in one embodiment, the fusion protein of the present technology comprises the polypeptide of the present technology and at least one further group, residue, moiety or binding unit, wherein the at least one further group, residue, moiety or binding unit is an ISVD which comprises three complementarity determining regions (CDR1 to CDR3, respectively), wherein:
a) CDR1 comprises the amino acid sequence of SEQ ID NO: 1 or has 3, 2 or 1 amino acid difference(s) with SEQ ID NO: 1 or CDR1 comprises the amino acid sequence of SEQ ID NO: 22 or has 3, 2 or 1 amino acid difference(s) with SEQ ID NO: 22
b) CDR2 comprises the amino acid sequence of SEQ ID NO: 2 or has 3, 2 or 1 amino acid difference(s) with SEQ ID NO: 2; and
c) CDR3 comprises the amino acid sequence of SEQ ID NO: 3 or has 3, 2 or 1 amino acid difference(s) with SEQ ID NO: 3,
   and/or
a) CDR1 comprises the amino acid sequence of SEQ ID NO: 36 or has 3, 2 or 1 amino acid difference(s) with SEQ ID NO: 36;
b) CDR2 comprises the amino acid sequence of SEQ ID NO: 37 or has 3, 2 or 1 amino acid difference(s) with SEQ ID NO: 37; and
c) CDR3 comprises the amino acid sequence of SEQ ID NO: 38 or has 3, 2 or 1 amino acid difference(s) with SEQ ID NO: 38,
   and/or
a) CDR1 comprises the amino acid sequence of SEQ ID NO: 55 or has 3, 2 or 1 amino acid difference(s) with SEQ ID NO: 55;
b) CDR2 comprises the amino acid sequence of SEQ ID NO: 56 or has 3, 2 or 1 amino acid difference(s) with SEQ ID NO: 56; and
c) CDR3 comprises the amino acid sequence of SEQ ID NO: 57 or has 3, 2 or 1 amino acid difference(s) with SEQ ID NO: 57,
   wherein the CDR sequences are determined according to AbM numbering.

In one embodiment, the further group, residue, moiety or binding unit comprised in the fusion protein of the present technology is an ISVD which comprises or consists of a sequence selected from SEQ ID NO.: 4 to 21, 54, or 91 to 93, or a sequence which comprises or consists of a sequence with at least 90%, such as at least 95%, or at least 97%, or at least 99% sequence identity with a sequence selected from SEQ ID NO.: 4 to 21, 54, or 91 to 93.

In another embodiment, the further group, residue, moiety or binding unit comprised in the fusion protein of the present technology is an ISVD which comprises or consists of a sequence selected from SEQ ID NO.: 4 and 18, or a sequence which comprises or consists of a sequence with at least 90%, such as at least 95%, or at least 97%, or at least 99% sequence identity with a sequence selected from SEQ ID NO.: 4 and 18. If the further group, residue, moiety or binding unit comprised in the fusion protein of the present technology is located at the C-terminal of the fusion protein, the further group, residue, moiety or binding unit comprised in the fusion protein of the present technology may have a C-terminal alanine. For instance, if the further group, residue, moiety or binding unit comprised in the fusion protein of the present technology is an ISVD which comprises or consists of SEQ ID NO.: 4, it may comprise a C-terminal alanine (SEQ ID NO.: 18).

In one embodiment, the further group, residue, moiety or binding unit comprised in the fusion protein of the present technology is an ISVD which comprises or consists of SEQ ID NO.: 20, or a sequence which comprises or consists of a sequence with at least 90%, such as at least 95%, or at least 97%, or at least 99% sequence identity with SEQ ID NO.: 20.

In another embodiment, the further group, residue, moiety or binding unit comprised in the fusion protein of the present technology is an ISVD which comprises or consists of SEQ ID NO.: 54, or a sequence which comprises or consists of a sequence with at least 90%, such as at least 95%, or at least 97%, or at least 99% sequence identity with SEQ ID NO.: 54.

In another embodiment, the further group, residue, moiety or binding unit comprised in the fusion protein of the present technology is an ISVD which comprises or consists of SEQ ID NO.: 91, or a sequence which comprises or consists of a sequence with at least 90%, such as at least 95%, or at least 97%, or at least 99% sequence identity with SEQ ID NO.: 91.

In another embodiment, the further group, residue, moiety or binding unit comprised in the fusion protein of the present technology is an ISVD which comprises or consists of SEQ ID NO.: 92, or a sequence which comprises or consists of a sequence with at least 90%, such as at least 95%, or at least 97%, or at least 99% sequence identity with SEQ ID NO.: 92.

The further group, residue, moiety or binding unit comprised in the fusion protein of the present technology may also be a human serum albumin protein, for example as the ones depicted in SEQ ID NO.: 39 or 40, preferably SEQ ID NO.: 40, or a HSA protein which comprises or consists of a sequence with at least 90%, such as at least 95%, or at least 97%, or at least 99% sequence identity with SEQ ID NO.: 39 or 40, preferably with SEQ ID NO.: 40.

In one embodiment, the further group, residue, moiety or binding unit comprised in the fusion protein of the present technology is a DARPin, an Affitin or an albumin binding domain (ABD), as described above. If this is the case, the further group, residue, moiety or binding unit comprised in the fusion protein of the present technology may be a protein which comprises or consists of a sequence selected from SEQ ID NO.: 88-90, or a sequence which comprises or consists of a sequence with at least 90%, such as at least 95%, or at least 97%, or at least 99% sequence identity with SEQ ID NO.: 88-90.

Hence, in one embodiment, the further group, residue, moiety or binding unit comprised in the fusion protein of the present technology is a DARPin which comprises or consists of SEQ ID NO.: 88, or a sequence which comprises or consists of a sequence with at least 90%, such as at least 95%, or at least 97%, or at least 99% sequence identity with SEQ ID NO.: 88.

In another embodiment, the further group, residue, moiety or binding unit comprised in the fusion protein of the present technology is an Affitin which comprises or consists of SEQ ID NO.: 89, or a sequence which comprises or consists of a sequence with at least 90%, such as at least 95%, or at least 97%, or at least 99% sequence identity with SEQ ID NO.: 89.

In another embodiment, the further group, residue, moiety or binding unit comprised in the fusion protein of the present technology is an ABD which comprises or consists of SEQ ID NO.: 90, or a sequence which comprises or consists of a sequence with at least 90%, such as at least 95%, or at least 97%, or at least 99% sequence identity with SEQ ID NO.: 90.

In one embodiment, the fusion protein of the present technology comprises three ISVDs, wherein each ISVD comprises or consists of SEQ ID NO.: 4, or a sequence which comprises or consists of a sequence with at least 90%, such as at least 95%, or at least 97%, or at least 99% sequence identity with SEQ ID NO.: 4. Preferably, the three ISVDs are linked directly or through a peptide linker, preferably through a peptide linker, such as a peptide linker as depicted in Table 4, e.g., a 35GS linker (SEQ ID NO.: 52) or a 9GS linker (SEQ ID NO.: 45). Hence, in a preferred embodiment, the fusion protein comprises three ISVDs comprising or consisting of SEQ ID NO.: 4 and two peptide linkers (preferably as depicted in SEQ ID NO.: 52 or SEQ ID NO.: 45, even more preferably as depicted in SEQ ID NO.: 52) which link the ISVDs to each other. Preferably, the ISVD located at the C-terminal part of the fusion protein comprises a C-terminal A. If the C-terminal ISVD is Alb2003 (SEQ ID NO.: 4), since it comprises a C-terminal A, the ISVD would comprise or consist of SEQ ID NO.: 18.

In another embodiment, the fusion protein of the present technology comprises a polypeptide comprising two ISVDs comprising or consisting of SEQ ID NO.: 4, or a sequence which comprises or consists of a sequence with at least 90%, such as at least 95%, or at least 97%, or at least 99% sequence identity with SEQ ID NO.: 4 and one further moiety, which is an ISVD which comprises or consists of SEQ ID NO.: 18, or a sequence which comprises or consists of a sequence with at least 90%, such as at least 95%, or at least 97%, or at least 99% sequence identity with SEQ ID NO.: 18. Preferably, the three ISVDs are linked directly or through a peptide linker, preferably through a peptide linker, such as a peptide linker as depicted in Table 4, e.g., a 35GS linker (SEQ ID NO.: 52) or a 9GS linker (SEQ ID NO.: 45). Hence, in a preferred embodiment, the fusion protein comprises or consists of three ISVDs, two of them comprising or consisting of SEQ ID NO.: 4 and one of them comprising or consisting of SEQ ID NO.: 18 and two peptide linkers (preferably as depicted in SEQ ID NO.: 52 or SEQ ID NO.: 45) which link the ISVDs to each other. Preferably, the ISVD located at the C-terminal part of the fusion protein comprises a C-terminal A (SEQ ID NO.: 18). For instance, in this embodiment, the fusion protein may comprise or consist of SEQ ID NO.: 82 or a protein which comprises or consists of a sequence with at least 90%, such as at least 95%, or at least 97%, or at least 99% sequence identity with SEQ ID NO.: 82. For instance, in this embodiment, the fusion protein may comprise or consist of SEQ ID NO.: 85 or a protein which comprises or consists of a sequence with at least 90%, such as at least 95%, or at least 97%, or at least 99% sequence identity with SEQ ID NO.: 85.

In another embodiment, the fusion protein of the present technology comprises a polypeptide comprising two ISVDs, one comprising or consisting of SEQ ID NO.: 54, or a sequence which comprises or consists of a sequence with at least 90%, such as at least 95%, or at least 97%, or at least 99% sequence identity with SEQ ID NO.: 54 and the other one comprising or consisting of SEQ ID NO.: 20, or a sequence which comprises or consists of a sequence with at least 90%, such as at least 95%, or at least 97%, or at least 99% sequence identity with SEQ ID NO.: 20, and one further moiety, which is an ISVD which comprises or consists of SEQ ID NO.: 18, or a sequence which comprises or consists of a sequence with at least 90%, such as at least 95%, or at least 97%, or at least 99% sequence identity with SEQ ID NO.: 18. Preferably, the three ISVDs are linked directly or through a peptide linker, preferably through a peptide linker, such as a peptide linker as depicted in Table 4, e.g., a 35GS linker (SEQ ID NO.: 52) or a 9GS liner (SEQ ID NO.: 45). Hence, in a preferred embodiment, the fusion protein comprises three ISVDs, one of them comprising or consisting of SEQ ID NO.: 54, another one comprising or consisting of SEQ ID NO.: 20, another one comprising or consisting of SEQ ID NO.: 18, and two peptide linkers (preferably as depicted in SEQ ID NO.: 52 or SEQ ID NO.: 45), which link the ISVDs to each other. Preferably, the ISVD located at the C-terminal part of the fusion protein comprises a C-terminal A (SEQ ID NO.: 18). For instance, in this embodiment, the fusion protein may comprise or consist of SEQ ID NO.: 83 or a protein which comprises or consists of a sequence with at least 90%, such as at least 95%, or at least 97%, or at least 99% sequence identity with SEQ ID NO.: 83.

In another embodiment, the fusion protein of the present technology comprises a polypeptide comprising one ISVD comprising or consisting of SEQ ID NO.: 4, or a sequence which comprises or consists of a sequence with at least 90%, such as at least 95%, or at least 97%, or at least 99% sequence identity with SEQ ID NO.: 4 and one DARPin comprising or consisting of SEQ ID NO.: 88, or a sequence which comprises or consists of a sequence with at least 90%, such as at least 95%, or at least 97%, or at least 99% sequence identity with SEQ ID NO.: 88, and one further moiety, which is an ISVD which comprises or consists of SEQ ID NO.: 18, or a sequence which comprises or consists of a sequence with at least 90%, such as at least 95%, or at least 97%, or at least 99% sequence identity with SEQ ID NO.: 18. Preferably, the two ISVDs and the DARPin are linked directly or through a peptide linker, preferably through a peptide linker, such as a peptide linker as depicted in Table 4, e.g., a 35GS linker (SEQ ID NO.: 52) or a 9GS linker (SEQ ID NO.: 45). Hence, in one embodiment, the fusion protein comprises two ISVDs comprising or consisting of SEQ ID NO.: 4 and DARPin comprising or consisting of SEQ ID NO.: 88 and two peptide linkers (preferably as depicted in SEQ ID NO.: 52 or SEQ ID NO.: 45) which link the ISVDs to each other and to the DARPin. Preferably, the ISVD located at the C-terminal part of the fusion protein comprises a C-terminal A (SEQ ID NO.: 18). Hence, in a preferred embodiment, the fusion protein comprises two ISVDs, one of them comprising or consisting of SEQ ID NO.: 4, another one comprising or consisting of SEQ ID NO.: 18, one DARPin comprising or consisting of SEQ ID NO.: 88 and two peptide linkers (preferably as depicted in SEQ ID NO.: 52 or SEQ ID NO.: 45) which link the ISVDs to each other and to the DARPin. The ISVD located at the C-terminal part of the fusion protein preferably comprises a C-terminal A (SEQ ID NO.: 18). For instance, in this embodiment, the fusion protein may comprise or consist of SEQ ID NO.: 84 or a protein which comprises or consists of a sequence with at least 90%, such as at least 95%, or at least 97%, or at least 99% sequence identity with SEQ ID NO.: 84.

In another embodiment, the fusion protein of the present technology comprises a polypeptide comprising two ISVDs comprising or consisting of SEQ ID NO.: 4, or a sequence which comprises or consists of a sequence with at least 90%, such as at least 95%, or at least 97%, or at least 99% sequence identity with SEQ ID NO.: 4 and two further moieties, which are two ISVDs, one comprising or consisting of SEQ ID NO.: 4, or a sequence which comprises or consists of a sequence with at least 90%, such as at least 95%, or at least 97%, or at least 99% sequence identity with SEQ ID NO.: 4 and another one comprising or consisting of SEQ ID NO.: 18, or a sequence which comprises or consists of a sequence with at least 90%, such as at least 95%, or at least 97%, or at least 99% sequence identity with SEQ ID NO.: 18. Preferably, the four ISVDs are linked directly or through a peptide linker, preferably through a peptide linker, such as a peptide linker as depicted in Table 4, e.g., a 35GS linker (SEQ ID NO.: 52) or a 9GS linker (SEQ ID NO.: 45). In one embodiment, the fusion protein comprises four ISVDs as described above, and two peptide linkers (preferably as depicted in SEQ ID NO.: 52 or SEQ ID NO.: 45) which link the ISVDs to each other. Preferably, the ISVD located at the C-terminal part of the fusion protein comprises a C-terminal A (SEQ ID NO.: 18). Hence, in a preferred embodiment, the fusion protein comprises four ISVDs, three of them comprising or consisting of SEQ ID NO.: 4, one comprising or consisting of SEQ ID NO.: 18, and two peptide linkers (preferably as depicted in SEQ ID NO.: 52 or SEQ ID NO.: 45) which link the ISVDs to each other. The ISVD located at the C-terminal part of the fusion protein preferably comprises a C-terminal A (SEQ ID NO.: 18). For instance, in this embodiment, the fusion protein may comprise or consist of SEQ ID NO.: 85 or a protein which comprises or consists of a sequence with at least 90%, such as at least 95%, or at least 97%, or at least 99% sequence identity with SEQ ID NO.: 85.

In another embodiment, the fusion protein of the present technology comprises a polypeptide comprising two ISVDs, one comprising or consisting of SEQ ID NO.: 54, or a sequence which comprises or consists of a sequence with at least 90%, such as at least 95%, or at least 97%, or at least 99% sequence identity with SEQ ID NO.: 54 and another one comprising or consisting of SEQ ID NO.: 20, or a sequence which comprises or consists of a sequence with at least 90%, such as at least 95%, or at least 97%, or at least 99% sequence identity with SEQ ID NO.: 20, and two further moieties, which are two ISVDs, one comprising or consisting of SEQ ID NO.: 4, or a sequence which comprises or consists of a sequence with at least 90%, such as at least 95%, or at least 97%, or at least 99% sequence identity with SEQ ID NO.: 4 and another one comprising or consisting of SEQ ID NO.: 18, or a sequence which comprises or consists of a sequence with at least 90%, such as at least 95%, or at least 97%, or at least 99% sequence identity with SEQ ID NO.: 18. Preferably, the four ISVDs are linked directly or through a peptide linker, preferably through a peptide linker, such as a peptide linker as depicted in Table 4, e.g., a 35GS linker (SEQ ID NO.: 52). In one embodiment, the fusion protein comprises four ISVDs comprising or consisting of SEQ ID NO.: 54, 20, 4 and 18, and two peptide linkers (preferably as depicted in SEQ ID NO.: 52) which link the ISVDs to each other. Preferably, the ISVD located at the C-terminal part of the fusion protein comprises a C-terminal A (SEQ ID NO.: 18).

In a preferred embodiment, the fusion proteins of the present technology are selected from SEQ ID NO.: 82-86, or a sequence which comprises or consists of a sequence with at least 90%, such as at least 95%, or at least 97%, or at least 99% sequence identity with SEQ ID NO.: 82-86.

TABLE 6
Examples of fusion proteins of the present technology (“ID” refers to the SEQ ID NO
as used herein)

| | | | Schematic |
|---|---|---|---|
| | | | drawing (see |
| Description | ID | Amino acid sequence | FIG. 3) |
| ALB23002-35GS- | 82 | EVQLVESGGGVVQPGGSLRLSCAASGFTFRSFGMSWVRQ | |
| ALB23002-35GS- | | APGKGPEWVSSISGSGSDTLYADSVKGRFTISRDNSKNTLYL | |
| ALB23002-A | | QMNSLRPEDTALYYCTIGGSLSRSSQGTLVTVSSGGGGSGG | |
| (ALB23002-35GS- | | GGSGGGGSGGGGSGGGGSGGGGSGGGGSEVQLVESGGG | |
| ALB23002-35GS- | | VVQPGGSLRLSCAASGFTFRSFGMSWVRQAPGKGPEWVS | |
| ALB223) | | SISGSGSDTLYADSVKGRFTISRDNSKNTLYLQMNSLRPEDT | |
| | | ALYYCTIGGSLSRSSQGTLVTVSSGGGGSGGGGSGGGGSG | |
| | | GGGSGGGGSGGGGSGGGGSEVQLVESGGGVVQPGGSLR | |
| | | LSCAASGFTFRSFGMSWVRQAPGKGPEWVSSISGSGSDTL | |
| | | YADSVKGRFTISRDNSKNTLYLQMNSLRPEDTALYYCTIGGS | |
| | | LSRSSQGTLVTVSSA | |
| ALBX00002-35GS- | 83 | EVQLVESGGGVVQPGGSLRLSCAASGLTFSSYAMGWFRQA | |
| TO23500029- | | PGKERERVVSISRGGGYTYYADSVKGRFTISRDNSKNTVYLQ | |
| 35GS-ALB23002-A | | MNSLRPEDTALYYCAAARYWATGSEYEFDYWGQGTLVTVS | |
| (ALBX00002- | | SGGGGSGGGGSGGGGSGGGGSGGGGSGGGGSGGGGSE | |
| 35GS- | | VQLVESGGGVVQPGDSLRLSCAASGGTFSTYVMGWFRQA | |
| T023500029- | | PGKEREFVSAISQNSIHTYYANSVKGRFTISRDNSKNTVYLQL | |
| 35GS-ALB223) | | NSLRPEDTALYYCAASRFTSWYTADYEYDYWGQGTLVTVSS | |
| | | GGGGSGGGGSGGGGSGGGGSGGGGSGGGGSGGGGSEV | |
| | | QLVESGGGVVQPGGSLRLSCAASGFTFRSFGMSWVRQAP | |
| | | GKGPEWVSSISGSGSDTLYADSVKGRFTISRDNSKNTLYLQ | |
| | | MNSLRPEDTALYYCTIGGSLSRSSQGTLVTVSSA | |
| DARPIN-35GS- | 84 | DLGKKLLEAARAGQDDEVRELLKAGADVNAKDYFSHTPLHL | |
| ALB23002-35GS- | | AARNGHLKIVEVLLKAGADVNAKDFAGKTPLHLAANEGHLE | |
| ALB23002-A | | IVEVLLKAGADVNAQDIFGKTPADIAADAGHEDIAEVLQKA | |
| (DARPIN-35GS- | | AGGGGSGGGGSGGGGSGGGGSGGGGSGGGGSGGGGSE | |
| ALB23002-35GS- | | VQLVESGGGVVQPGGSLRLSCAASGFTFRSFGMSWVRQA | |
| ALB223) | | PGKGPEWVSSISGSGSDTLYADSVKGRFTISRDNSKNTLYLQ | |
| | | MNSLRPEDTALYYCTIGGSLSRSSQGTLVTVSSGGGGSGGG | |
| | | GSGGGGSGGGGSGGGGSGGGGSGGGGSEVQLVESGGGV | |
| | | VQPGGSLRLSCAASGFTFRSFGMSWVRQAPGKGPEWVSSI | |
| | | SGSGSDTLYADSVKGRFTISRDNSKNTLYLQMNSLRPEDTAL | |
| | | YYCTIGGSLSRSSQGTLVTVSSA | |
| ALB23002-35GS- | 85 | EVQLVESGGGVVQPGGSLRLSCAASGFTFRSFGMSWVRQ | |
| ALB23002-35GS- | | APGKGPEWVSSISGSGSDTLYADSVKGRFTISRDNSKNTLYL | |
| ALB23002-35GS- | | QMNSLRPEDTALYYCTIGGSLSRSSQGTLVTVSSGGGGSGG | |
| ALB23002-A | | GGSGGGGSGGGGSGGGGSGGGGSGGGGSEVQLVESGGG | |
| (ALB23002-35GS- | | VVQPGGSLRLSCAASGFTFRSFGMSWVRQAPGKGPEWVS | |
| ALB23002-35GS- | | SISGSGSDTLYADSVKGRFTISRDNSKNTLYLQMNSLRPEDT | |
| ALB23002-35GS- | | ALYYCTIGGSLSRSSQGTLVTVSSGGGGSGGGGSGGGGSG | |
| ALB223) | | GGGSGGGGSGGGGSGGGGSEVQLVESGGGVVQPGGSLR | |
| | | LSCAASGFTFRSFGMSWVRQAPGKGPEWVSSISGSGSDTL | |
| | | YADSVKGRFTISRDNSKNTLYLQMNSLRPEDTALYYCTIGGS | |
| | | LSRSSQGTLVTVSSGGGGSGGGGSGGGGSGGGGSGGGGS | |
| | | GGGGSGGGGSEVQLVESGGGVVQPGGSLRLSCAASGFTFR | |
| | | SFGMSWVRQAPGKGPEWVSSISGSGSDTLYADSVKGRFTI | |
| | | SRDNSKNTLYLQMNSLRPEDTALYYCTIGGSLSRSSQGTLVT | |
| | | VSSA | |
| ALBX00002-35GS- | 86 | EVQLVESGGGVVQPGGSLRLSCAASGLTFSSYAMGWFRQA | |
| T023500029- | | PGKERERVVSISRGGGYTYYADSVKGRFTISRDNSKNTVYLQ | |
| 35GS-ALB23002- | | MNSLRPEDTALYYCAAARYWATGSEYEFDYWGQGTLVTVS | |
| 35GS-ALB23002-A | | SGGGGSGGGGSGGGGSGGGGSGGGGSGGGGSGGGGSE | |
| (ALBX00002- | | VQLVESGGGVVQPGDSLRLSCAASGGTFSTYVMGWFRQA | |
| 35GS- | | PGKEREFVSAISQNSIHTYYANSVKGRFTISRDNSKNTVYLQL | |
| T023500029- | | NSLRPEDTALYYCAASRFTSWYTADYEYDYWGQGTLVTVSS | |
| 35GS-ALB23002- | | GGGGSGGGGSGGGGSGGGGSGGGGSGGGGSGGGGSEV | |
| 35GS-ALB223) | | QLVESGGGVVQPGGSLRLSCAASGFTFRSFGMSWVRQAP | |
| | | GKGPEWVSSISGSGSDTLYADSVKGRFTISRDNSKNTLYLQ | |
| | | MNSLRPEDTALYYCTIGGSLSRSSQGTLVTVSSGGGGSGGG | |
| | | GSGGGGSGGGGSGGGGSGGGGSGGGGSEVQLVESGGGV | |
| | | VQPGGSLRLSCAASGFTFRSFGMSWVRQAPGKGPEWVSSI | |
| | | SGSGSDTLYADSVKGRFTISRDNSKNTLYLQMNSLRPEDTAL | |
| | | YYCTIGGSLSRSSQGTLVTVSSA | |

In some embodiments, the fusion protein of the present technology comprises or consists of:
Two target building blocks, which are preferably two target ISVDs, directly linked to each other or linked through a linker, preferably linked through a linker selected from Table 4, such as a 95G linker, see, e.g., SEQ ID NO.: 45;
Two albumin binding ISVDs, preferably wherein each ISVD comprises or consists of SEQ ID NO.: 4 or a sequence which comprises or consists of a sequence with at least 90%, such as at least 95%, or at least 97%, or at least 99% sequence identity with SEQ ID NO.: 4, wherein, if one of the albumin binding ISVDs is located at the C-terminal end of the fusion protein, the C-terminal ISVD comprises a C-terminal alanine (i.e., SEQ ID NO.: 18 or a sequence which comprises or consists of a sequence with at least 90%, such as at least 95%, or at least 97%, or at least 99% sequence identity with SEQ ID NO.: 18), wherein both albumin binding ISVDs are directly linked to each other or linked through a linker, preferably linked through a linker selected from Table 4, such as a 9SG linker, see, e.g., SEQ ID NO.: 45, and wherein one of the target building blocks is linked to one of the albumin binding ISVDs directly or through a linker, preferably linked through a linker selected from Table 4, such as a 9SG linker, see, e.g., SEQ ID NO.: 45.

In other embodiments, the fusion protein of the present technology comprises or consists of:
Two target building blocks, which are preferably two target ISVDs, directly linked to each other or linked through a linker, preferably linked through a linker selected from Table 4, such as a 9SG linker, see, e.g., SEQ ID NO.: 45;
Two albumin binding ISVDs, preferably wherein each ISVD comprises or consists of SEQ ID NO.: 4 or a sequence which comprises or consists of a sequence with at least 90%, such as at least 95%, or at least 97%, or at least 99% sequence identity with SEQ ID NO.: 4, wherein, if one of the albumin binding ISVDs is located at the C-terminal end of the fusion protein, the C-terminal ISVD comprises a C-terminal alanine (i.e., SEQ ID NO.: 18 or a sequence which comprises or consists of a sequence with at least 90%, such as at least 95%, or at least 97%, or at least 99% sequence identity with SEQ ID NO.: 18), wherein both albumin binding ISVDs are directly linked to each other or linked through a linker, preferably linked through a linker selected from Table 4, such as a 355G linker, see, e.g., SEQ ID NO.: 52, and wherein one of the target building blocks is linked to one of the albumin binding ISVDs directly or through a linker, preferably linked through a linker selected from Table 4, such as a 9SG linker, see, e.g., SEQ ID NO.: 45.

In other embodiments, the fusion protein of the present technology comprises or consists of:
Two target building blocks, which are preferably two target ISVDs, directly linked to each other or linked through a linker, preferably linked through a linker selected from Table 4, such as a 95G linker, see, e.g., SEQ ID NO.: 45;
Two albumin binding ISVDs, preferably wherein each ISVD comprises or consists of SEQ ID NO.: 4 or a sequence which comprises or consists of a sequence with at least 90%, such as at least 95%, or at least 97%, or at least 99% sequence identity with SEQ ID NO.: 4, wherein, if one of the albumin binding ISVDs is located at the C-terminal end of the fusion protein, the C-terminal ISVD comprises a C-terminal alanine (i.e., SEQ ID NO.: 18 or a sequence which comprises or consists of a sequence with at least 90%, such as at least 95%, or at least 97%, or at least 99% sequence identity with SEQ ID NO.: 18), wherein both albumin binding ISVDs are directly linked to each other or linked through a linker, preferably linked through a linker selected from Table 4, such as a 20SG linker, see, e.g., SEQ ID NO.: 49, and wherein one of the target building blocks is linked to one of the albumin binding ISVDs directly or through a linker, preferably linked through a linker selected from Table 4, such as a 9SG linker, see, e.g., SEQ ID NO.: 45.

In other embodiments, the fusion protein of the present technology comprises or consists of:
Three target building blocks, which are preferably three target ISVDs, directly linked to each other or linked through a linker, preferably linked through a linker selected from Table 4, such as a 95G linker, see, e.g., SEQ ID NO.: 45;
One albumin binding ISVD, preferably wherein the ISVD comprises or consists of SEQ ID NO.: 4 or a sequence which comprises or consists of a sequence with at least 90%, such as at least 95%, or at least 97%, or at least 99% sequence identity with SEQ ID NO.: 4, wherein, if the albumin binding ISVD is located at the C-terminal end of the fusion protein, the C-terminal ISVD comprises a C-terminal alanine (i.e., SEQ ID NO.: 18 or a sequence which comprises or consists of a sequence with at least 90%, such as at least 95%, or at least 97%, or at least 99% sequence identity with SEQ ID NO.: 18), and wherein one of the target building blocks is linked to the albumin binding ISVD directly or through a linker, preferably linked through a linker selected from Table 4, such as a 9SG linker, see, e.g., SEQ ID NO.: 45.

In one embodiment, the fusion protein of the present technology comprises or consists of a sequence selected from SEQ ID NO.: 82-86, or a sequence which comprises or consists of a sequence with at least 90%, such as at least 95%, or at least 97%, or at least 99% sequence identity with a sequence selected from SEQ ID NO.: 82-86.

Preferred fusion proteins and polypeptides of the present technology comprise or consist of SEQ ID NO: 76, 82, 85 or 150, or fusion proteins or polypeptides which have at least 90%, such as at least 95%, or at least 99% identity with the amino acid sequence of any one of SEQ ID NO: 76, 8, 85 or 150.

The further groups, residues, moieties or binding units comprised in the fusion protein of the present technology may for example be chemical groups, residues, moieties, which may or may not by themselves be biologically and/or pharmacologically active. For example, and without limitation, such groups may be linked to the polypeptide of the technology, as further described herein. A fusion protein of the technology may also include additional groups with certain functionalities, such as a label, a toxin, one or more linkers, a binding sequence, etc. These additional functionalities include both amino acid-based and non-amino acid-based groups.

The fusion protein of the present technology may additionally comprise (besides the polypeptide of the present technology) one or more targeting moieties. A “targeting moiety”, as defined herein, is any group, residue, moiety, or binding unit which is capable of being directed through its binding to a target.

Further, the fusion protein of the present technology may additionally comprise one or more therapeutic moieties. A “therapeutic moiety”, as defined herein, is any group, residue, moiety, or binding unit which is capable of exerting a therapeutic activity in the animal and/or human body. The therapeutic moiety may also be in the form of a precursor, which then gets activated to exert its therapeutic activity.

Generally, proteins or polypeptides that comprise a single albumin binding unit will be referred to herein as “monovalent polypeptide”, “monovalent fusion proteins” or “monovalent constructs”.

Polypeptides that comprise of two or more binding units (such as the polypeptide or fusion protein of the present technology) will be referred to herein as “multivalent polypeptide”, “multivalent fusion proteins” or “multivalent constructs”. A polypeptide or fusion protein of the present application that comprises two albumin-binding moieties will be referred to herein as “bivalent polypeptide”, “bivalent protein” or “bivalent construct”. In another embodiment, the polypeptide or fusion protein is at least “trivalent”, i.e., it comprises or consists of at least three binding moieties such as three binding moieties. In another embodiment, the polypeptide or fusion protein is at least “tetravalent”, i.e., it comprises or consists of at least four binding moieties such as four moieties. The polypeptide or fusion protein of the present technology can thus be “trivalent”, “tetravalent”, “pentavalent”, “hexavalent”, “heptavalent”, “octavalent”, “nonavalent”, etc., i.e., the polypeptide or fusion protein comprises or consists of three, four, five, six, seven, eight, nine, etc., binning moieties, respectively.

As also described herein, multivalent polypeptides or proteins of the technology may for example, without limitation, be multispecific (such as bispecific or trispecific) or multiparatopic (such as biparatopic) constructs (or be both multiparatopic and multispecific), and may for example be polypeptides or proteins that comprise at least two binding domains or binding units that are each directed towards a different epitope on the same subunit, polypeptides or proteins that comprise at least two binding domains or binding units that each have a different biological function (for example one binding domain that can block or inhibit receptor-ligand interaction, and one binding domain that does not block or inhibit receptor-ligand interaction), or polypeptides or proteins that comprise at least two binding domains or binding units that are each directed towards a different target.

The term “multispecific” refers to binding to multiple different target molecules. The multivalent polypeptides or proteins can thus be “bispecific”, “trispecific”, “tetraspecific”, etc., i.e., it can bind to two, three, four, etc., different target molecules, respectively. For a general description of multivalent and multispecific polypeptides containing one or more ISVDs and their preparation, reference is also made to Conrath et al., J. Biol. Chem., Vol. 276, 10. 7346-7350, 2001; Muyldermans, Reviews in Molecular Biotechnology 74 (2001), 277-302; as well as to for example WO 96/34103, WO 99/23221, WO 04/041862, WO 2006/122786, WO 2008/020079, WO 2008/142164 or WO 2009/068627.

It will be appreciated that the order of the moieties in the polypeptides and/or fusion proteins of the technology can be chosen according to the needs of the person skilled in the art, as well as the relative affinities which may depend on the location of these binding domains in the polypeptide. Whether the polypeptide or fusion protein comprises one or more linkers to interconnect the binding domains and optionally further groups, residues or moieties is a matter of design choice, as described in detail in the present application. However, some orientations, with or without linkers, may provide preferred binding characteristics in comparison to other orientations. All different possible orientations are encompassed by the technology.

When the polypeptide or fusion protein of the technology has an ISVD at its C-terminal end, then said C-terminal ISVD (and thus, by extension, the entire compound of the technology) preferably has a C-terminal extension at its C-terminal end. This C-terminal extension will be directly linked to the last C-terminal amino acid residue of the C-terminal ISVD, which will usually be the amino acid residue at position 113 according to Kabat (unless the ISVD contains one or more amino acid deletions such that the sequence of the ISVD ends before position 113). Thus, generally, the C-terminal extension will be directly linked to the C-terminal VTVSS sequence (SEQ ID NO: 94) of the C-terminal ISVD or the C-terminal sequence of the C-terminal ISVD that corresponds to the C-terminal ISVD sequence (for example, where said C-terminal sequence of the C-terminal ISVD contains one or more substitutions or deletions compared to the usual VTVSS sequence, such as T110K, T1100, S112K or S112K).

Generally, any C-terminal extension that is used herein (i.e., at the C-terminal end of the polypeptide or fusion protein of the technology) can generally be as described in WO 2012/174741 or WO 2015/173325 (reference is also made to for example WO 2103/024059 and WO 2016/118733). In particular, a C-terminal extension may have the formula (X)ₙ, in which n is 1 to 10, preferably 1 to 5, such as 1, 2, 3, 4 or 5 (and preferably 1 or 2, such as 1); and each X is an (preferably naturally occurring) amino acid residue that is independently chosen from naturally occurring amino acid residues (although according to preferred one aspect, it does not comprise any cysteine residues), and preferably independently chosen from the group consisting of alanine (A), glycine (G), valine (V), leucine (L) or isoleucine (1).

According to some preferred, but non-limiting aspects of such C-terminal extensions X(n), X and n can be as follows:
a) n=1 and X=Ala;
b) n=2 and each X=Ala;
c) n=3 and each X=Ala;
d) n=2 and at least one X=Ala (with the remaining amino acid residue(s) X being independently chosen from any naturally occurring amino acid but preferably being independently chosen from Val, Leu and/or lie);
e) n=3 and at least one X=Ala (with the remaining amino acid residue(s) X being independently chosen from any naturally occurring amino acid but preferably being independently chosen from Val, Leu and/or lie);
f) n=3 and at least two X=Ala (with the remaining amino acid residue(s) X being independently chosen from any naturally occurring amino acid but preferably being independently chosen from Val, Leu and/or lie);
g) n=1 and X=Gly;
h) n=2 and each X=Gly;
i) n=3 and each X=Gly;
j) n=2 and at least one X=Gly (with the remaining amino acid residue(s) X being independently chosen from any naturally occurring amino acid but preferably being independently chosen from Val, Leu and/or lie);
k) n=3 and at least one X=Gly (with the remaining amino acid residue(s) X being independently chosen from any naturally occurring amino acid but preferably being independently chosen from Val, Leu and/or lie);
l) n=3 and at least two X=Gly (with the remaining amino acid residue(s) X being independently chosen from any naturally occurring amino acid but preferably being independently chosen from Val, Leu and/or lie);
m) n=2 and each X=Ala or Gly;
n) n=3 and each X=Ala or Gly;
o) n=3 and at least one X=Ala or Gly (with the remaining amino acid residue(s) X being independently chosen from any naturally occurring amino acid but preferably being independently chosen from Val, Leu and/or lie); or
p) n=3 and at least two X=Ala or Gly (with the remaining amino acid residue(s) X being independently chosen from any naturally occurring amino acid but preferably being independently chosen from Val, Leu and/or lie);
   with aspects (a), (b), (c), (g), (h), (i), (m) and (n) being particularly preferred, with aspects in which n=1 or 2 being preferred and aspects in which n=1 being particularly preferred.

It should also be noted that, preferably, any C-terminal extension present in the polypeptide and/or fusion protein of the technology does not contain a (free) cysteine residue (unless said cysteine residue is used or intended for further functionalization, for example for pegylation).

Some specific, but non-limiting examples of useful C-terminal extensions are the following amino acid sequences: A, AA, AAA, G, GG, GGG, AG, GA, AAG, AGG, AGA, GGA, GAA or GAG, preferably A. Preferably also, when the polypeptide and/or fusion protein of the technology has an ISVD at its C-terminal end, then (at least) said C-terminal ISVD preferably contains, even more preferably in addition to a C-terminal extension as described herein, one or more mutations that reduce binding by pre-existing antibodies (i.e., as described herein for the serum albumin binders of the technology and as more generally described in WO 2012/175741 and WO 2015/173325 and also for example in WO 2013/024059 and WO 2016/118733).

More generally, according to a specific aspect of the technology, when the polypeptide and/or fusion protein of the technology contains two or more ISVDs, then preferably all these ISVDs contain mutations that reduce binding to pre-existing antibodies (again, preferably in addition to the C-terminal extension that is linked to the C-terminal ISVD if the polypeptide and/or fusion protein of the technology has an ISVD at its C-terminal end).

When the polypeptide and/or fusion protein of the technology has an ISVD at its N-terminal end, then said N-terminal ISVD (and thus, by extension, the entire polypeptide and/or fusion protein of the technology) preferably contains a D at position 1

### Specificity

Specific binding of a binding unit (such as an ISVD, a HSA, a DARPin, an Affitin or an ABD, as described herein), of the polypeptide and/or of the fusion protein of the present technology to its designated target can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein. The dissociation constant may be the actual or apparent dissociation constant, as will be clear to the skilled person. Methods for determining the dissociation constant will be clear to the skilled person, and for example include the techniques mentioned below. In this respect, it will also be clear that it may not be possible to measure dissociation constants of more than 10⁻⁴ moles/liter or 10⁻³ moles/liter (e.g., of 10⁻² moles/liter). Optionally, as will also be clear to the skilled person, the (actual or apparent) dissociation constant may be calculated on the basis of the (actual or apparent) association constant (K_{A}), by means of the relationship [K_{D}=1/K_{A}]. The affinity of a molecular interaction between two molecules can be measured via different techniques known per se, such as the well-known surface plasmon resonance (SPR) biosensor technique (see for example Ober et al., 2001, Intern. Immunology 13: 30 1551-1559). The term “surface plasmon resonance” (SPR), as used herein, refers to an optical phenomenon that allows for the analysis of real-time biospecific interactions by detection of alterations in protein concentrations within a biosensor matrix, where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known BIAcore® system (BIAcore International AB, a GE Healthcare company, Uppsala, Sweden and Piscataway, NJ) or the ProteOn™ (Bio-Rad Laboratories, Inc) system. For further descriptions, see Jonsson et al. (1993, Ann. Biol. Clin. 51: 19-26), Jonsson et al. (1991 Biotechniques 11: 620-627), Johnsson et al. (1995, J. Mol. Recognit. 8: 125-131), and Johnson et al. (1991, Anal. Biochem. 198: 268-277). Another well-known biosensor technique to determine affinities of biomolecular interactions is bio-layer interferometry (BLI) (see for example Abdiche et al., 2008, Anal. Biochem. 377: 209-217). The term “bio-layer Interferometry” or “BLI”, as used herein, refers to a label-free optical technique that analyzes the interference pattern of light reflected from two surfaces: an internal reference layer (reference beam) and a layer of immobilized protein on the biosensor tip (signal beam). A change in the number of molecules bound to the tip of the biosensor causes a shift in the interference pattern, reported as a wavelength shift (nm), the magnitude of which is a direct measure of the number of molecules bound to the biosensor tip surface. Since the interactions can be measured in real-time, association and dissociation rates and affinities can be determined. BLI can for example be performed using the well-known Octet® Systems (ForteBio, a division of Pall Life Sciences, Menlo Park, USA). Alternatively, affinities can be measured in Kinetic Exclusion Assay (KinExA) (see for example Drake et al. 2004, Anal. Biochem., 328: 35-43), using the KinExA® platform (Sapidyne Instruments Inc, Boise, USA). The term “KinExA”, as used herein, refers to a solution-based method to measure true equilibrium binding affinity and kinetics of unmodified molecules. Equilibrated solutions of an antibody/antigen complex are passed over a column with beads precoated with antigen (or antibody), allowing the free antibody (or antigen) to bind to the coated molecule. Detection of the antibody (or antigen) thus captured is accomplished with a fluorescently labelled protein binding the antibody (or antigen). The GYROLAB® immunoassay system provides a platform for automated bioanalysis and rapid sample turnaround (Fraley et al., 2013, Bioanalysis 5: 1765-74).

Typically, binding units (such as ISVDs, HSA, DARPins, Affitins or ABDs, as described herein), or polypeptides or fusion proteins of the present technology will bind to their targets with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e., with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁻⁴ mol/liter (or any K_{A} value lower than 10⁴ liters/mol) is generally considered to indicate non-specific binding. The K_{D} for biological interactions, such as the binding of immunoglobulin sequences to an antigen, which are considered specific are typically in the range of 10⁻⁵ moles/liter (10000 nM or 10 μM) to 10⁻¹² moles/liter (0.001 nM or 1 pM) or less. Accordingly, specific/selective binding may mean that—using the same measurement method, e.g., SPR—a binding unit (or polypeptide comprising the same) binds to their targets with a K_{D} value of 10⁻⁵ to 10⁻¹² moles/liter or less and binds to related targets with a K_{D} value greater than 10⁻⁴ moles/liter.

In particular embodiments, the binding units (such as ISVDs, HSA, DARPins, Affitins or ABDs, as described herein), or polypeptides or fusion proteins of the present technology comprise at least one domain that specifically binds to a serum albumin protein, such as to HSA, with an affinity (K_{A}) of between 10⁵ M⁻¹ and 10¹¹ M⁻¹, such as between 10⁶ M⁻¹ and 10¹¹ M⁻¹.

In particular embodiments, the polypeptides and/or fusion proteins comprise at least one domain specifically binding to a serum albumin protein (such as an ISVDs, a HSA, a DARPin, an Affitin or an ABD, as described herein) with a dissociation constant (K_{D}) of between 10⁻⁶ M and 8.5*10⁻¹¹ M or less, such as of between 10⁻⁶ M and 10⁻¹¹ M or less. Preferably, the K_{D} is determined by Kinexa, BLI or SPR, for instance as determined by SPR.

In particular embodiments, the binding units (such as ISVDs, HSA, DARPins, Affitins or ABDs, as described herein), or polypeptides or fusion proteins of the present technology comprise are generally preferably such that they bind to human serum albumin with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹, as determined using ProteOn. Preferably, a serum albumin binder of the technology (such as ISVDs, HSA, DARPins, Affitins or ABDs, as described herein) will bind to the desired antigen with an affinity less than 500 nM, preferably less than 200 nM, more preferably less than 10 nM, such as less than 500 pM, as determined using ProteOn.

In particular embodiments, the polypeptides and/or fusion proteins of the technology comprise at least one domain specifically binding to a serum albumin protein (such as ISVDs, HSA, DARPins, Affitins or ABDs, as described herein) with an on-rate constant (kₒₙ) selected from the group consisting of at least about 10² M⁻¹s⁻¹, of at least about 10³ M⁻¹s⁻¹, at least about 10⁴ M⁻¹s⁻¹, at least about 10⁵ M⁻¹s⁻¹, at least about 10⁶M⁻¹s⁻¹, at least about 10⁷ M⁻¹s⁻¹, and at least about 10⁸M⁻¹s⁻¹, preferably as measured by surface plasmon resonance or BLI.

In particular embodiments, the polypeptides and/or fusion proteins of the present technology comprise at least one domain specifically binding to a serum albumin protein (such as ISVDs, HSA, DARPins, Affitins or ABDs, as described herein) with an off-rate constant (k_{off}) selected from the group consisting of at most about 10⁻¹ s⁻¹, at most about 10⁻² s⁻¹, at most about 10⁻³ s⁻¹, of at most about 10⁻⁴s⁻¹, at most about 10⁻⁵s⁻¹, and at most about 10⁻⁶s⁻¹, preferably as measured by surface plasmon resonance or BLI.

The binding units (such as ISVDs, HSA, DARPins, Affitins or ABDs, as described herein), polypeptides and/or fusion proteins according to the different aspects of the technology are generally preferably also such that they are cross-reactive between human serum albumin and serum albumin from at least one, preferably from at least two, more preferably from at least three and up to essentially all of the following species of mammal: rat, mouse, rabbit, guinea pig, pig, sheep, cow and cynomolgus monkey. In particular, the serum albumin binders (binding units such as ISVDs, HSA, DARPins, Affitins or ABDs, as described herein, polypeptides and/or fusion proteins) according to the different aspects of the present technology may be such that they are (at least) cross-reactive between human serum albumin and at least one, preferably at least two, more preferably at all three of rat serum albumin, mouse serum albumin and serum albumin from cynomolgus monkey. In this respect, the serum albumin binders of the technology may have improved cross-reactivity (in particular between human serum albumin on the one hand and rat and/or mouse serum albumin on the other hand) compared to serum albumin binders that have (essentially) the same CDR's (according to AbM numbering) as Alb-11 and/or Alb23002(E1D), see, e.g., SEQ ID NO.: 9 (Alb-11) or SEQ ID NO.: 19 (Alb23002(E1D)).

When a binding unit (such as ISVDs, HSA, DARPins, Affitins or ABDs, as described herein) or a polypeptide, or a fusion protein of the present technology is said to exhibit “(improved) cross-reactivity for binding to human and non-human primate serum albumin” compared to another binding unit (such as ISVDs, HSA, DARPins, Affitins or ABDs, as described herein) or polypeptide, or fusion protein, it means that for said binding unit (such as ISVDs, HSA, DARPins, Affitins or ABDs, as described herein) or polypeptide, or fusion protein, the ratio of the binding activity (such as expressed in terms of K_{D} or k_{off}) for human serum albumin and for non-human primate serum albumin is lower than that same ratio calculated for the other binding unit (such as ISVDs, HSA, DARPins, Affitins or ABDs, as described herein) or polypeptide, or fusion protein in the same assay. Good cross-reactivity for binding to human and non-human primate serum albumin allows for the assessment of toxicity of a serum albumin binding polypeptide according to the present technology in preclinical studies conducted on non-human primates.

In particular embodiments, the at least one serum albumin binding domain comprised in the polypeptides of the technology is such that it is (at least) cross-reactive between human serum albumin and cynomolgus monkey serum albumin, and preferably also between either human serum albumin and/or cynomolgus monkey serum albumin on the one hand, and at least one, preferably both of rat serum albumin and pig serum albumin on the other hand. For the sake of convenience, in the sequence of serum albumin, the stretches of amino acids that are assumed to be part of the putative epitope of the polypeptides of the technology have been highlighted. Without being limited to any specific mechanism or hypothesis, it is assumed that the polypeptides and fusion proteins of the technology are (essentially) capable of binding to (one or more amino acid residues within) the corresponding stretches of amino acid residues that are present within the amino acid sequence of those mammalian serum albumin proteins, with which the polypeptides and fusion proteins of the technology are cross-reactive.

Generally, a polypeptide of the technology comprising at least one serum albumin binding moiety can be considered to be cross-reactive between human serum albumin and serum albumin from one of the above mentioned other species when it can bind to human serum albumin with an affinity less than 500 nM, preferably less than 200 nM, more preferably less than 10 nM; and also to serum albumin from those above-mentioned species with an affinity less than 500 nM, preferably less than 200 nM, more preferably less than 10 nM, again both as determined using SPR.

Preferably, in instances where the polypeptide and/or fusion protein of the present technology comprises or consists of two or more target building blocks specifically binding to a serum albumin protein, it is preferable that each of the building blocks binds to a distinct (different) albumin molecule at least at physiological albumin concentrations. Hence, at least at physiological albumin concentrations, it is preferred that the at least two albumin-binding building blocks comprised in the polypeptide or fusion protein of the present technology (and preferably all) bind to at least two albumin molecules (i.e., each albumin-binding building block binds a different albumin molecule). Therefore, in instances where the polypeptide and/or fusion protein of the present technology comprises or consists of two or more albumin-binding building blocks it is preferable that the two or more albumin-binding building blocks of the polypeptide and/or fusion protein of the present technology do not bind to the same serum albumin molecule. For example, in a polypeptide and/or fusion protein of the present technology comprising or consisting of two albumin-binding building blocks, it is preferable that the polypeptide and/or fusion protein of the present technology binds two serum albumin molecules simultaneously. For example, in a polypeptide and/or fusion protein of the present technology comprising or consisting of three albumin-binding building blocks, it is preferable that the polypeptide and/or fusion protein of the present technology binds three serum albumin molecules simultaneously.

In embodiments where the polypeptide and/or fusion protein of the present technology comprises or consists of (i) at least one ISVD which specifically binds to a serum albumin protein; and (ii) at least one further moiety specifically binding to a serum albumin protein, each of (i) and (ii) may not bind the same or overlapping epitopes on serum albumin. In other embodiments, if the polypeptide and/or fusion protein of the present technology comprises or consists of (i) at least one ISVD which specifically binds to a serum albumin protein; and (ii) at least one further moiety specifically binding to a serum albumin protein, each of (i) and (ii) (and each of the other albumin-binding building blocks, if present) bind the same or overlapping epitopes on serum albumin, but each of the albumin-binding building blocks comprised in the polypeptide and/or fusion protein bind a different albumin molecule, at least at physiological albumin concentrations.

### Half-Life

The term “half-life” as used here can generally be defined as described in paragraph o) on page 57 of WO 2008/020079 and as mentioned therein refers to the time taken for the serum concentration of the compound or polypeptide to be reduced by 50%, in vivo, for example due to degradation of the sequence or compound and/or clearance or sequestration of the sequence or compound by natural mechanisms. The in vivo half-life of the polypeptide and/or fusion protein of the technology can be determined in any manner known per se, such as by pharmacokinetic analysis. Suitable techniques will be clear to the person skilled in the art and may for example generally be as described in paragraph o) on page 57 of WO 2008/020079. As also mentioned in paragraph o) on page 57 of WO 2008/020079, the half-life can be expressed using parameters such as the t_{1/2}-alpha, t_{1/2}-beta and the area under the curve (AUC). In this respect it should be noted that the term “half-life” as used herein in particular refers to the t_{1/2}-beta or terminal half-life (in which the tin-alpha and/or the AUC or both may be kept out of considerations). Reference is for example made to the standard handbooks, such as Kenneth, A et al: Chemical Stability of Pharmaceuticals: A Handbook for Pharmacists and Peters et al, Pharmacokinetic analysis: A Practical Approach (1996). Reference is also made to “Pharmacokinetics”, M Gibaldi & D Perron, published by Marcel Dekker, 2nd Rev. edition (1982). Similarly, the terms “increase in half-life” or “increased half-life” are also as defined in paragraph o) on page 57 of WO 2008/020079 and in particular refer to an increase in the tin-beta, either with or without an increase in the t_{1/2}-alpha and/or the AUC or both.

The half-life in mammalian species (e.g., mice) will, among other factors, mainly depend on the binding properties (such as affinity) of the polypeptides and/or fusion proteins of the technology for the serum albumin from said mammalian species as well on the half-life of the naïve serum albumin in said species. According to a preferred embodiment of the technology, when a polypeptide and/or fusion protein of the technology is cross-reactive (as defined herein) between human serum albumin and serum albumin from another mammalian species (e.g., mice), then the half-life of the polypeptide and/or fusion protein of the technology as determined in said species is preferably at least 5%, such as at least 10%, more preferably at least 25%, for example about 50% and possibly up to 500%, such as 100%, 150% or 200% of the half-life of the naïve serum albumin in said species. Preferably, the half-life of the polypeptide and/or fusion protein of the technology as determined in said species is preferably at least 100%, such as at least 150%, more preferably at least 200%, or more, of the half-life of the naïve serum albumin in said species. Hence, the half-life of the polypeptide and/or fusion protein of the technology as determined in said species is preferably at least 1.1 times, at least 1.2 times, at least 1.3 times, at least 1.4 times, at least 1.5 times, at least 1.7 times, or at least 1.8 times, preferably at least 2 times, preferably at least 2.3 times, or at least 2.4 times, or at least 2.5 times, or at least 3 times, at least 4 times, such as at least 5 times, for example at least 10 times or more than 20 times, greater than the half-life of the naïve serum albumin in said species. In particular, the polypeptide and/or fusion protein has a serum half-life in man that is at least 5%, such as at least 10%, at least 25%, at least 50%, at least 100%, at least 200%, at least 300%, at least 400% or at least 500% of the half-life of serum albumin in humans. Hence, the half-life of the polypeptide and/or fusion protein of the technology as determined in said species is preferably at least 1.1 times, at least 1.2 times, at least 1.3 times, at least 1.4 times, at least 1.5 times, at least 1.7 times, preferably at least 2 times, preferably at least 2.1 times, or at least 2.2 times, or at least 2.3 times, or at least 2.4 times, or at least 3 times, or at least 4 times, such as at least 5 times, for example at least 10 times or more than 20 times, greater than the half-life of the serum albumin in humans (HSA).

Also, the polypeptides according to the technology, if fused to another moiety, such as a therapeutic moiety or moieties, will have an increased half-life, compared to the other moiety per se, such as the other therapeutic moiety or moieties per se.

The polypeptide and/or fusion protein described herein preferably have a half-life that is at least 2 times, such as at least 5 times, preferably at least 10 times or more than 20 times, such as more than 50 times, more than 100 times, more than 500 times, preferably more than 1000 times greater than the half-life of the corresponding other moiety per se, such as for example a therapeutic moiety per se (as measured in either man or a suitable animal, such as mouse or cynomolgus monkey).

Also, the polypeptides and/or fusion proteins of the present technology will have an increased half-life, compared to polypeptides comprising a single albumin binding moiety per se, such as a single albumin binding ISVD, or a single other albumin binding moiety (ABD, Darpin, Affitin) per se. In particular, the polypeptides according to the technology, will have an increased half-life as compared to a construct comprising a therapeutic moiety a single known half-life extending moiety as disclosed in the prior art.

The polypeptides and/or fusion proteins described herein preferably have a half-life that is at least 1.1 times, 1.2 times, 1.3 times, 1.4 times, 1.5 times, preferably at least 2 times, preferably at least 2.4 times, at least 3 times, at least 4 times, such as at least 5 times, for example at least 10 times or more than 20 times, greater than the half-life of a corresponding construct comprising a single known half-life extending moiety as disclosed in the prior art (as measured in either in man or a suitable animal, such as mouse or cynomolgus monkey). In particular, the polypeptides and/or fusion proteins described herein preferably have a half-life that is at least 1.5 times, or at least 1.7, or at least 1.8, preferably at least 2 times, such as at least 2.1 times, at least 2.2 times, at least 2.3 times, at least 2.4 times or at least 2.5 times, greater than the half-life of a corresponding construct comprising a single known half-life extending moiety as disclosed in the prior art (as measured in either in man or a suitable animal, such as mouse or cynomolgus monkey).

The polypeptides and/or fusion proteins described herein preferably have a half-life that is at least 1.1 times, 1.2 times, 1.3 times, 1.4 times, 1.5 times, preferably at least 2 times, preferably at least 2.3 times, or at least 2.4 times, or at least 2.5 times, at least 3 times, at least 4 times, such as at least 5 times, for example at least 10 times or more than 20 times, greater than the half-life of a therapeutic construct comprising a therapeutic moiety and a single known half-life extending moiety as disclosed in the prior art (as measured in either in man or a suitable animal, such as mouse or cynomolgus monkey). In particular, the polypeptides and/or fusion proteins described herein preferably have a half-life that is at least 1.5 times, or at least 1.7, or at least 1.8, preferably at least 2 times, such as at least 2.1 times, at least 2.2 times, at least 2.3 times, or at least 2.4 times, or at least 2.5 times, greater than the half-life of a therapeutic construct comprising a therapeutic moiety and a single known half-life extending moiety as disclosed in the prior art (as measured in either in man or a suitable animal, such as mouse or cynomolgus monkey).

As mentioned, in one aspect, a polypeptide according to the present technology can be used to increase the half-life of (one or more) immunoglobulin single variable domains (ISVDs), such as domain antibodies, single domain antibodies, “dAb's”, V_{HH}s or Nanobody V_{HH}s (such as V_{HH}s, humanized V_{HH}s or camelized VHS such as camelized human V_{H}s).

Methods for Preparing the Polypeptides and/or Fusion Proteins of the Present Technology

Another embodiment of the technology relates to a method for producing the polypeptide and/or fusion protein of the present technology.

As described in detail above, the polypeptide and/or fusion protein according to the present technology comprises (i) at least one immunoglobulin single variable domain (ISVD) which specifically binds to a serum albumin protein or at least one human serum albumin protein and (ii) at least one further moiety specifically binding to a serum albumin protein or at least one human serum albumin protein.

Thus, the polypeptides of the technology can generally be prepared by a method which comprises at least one step of suitably linking the one or more components (i) and (ii), including the linker, if present, e.g., one ISVD, one peptide linker (e.g., 35GS, 9GS or 20GS) and the domain comprising a serum albumin protein or the serum albumin binding domain to each other.

The fusion protein of the present technology can be prepared by a method similar to the one for preparing the polypeptide of the technology.

Polypeptides and fusion proteins of the technology can also be prepared by a method which generally comprises at least the steps of providing a nucleic acid that encodes the polypeptide or fusion protein of the technology, expressing said nucleic acid in a suitable manner, and recovering the expressed polypeptide of the technology. Such methods can be performed in a manner known per se, which will be clear to the skilled person, for example on the basis of the methods and techniques further described herein. The process of designing/selecting and/or preparing a polypeptide or fusion protein of the technology, starting from a polypeptide or fusion protein comprising at least one component (e.g., component (i) or (ii)), is also referred to herein as “formatting” said polypeptide or fusion protein of the technology. Examples of ways in which a polypeptide or fusion protein of the technology can be formatted, and examples of such formats will be clear to the skilled person based on the disclosure herein.

The skilled person is aware of means of linking two polypeptides to prepare the polypeptide and/or fusion protein of the present technology. For instance, the method may comprise the steps of:
a) Providing at least a ISVD or HSA (i) and a further moiety or HSA (ii), as described above;
c) Optionally providing one further group, residue, moiety or binding unit;
b) Linking all polypeptides together, directly or by means of a linker (see, e.g., Table 4), as described herein.

For instance, the method may comprise the steps of:
a) Selecting at least a ISVD or HSA (i) and a further moiety or HSA (ii), as described above and optionally at least one further group, residue, moiety or binding unit;
b) Designing a genetic construct which encodes a protein sequence comprising the polypeptides of a); and
c) Introducing said genetic construct in an expression system to obtain the polypeptide or fusion protein of the present technology, as described above in the present specification.

In the context of the present technology, the position of each of the components ((i) and (ii)) in the polypeptide of the present technology is not limited. For instance, the first component (i) may be located in the N-terminal part of the polypeptide, whereas the at least one further moiety specifically binding to a serum albumin protein or at least one human serum albumin protein (ii) may be located in the C-terminal part of the polypeptide. In addition, the first component (i) may be located in the C-terminal part of the polypeptide, whereas the at least one further moiety specifically binding to a serum albumin protein or at least one human serum albumin protein (ii) may be located in the N-terminal part of the polypeptide. The same is true for the fusion protein of the present technology; the polypeptide of the present technology may be located in the C-terminal part of the fusion protein, whereas the at least one further group, residue, moiety or binding unit may be located in the N-terminal part of the fusion protein, and vice versa (the polypeptide of the present technology may be located in the N-terminal part of the fusion protein, whereas the at least one further group, residue, moiety or binding unit may be located in the C-terminal part of the fusion protein.

### Nucleic Acid Molecules

The present technology also provides a nucleic acid molecule encoding the polypeptides or fusion proteins of the present technology.

A “nucleic acid molecule” (used interchangeably with “nucleic acid”) is a chain of nucleotide monomers linked to each other via a phosphate backbone to form a nucleotide sequence. A nucleic acid may be used to transform/transfect a host cell or host organism, e.g., for expression and/or production of a polypeptide or fusion protein. Suitable (non-human) hosts or host cells for production purposes will be clear to the skilled person, and may for example be any suitable fungal, prokaryotic or eukaryotic cell or cell line or any suitable fungal, prokaryotic or eukaryotic organism. A host or host cell comprising a nucleic acid encoding the protein-based carrier building block and/or the molecule (or part of the molecule) of the present technology is also encompassed by the present technology.

A nucleic acid may be for example DNA, RNA, or a hybrid thereof, and may also comprise (e.g., chemically) modified nucleotides, like PNA. It can be single- or double-stranded. In one embodiment, it is in the form of double-stranded DNA. For example, the nucleotide sequences of the present technology may be genomic DNA, cDNA.

The nucleic acids of the present technology can be prepared or obtained in a manner known per se, and/or can be isolated from a suitable natural source. Nucleotide sequences encoding naturally occurring (poly)peptides can for example be subjected to site-directed mutagenesis, so as to provide a nucleic acid molecule encoding polypeptide with sequence variation. Also, as will be clear to the skilled person, to prepare a nucleic acid, also several nucleotide sequences, such as at least one nucleotide sequence encoding a targeting moiety and for example nucleic acids encoding one or more linkers can be linked together in a suitable manner.

Techniques for generating nucleic acids will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers.

### Vectors

Also provided is a vector comprising the nucleic acid molecule encoding the polypeptides or fusion proteins of the present technology.

A vector as used herein is a vehicle suitable for carrying genetic material into a cell. A vector includes naked nucleic acids, such as plasmids or mRNAs, or nucleic acids embedded into a bigger structure, such as liposomes or viral vectors.

In some embodiments, vectors comprise at least one nucleic acid that is optionally linked to one or more regulatory elements, such as for example one or more suitable promoter(s), enhancer(s), terminator(s), etc.). In one embodiment, the vector is an expression vector, i.e., a vector suitable for expressing an encoded polypeptide or construct under suitable conditions, e.g., when the vector is introduced into a (e.g., human) cell. DNA-based vectors include the presence of elements for transcription (e.g., a promoter and a polyA signal) and translation (e.g., Kozak sequence).

In one embodiment, in the vector, said at least one nucleic acid and said regulatory elements are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor). Generally, when two nucleotide sequences are operably linked, they will be in the same orientation and usually also in the same reading frame. They will usually also be essentially contiguous, although this may also not be required.

In one embodiment, any regulatory elements of the vector are such that they are capable of providing their intended biological function in the intended host cell or host organism.

For instance, a promoter, enhancer or terminator should be “operable” in the intended host cell or host organism, by which is meant that for example said promoter should be capable of initiating or otherwise controlling/regulating the transcription and/or the expression of a nucleotide sequence—e.g., a coding sequence—to which it is operably linked.

### The Host Cell of the Present Technology

The nucleic acids of the technology and/or the genetic constructs of the technology (nucleic acids of the technology) may be used to transform a host cell or host organism, i.e., for expression and/or production of the polypeptides and/or fusion proteins of the technology. The host is preferably a non-human host. Suitable hosts or host cells will be clear to the skilled person, and may for example be any suitable fungal, prokaryotic or eukaryotic cell or cell line or any suitable fungal, prokaryotic or eukaryotic organism, for example: a bacterial strain, including but not limited to gram-negative strains such as strains of Escherichia coli; of Proteus, for example of Proteus mirabilis; of Pseudomonas, for example of Pseudomonas fluorescens; and gram-positive strains such as strains of Bacillus, for example of Bacillus subtilis or of Bacillus brevis; of Streptomyces, for example of Streptomyces lividons; of Staphylococcus, for example of Staphylococcus carnosus; and of Lactococcus, for example of Lactococcus lactis; a fungal cell, including but not limited to cells from species of Trichoderma, for example from Trichoderma reesei; of Neurospora, for example from Neurospora crassa; of Sordaria, for example from Sordaria macrospora; of Aspergillus, for example from Aspergillus niger or from Aspergillus sojae; or from other filamentous fungi; a yeast cell, including but not limited to cells from species of Saccharomyces, for example of Saccharomyces cerevisiae; of Schizosaccharomyces, for example of Schizosaccharomyces pombe; of Pichia, for example of Pichia pastoris or of Pichia methanolica; of Hansenula, for example of Hansenula polymorpha; of Kluyveromyces, for example of Kluyveromyces lactis; of Arxula, for example of Arxula adeninivorans; of Yarrowia, for example of Yarrowia lipolytica; an amphibian cell or cell line, such as Xenopus oocytes; an insect-derived cell or cell line, such as cells/cell lines derived from lepidoptera, including but not limited to Spodoptera SF9 and Sf21 cells or cells/cell lines derived from Drosophila, such as Schneider and Kc cells; a plant or plant cell, for example in tobacco plants; and/or a mammalian cell or cell line, for example a cell or cell line derived from a human, a cell or a cell line from mammals including but not limited to CHO-cells, BHK-cells (for example BHK-21 cells) and human cells or cell lines such as HeLa, COS (for example COS-7) and PER.C6 cells; as well as all other hosts or host cells known per se for the expression and production of antibodies and antibody fragments (including but not limited to (single) domain antibodies and ScFv fragments), which will be clear to the skilled person. Reference is also made to the general background art cited hereinabove, as well as to for example WO 94/29457; WO 96/34103; WO 99/42077; Frenken et al. 1998 (Res. Immunol. 149: 589-99); Riechmann and Muyldermans 1999 (J. Immunol. Met. 231: 25-38); van der Linden 2000 (J. Biotechnol. 80: 261-70); Joosten et al. 2003 (Microb. Cell Fact. 2: 1); Joosten et al. 2005 (Appl. Microbiol. Biotechnol. 66: 384-92); and the further references cited herein.

For expression of the Polypeptides and/or fusion proteins in a cell, they may also be expressed as so-called “intrabodies”, as for example described in WO 94/02610, WO 95/22618 and U.S. Pat. No. 7,004,940; WO 03/014960; in Cattaneo and Biocca 1997 (Intracellular Antibodies: Development and Applications. Landes and Springer-Verlag); and in Kontermann 2004 (Methods 34: 163-170).

According to one preferred, but non-limiting embodiment of the technology, the polypeptides and/or fusion proteins of the technology are produced in a bacterial cell, in particular a bacterial cell suitable for large scale pharmaceutical production, such as cells of the strains mentioned above.

According to another preferred, but non-limiting embodiment of the technology, the polypeptides and/or fusion proteins of the technology are produced in a yeast cell, in particular a yeast cell suitable for large scale pharmaceutical production, such as cells of the species mentioned above.

According to yet another preferred, but non-limiting embodiment of the technology, the polypeptides and/or fusion proteins of the technology are produced in a mammalian cell, in particular in a human cell or in a cell of a human cell line, and more in particular in a human cell or in a cell of a human cell line that is suitable for large scale pharmaceutical production, such as the cell lines mentioned hereinabove.

Suitable techniques for transforming a host or host cell of the technology will be clear to the skilled person and may depend on the intended host cell/host organism and the genetic construct to be used. Reference is again made to the handbooks and patent applications mentioned above.

After transformation, a step for detecting and selecting those host cells or host organisms that have been successfully transformed with the nucleotide sequence/genetic construct of the technology may be performed. This may for instance be a selection step based on a selectable marker present in the genetic construct of the technology or a step involving the detection of the polypeptide of the technology, e.g., using specific antibodies.

The transformed host cell (which may be in the form or a stable cell line) or host organisms (which may be in the form of a stable mutant line or strain) form further aspects of the present technology.

Preferably, these host cells or host organisms are such that they express or are (at least) capable of expressing (e.g., under suitable conditions), the polypeptides/or fusion proteins of the technology (and in case of a host organism: in at least one cell, part, tissue or organ thereof). The technology also includes further generations, progeny and/or offspring of the host cell or host organism of the technology, for instance obtained by cell division or by sexual or asexual reproduction.

Accordingly, in another aspect, the technology relates to a host or host cell that expresses (or that under suitable circumstances is capable of expressing) the polypeptides and/or fusion proteins of the technology; and/or that contains a nucleic acid encoding the same. Some preferred but non-limiting examples of such hosts or host cells can be as generally described in WO 04/041867, WO 04/041865 or WO 09/068627. For example, the polypeptides and/or fusion proteins of the technology may with advantage be expressed, produced or manufactured in mammalian cells, such as Chinese Hamster Overy cells (CHO cells), or in a suitable yeast strain, such as a strain of Pichia pastoris. Reference is also made to WO 04/25591, WO 10/125187, WO 11/003622, and WO 12/056000 which also describes the expression/production in Pichia and other hosts/host cells of immunoglobulin single variable domains and polypeptides comprising the same.

To produce/obtain expression of the polypeptides and/or fusion proteins of the technology, the transformed host cell or transformed host organism may generally be kept, maintained and/or cultured under conditions such that the (desired) polypeptides and/or fusion proteins of the technology are expressed/produced. Suitable conditions will be clear to the skilled person and will usually depend upon the host cell/host organism used, as well as on the regulatory elements that control the expression of the (relevant) nucleotide sequence of the technology. Again, reference is made to the handbooks and patent applications mentioned above in the paragraphs on the genetic constructs of the technology.

Generally, suitable conditions may include the use of a suitable medium, the presence of a suitable source of food and/or suitable nutrients, the use of a suitable temperature, and optionally the presence of a suitable inducing factor or compound (e.g., when the nucleotide sequences of the technology are under the control of an inducible promoter); all of which may be selected by the skilled person. Again, under such conditions, the polypeptides and/or fusion proteins of the technology may be expressed in a constitutive manner, in a transient manner, or only when suitably induced.

It will also be clear to the skilled person that the polypeptides and/or fusion proteins of the technology may (first) be generated in an immature form (as mentioned above), which may then be subjected to post-translational modification, depending on the host cell/host organism used. Also, the polypeptides and/or fusion proteins of the technology may be glycosylated, again depending on the host cell/host organism used.

The polypeptides and/or fusion proteins of the technology may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g., using a specific, cleavable amino acid sequence fused with the polypeptide or construct of the technology) and/or preparative immunological techniques (i.e., using antibodies against the amino acid sequence to be isolated).

An polypeptide or protein is considered to be “(in) essentially isolated (form)”—for example, compared to its native biological source and/or the reaction medium or cultivation medium from which it has been obtained—when it has been separated from at least one other component with which it is usually associated in said source or medium, such as another protein/polypeptide, another biological component or macromolecule or at least one contaminant, impurity or minor component. In particular, a polypeptide or protein is considered “essentially isolated” when it has been purified at least 2-fold, in particular at least 10-fold, more in particular at least 100-fold, and up to 1000-fold or more. A polypeptide or protein that is “in essentially isolated form” is preferably essentially homogeneous, as determined using a suitable technique, such as a suitable chromatographical technique, such as polyacrylamide-gel electrophoresis.

Compositions, Vaccines and Methods of Treatment and/or Prevention

The technology also relates to a composition comprising the polypeptides and/or fusion proteins of the technology. The composition may be a pharmaceutical composition. The composition may further comprise at least one pharmaceutically acceptable carrier, diluent or excipient and/or adjuvant, and optionally comprise one or more further pharmaceutically active polypeptides and/or compounds.

In the above methods, the polypeptides and/or fusion proteins can be administered in any suitable manner, depending on the specific pharmaceutical formulation or composition to be used. Thus, the polypeptides and/or fusion proteins of the technology and/or the compositions comprising the same can for example be administered orally, intraperitoneally, intravenously, subcutaneously, intramuscularly, or via any other route of administration that circumvents the gastrointestinal tract, intranasally, transdermally, topically, by means of a suppository, by inhalation, again depending on the specific pharmaceutical formulation or composition to be used. It may also be administered by mucosal delivery, such as oral delivery or intranasally. The clinician will be able to select a suitable route of administration and a suitable pharmaceutical formulation or composition to be used in such administration, depending on the disease or disorder to be prevented or treated and other factors well known to the clinician.

An effective amount of the polypeptides and/or fusion proteins, or a composition comprising the same can be administered to a subject in order to provide the intended treatment results. As used herein, the term “therapeutic agent” refers to any agent that can be used in the prophylaxis (prevention), treatment and/or management of a disease or disorder or one or more symptoms thereof. In certain embodiments, the term “therapeutic agent” refers to the polypeptides and/or fusion proteins of the technology and/or compositions comprising the same. Preferably, a therapeutic agent is an agent which is known to be useful for, or has been or is currently being used for the treatment, prevention and/or management of a disease or disorder, or one or more symptoms thereof.

As used herein, a “therapeutically effective amount” in the present context refers to the amount of a therapy alone, or in combination with other therapies, that provides a therapeutic benefit in the treatment and/or management of a disease and/or disorder. In one aspect, a therapeutically effective amount refers to the amount of a therapy sufficient to cure, modify, stabilize or control a disease and/or disorder, or one or more symptoms thereof. In another aspect, a therapeutically effective amount refers to the amount of a therapy sufficient to reduce the symptoms of a disease and/or disorder. In another aspect, a therapeutically effective amount refers to the amount of a therapy sufficient to delay or minimize the spread of a disease and/or disorder.

As used herein, the term “therapy” refers to any protocol, method and/or agent that can be used in the treatment, prevention and/or management of a disease and/or disorder, or symptoms thereof. In certain embodiments, the terms “therapies” and “therapy” refer to a biological therapy, supportive therapy, and/or other therapies useful in the treatment, prevention and/or management of a disease and/or disorder, or one or more symptoms thereof known to one of skill in the art, such as medical personnel.

As used herein, the terms “treat”, “treatment” and “treating” in the context of administering (a) therapy(ies) to a subject refer to the reduction or amelioration of the progression, severity, and/or duration of a diseases or disorder, and/or the amelioration of one or more symptoms thereof resulting from the administration of one or more therapies (including, but not limited to, the administration of one or more prophylactic or therapeutic agents). In the context of the present technology, the terms “treat”, “treatment” and “treating” may relate to therapeutic and/or to prophylactic (preventive) treatment. The term “prophylactic treatment” refers to a therapy to reduce the susceptibility to a clinical condition. Thus, the terms “treat”, “treatment”, “treating” and their equivalent terms refer to obtaining a desired pharmacologic or physiologic effect, covering any treatment of a pathological condition, disease or disorder in a mammal, including a human. The effect may be prophylactic in terms of completely or partially preventing pathological condition, disease or disorder or symptom thereof and/or may be therapeutic in terms of a partial or complete cure for a pathological condition, disease or disorder and/or adverse effect attributable to the pathological condition, disease or disorder. That is, “treatment” includes (1) preventing the pathological condition, disease or disorder from occurring or recurring in a subject, (2) inhibiting the pathological condition, disease or disorder, such as arresting its development, (3) stopping or terminating the pathological condition, disease or disorder or, at least, symptoms associated therewith, so that the host no longer suffers from the pathological condition, disease or disorder or its symptoms, such as causing regression of the pathological condition, disease or disorder or its symptoms, or (4) relieving, alleviating, or ameliorating the pathological condition, disease or disorder, or symptoms associated therewith, where ameliorating is used in a broad sense to refer to at least a reduction in the magnitude of a parameter, such as inflammation, pain, or immune deficiency.

The polypeptides or fusion proteins of the technology and/or the compositions comprising the same are administered according to a regime of treatment that is suitable for preventing and/or treating the disease and/or disorder to be prevented or treated. The clinician will generally be able to determine a suitable treatment regimen, depending on factors such as the stage of the disease and/or disorder to be treated, the severity of the disease and/or disorder to be treated and/or the severity of the symptoms thereof, the specific polypeptide, fusion protein or composition of the technology to be used, the specific route of administration and pharmaceutical formulation or composition to be used, the age, gender, weight, diet, general condition of the patient, and similar factors well known to the clinician.

Generally, the treatment regimen will comprise the administration of one or more polypeptides or fusion proteins of the technology, or of one or more compositions comprising the same, in one or more pharmaceutically effective amounts or doses. The specific amount(s) or doses to be administered can be determined by the clinician, again based on the factors cited above.

Usually, in the above method, a single polypeptide or fusion protein of the technology will be used. It is however within the scope of the technology to use two or more polypeptides, and/or fusion proteins of the technology in combination.

The polypeptides or fusion proteins of the technology may also be used in combination with one or more further pharmaceutically active compounds or principles, i.e., as a combined treatment regimen, which may or may not lead to a synergistic effect. Again, the clinician will be able to select such further compounds or principles, as well as a suitable combined treatment regimen, based on the factors cited above and his expert judgement.

In particular, the polypeptides or fusion proteins of the technology may be used in combination with other pharmaceutically active compounds or principles that are or can be used for the prevention and/or treatment of the disease and/or disorder cited herein, as a result of which a synergistic effect may or may not be obtained. Examples of such compounds and principles, as well as routes, methods and pharmaceutical formulations or compositions for administering them will be clear to the clinician.

When two or more substances or principles are to be used as part of a combined treatment regimen, they can be administered via the same route of administration or via different routes of administration, at essentially the same time or at different times (e.g., essentially simultaneously, consecutively, or according to an alternating regime). When the substances or principles are to be administered simultaneously via the same route of administration, they may be administered as different pharmaceutical formulations or compositions or part of a combined pharmaceutical formulation or composition, as will be clear to the skilled person.

In one aspect, the disclosure provides methods for the administration of the polypeptides, fusion proteins of the technology and/or compositions comprising the same. In some embodiments, polypeptides, fusion proteins of the technology and compositions comprising the same are administered as a pharmaceutical composition. The pharmaceutical composition, in addition to the polypeptides, fusion proteins of the technology and compositions comprising the same of include a pharmaceutically acceptable carrier.

Since the polypeptides, fusion proteins of the technology and compositions comprising the same have an increased half-life, they are preferably administered to the circulation. As such, they can be administered in any suitable manner that allows the compound or polypeptide of the technology to enter the circulation, such as intravenously, via injection or infusion, or in any other suitable manner (including oral administration, subcutaneous administration, intramuscular administration, administration through the skin, intranasal administration, administration via the lungs, etc.). Suitable methods and routes of administration will be clear to the skilled person, again for example also from the teaching of the published patent applications of Ablynx N.V., such as for example WO 04/041862, WO 2006/122786, WO 2008/020079, WO 2008/142164 or WO 2009/068627.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

Methods of preparing these formulations or compositions include the step of bringing into association an immunoglobulin single variable domain or polypeptide construct with the carrier and, optionally, one or more accessory ingredients. In general, the formulations are prepared by uniformly and intimately bringing into association an immunoglobulin single variable domain or polypeptide construct with liquid carriers, or finely divided solid carriers, or both, and then, if necessary, shaping the product.

The present technology further provides the polypeptides and/or fusion proteins and/or pharmaceutical composition of the technology as a vaccine, or as an immunogenic composition. Hence, the present technology further provides a vaccine comprising the polypeptide, fusion protein and/or pharmaceutical composition of the technology. The vaccine can be administered to the subject in need thereof as described above. For instance, the vaccine may be administered by mucosal administration, such as orally or intranasally.

The term “vaccine”, as used herein, refers to a substance or composition that establishes or improves immunity to a particular disease by inducing an adaptive immune response including an immunological memory, i.e., a substance or composition that, when administered to a subject in an effective amount, stimulates the production of protective antibody or protective T-cell response. Vaccines can be prophylactic or therapeutic. In one aspect, the vaccine of the present technology is a prophylactic vaccine.

The vaccine of the present technology may comprise an adjuvant. The term “adjuvant”, as used herein, refers to a substance which, when added to an immunogenic agent, non-specifically enhances or potentiates an immune response to the agent in a recipient host upon exposure to the mixture.

The Figures, Sequence Listing and the Experimental Part/Examples are only given to further illustrate the technology and should not be interpreted or construed as limiting the scope of the technology and/or of the appended claims in any way, unless explicitly indicated otherwise herein.

The embodiments illustrated and discussed in this specification are intended only to teach those skilled in the art the best way known to the inventors to make and use the technology. Modifications and variation of the above-described embodiments of the technology are possible without departing from the technology, as appreciated by those skilled in the art in light of the above teachings. It is therefore understood that, within the scope of the claims and their equivalents, the technology may be practiced otherwise than as specifically described.

The technology will now be further described by means of the following non-limiting preferred aspects, examples and figures.

The entire contents of all of the references (including literature references, issued patents, published patent applications, and co-pending patent applications) cited throughout this application are hereby expressly incorporated by reference, in particular for the teaching that is referenced hereinabove.

### Further Sequences

TABLE A
Sequences for CDRs according to AbM numbering
and frameworks (“ID” refers to the
given SEQ ID NO.)

| Name | ID | Amino acid sequence |
|---|---|---|
| ALB23002/Alb-1 CDR1 (AbM) | 1 | GFTFRSFGMS |
| ALB23002/Alb-1 CDR2(AbM) | 2 | SISGSGSDTL |
| ALB23002/Alb-1 | 3 | GGSLSR |
| CDR3(AbM/Kabat) | | |
| CDR1 Alb-binding ISVDs | 22 | GFTFSSFGMS |
| (AbM) | | |
| ALB23002 FR1 (AbM) | 23 | EVQLVESGGGVVQPGGSLRLSCAAS |
| ALB23002 FR2 (AbM) | 24 | WVRQAPGKGPEWVS |
| ALB23002 FR3 (AbM) | 25 | YADSVKGRFTISRDNSKNTLYLQMNSLRPEDTALYYCTI |
| ALB23002 FR4 (AbM/Kabat) | 26 | SSQGTLVTVSS |
| ALB23002/Alb1 CDR1 (Kabat) | 27 | SFGMS |
| ALB23002/Alb1 CDR2 (Kabat) | 28 | SISGSGSDTLYADSVKG |
| ALB23002 FR1 (Kabat) | 30 | EVQLVESGGGVVQPGGSLRLSCAASGFTFR |
| ALB23002 FR2 (Kabat) | 24 | WVRQAPGKGPEWVS |
| ALB23002 FR3 (Kabat) | 32 | RFTISRDNSKNTLYLQMNSLRPEDTALYYCTI |
| T023500029 CDR1 (Kabat) | 34 | TYVMG |
| T023500029 CDR2 (Kabat) | 35 | AISQNSIHTYYANSVKG |
| T023500029 CDR1 (Abm) | 36 | GGTFSTYVMG |
| T023500029 CDR2 (Abm) | 37 | AISQNSIHTY |
| T023500029 CDR3 | 38 | SRFTSWYTADYEYDY |
| (Kabat/Abm) | | |
| T023500029 FR1 (Abm) | 60 | EVQLVESGGGVVQPGDSLRLSCAAS |
| T023500029 FR2 (Abm) | 61 | WFRQAPGKEREFVS |
| T023500029 FR3 (Abm) | 62 | YANSVKGRFTISRDNSKNTVYLQLNSLRPEDTALYYCAA |
| T023500029 FR4 (Abm and | 63 | WGQGTLVTVSS |
| Kabat) | | |
| T023500029 FR1 (Kabat) | 64 | EVQLVESGGGVVQPGDSLRLSCAASGGTFS |
| T023500029 FR2 (Kabat) | 65 | WFRQAPGKEREFVS |
| T023500029 FR3 (Kabat) | 66 | RFTISRDNSKNTVYLQLNSLRPEDTALYYCAA |
| ALBX00002 CDR1 (AbM) | 55 | GLTFSSYAMG |
| ALBX00002 CDR2 (AbM) | 56 | SISRGGGYTY |
| ALBX00002 CDR3 (AbM and | 57 | ARYWATGSEYEFDY |
| Kabat) | | |
| ALBX00002 CDR1 (Kabat) | 58 | SYAMG |
| ALBX00002 CDR2 (Kabat) | 59 | SISRGGGYTYYADSVKG |
| ALBX00002 FR1 (AbM) | 23 | EVQLVESGGGVVQPGGSLRLSCAAS |
| ALBX00002 FR2 (AbM) | 68 | WFRQAPGKERERVV |
| ALBX00002 FR3 (AbM) | 69 | YADSVKGRFTISRDNSKNTVYLQMNSLRPEDTALYYCAA |
| ALBX00002 FR4 (AbM and | 70 | WGQGTLVTVSS |
| Kabat) | | |
| ALBX00002 FR1 (Kabat) | 71 | EVQLVESGGGVVQPGGSLRLSCAASGLTFS |
| ALBX00002 FR2 (Kabat) | 68 | WFRQAPGKERERVV |
| ALBX00002 FR3 (Kabat) | 73 | RFTISRDNSKNTVYLQMNSLRPEDTALYYCAA |

### EXAMPLES

### Example 1: Generation of Nanobody® ISVD Expression Constructs (Fusion Proteins), Protein Expression and Purification

Multivalent Nanobody® (ISVD) proteins (ISVD-comprising polypeptides, fusion proteins or constructs, as defined herein) were expressed in P. pastoris. The yeast expression vectors contain the AOX1 promoter and terminator, a resistance gene for Zeocin and the coding information for the Saccharomyces cerevisiae α-mating factor signal peptide. The ISVDs were combined with GS linkers (SEQ ID NO.: 45, 49 or 52) and cloned in the expression vector via Golden Gate cloning (Engler C, Marillonnet S. Golden Gate cloning. Methods Mol Biol. 2014; 1116:119-31). The expression vectors contain two Bpil restriction sites for the cloning the PCR-amplified monovalent ISVDs together with the GS linkers included in one or multiple vectors. All these elements are flanked by Bpil sites. The use of unique nucleotide overhangs for each position of the cloning cassette allows seamless ligation in a pre-defined order. After Sanger sequence confirmation, plasmid DNA derived from E. coli TOP10 was linearized and transformed by electroporation into in-house prepared hypercompetent P. pastoris, strain NRRL Y-11430 (ATCC 76273). The multivalent constructs containing at least one ALB23002 ISVD (SEQ ID NO.: 4) were purified on Amsphere A3 (JSR) or MabCaptureA (Poros) resins followed by a desalting step (PD columns with Sephadex G25 resin, GE Healthcare). Concentration was determined via OD280/OD340 measurement. Quality control was performed by SDS-PAGE and mass spectrometry.

### Example 2: Albumin Binding Studies of Nanobody ISVD Expression Constructs (Fusion Proteins)

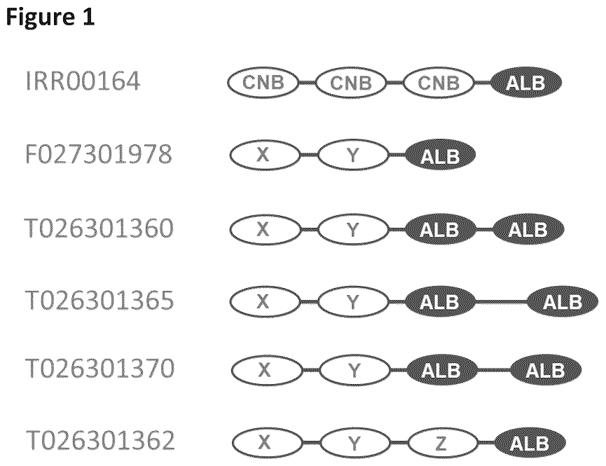

A set of ISVD expression constructs was generated that typically consisted of (i) one or two albumin binding ISVDs (ALB23002, SEQ ID NO.: 4) and (ii) two or three target ISVDs (X,Y,Z) to represent possible future therapeutic leads or three control ISVDs building blocks (“CNB”, also referred to as “IRR”) not binding to serum albumin or any other envisaged target but solely included in the polypeptide construct so as to create a similar size. See FIG. 1. The different ISVDs were fused with GS-linkers (as described in detail herein, see SEQ ID NO.: 45, 49 and 52) and a C-terminal Alanine was added.

We first assessed in SPR at pH 7,4 if simultaneous HSA binding was possible and if this was influenced by the linker length between the two albumin-binding building blocks. For this assessment, target X was immobilized on a CM5 (Cytiva, S series, BR100399) sensor via amine coupling using a Biacore 8k+ device. The formats were captured on this target via the first building block of the constructs, followed by an 120s injection of increasing concentrations of HSA, ranging from 10 nM to 30 μM in a MCK experiment. As control, a format with only 1 albumin-binding building block was included. The binding levels of HSA were significantly higher on the formats containing 2 ALB building blocks compared to the control. We estimated the amount of HSA molecules bound to the constructs comprising ISVD(s) (ISVD-comprising polypeptides or constructs), based on the ratio of the capture level to the binding level and the molecular weight of both the ISVD-comprising construct and HSA. When determining the theoretical number of HSA molecules bound, the number of HSA molecules bound increased with increasing HSA concentrations till saturation was reached. At saturation (reached at 30 μM HSA), we obtained a range of 1.45 to 1.69 theoretical HSA molecules if 2 albumin-binding building blocks are present, compared to a maximum of 0.93 for the ISVD-comprising construct with a single albumin-binding building block. This indicates that simultaneous HSA binding is possible if 2 albumin-binding building blocks are present, and this for all linker lengths tested (9GS, 20GS or 35GS, SEQ ID NO.: 45, 49 or 52, respectively) (see Table 7).

TABLE 7
Simultaneous HSA binding of ISVD-comprising constructs (fusion proteins) in SPR

| | | HSA concentration (μM) | | | | | | | | | |
|---|---|---|---|---|---|---|---|---|---|---|---|
| Construct ID | Format | 0.01 | 0.05 | 0.15 | 0.3 | 1 | 2.5 | 5 | 10 | 15 | 30 |
| T026301360 | X-9GS-Y-9GS- | 0.16 | 0.47 | 0.93 | 1.22 | 1.56 | 1.65 | 1.68 | 1.68 | 1,68 | 1.69 |
| | ALB23002- | | | | | | | | | | |
| | 9GS- Alb223 | | | | | | | | | | |
| T026301365 | X-9GS-Y-9GS- | 0.14 | 0.46 | 0.85 | 1.08 | 1.36 | 1.44 | 1.46 | 1.47 | 1.49 | 1.48 |
| | ALB23002- | | | | | | | | | | |
| | 35GS- Alb223 | | | | | | | | | | |
| T026301370 | X-9GS-Y-9GS- | 0.12 | 0.39 | 0.79 | 1.00 | 1.32 | 1.41 | 1.44 | 1.45 | 1.46 | 1.45 |
| | ALB23002- | | | | | | | | | | |
| | 2OGS- Alb223 | | | | | | | | | | |
| T026301362 | X-9GS-Y-9GS- | 0.09 | 0.25 | 0.49 | 0.65 | 0.83 | 0.89 | 0.91 | 0.92 | 0.93 | 0.93 |
| | Z-9GS-Alb223 | | | | | | | | | | |

The constructs containing a 9GS and 35GS linker between the albumin-binding building blocks were selected for further characterization. The affinities of the purified ISVD expression constructs for human and mouse serum albumin (HSA and MSA, respectively) at pH 6.0 and pH 7.4 were determined on a Biacore 8K+ instrument. HSA or MSA (HSA: Sigma-Aldrich-Sigma, Cat No. A8763; MSA: Albumin Bioscience, Cat No. 2601) was immobilized on a Series S Sensor Chip C1. The fusion proteins as described in Table 7 above were injected at 9 different concentrations (between 0.6 and 2000 nM) and allowed to associate for 120 s at 30 μL/min and dissociate for 600 s. Evaluation of the sensorgrams was based on the bivalent analyte fit. The affinities for HSA and MSA are shown in Table 8.

TABLE 8
HSA and MSA binding of Nanobody ® ISVD constructs (fusion proteins) at pH 6.0 and pH 7.4

| | | | Affinity | | | | | |
|---|---|---|---|---|---|---|---|---|
| | | | ka1 | kd1 | KD1 | ka2 | kd2 | KD2 |
| NB ID | Target | PH | 1/Ms | 1/s | M | 1/Ms | 1/s | M |
| T026301365 | HSA | pH 7.4 | 1.6E+05 | 1.6E−03 | 9.9E−09 | 8.4E−04 | 4.5E−03 | 5.3E+00 |
| T026301360 | HSA | pH 7.4 | 1.2E+05 | 1.5E−03 | 1.2E−08 | 5.3E−04 | 2.6E−03 | 4.8E+00 |
| T026301365 | HSA | pH 6.0 | 1.8E+05 | 1.6E−03 | 8.8E−09 | 2.8E−04 | 2.1E−03 | 7.4E+00 |
| T026301360 | HSA | pH 6.0 | 1.7E+05 | 1.5E−03 | 9.0E−09 | 4.5E−04 | 2.8E−03 | 6.3E+00 |
| T026301365 | MSA | pH 7.4 | 3.2E+04 | 4.3E−03 | 1.4E−07 | 7.3E+00 | 7.1E+00 | 9.8E−01 |
| T026301360 | MSA | pH 7.4 | 1.7E+05 | 3.4E−02 | 2.1E−07 | 1.1E−04 | 2.2E−04 | 2.0E+00 |
| T026301365 | MSA | pH 6.0 | 5.8E+04 | 5.9E−03 | 1.0E−07 | 7.8E+00 | 1.9E+01 | 2.5E+00 |
| T026301360 | MSA | pH 6.0 | 1.9E+05 | 4.0E−02 | 2.1E−07 | 9.9E−05 | 3.9E−04 | 3.9E+00 |

Italics and underlined: Indicative values

### Example 3: Development and Optimization of Serum PK Assays

A specific and sensitive ligand binding assay was developed to measure concentrations of I1RR0064, F027301978, T026301360 and T026301365 in mouse serum. A streptavidin-coated MSD GOLD 96-well SMALLSPOT® plate (Meso Scale Discovery) was blocked with Superblock T20™ (Thermo Scientific) for 30 minutes at RT. The plate was then washed and incubated for 1 hour at RT and at 600 rpm with 1.0 μg/mL biotinylated generic mAb directed against the frameworks of the different ISVD building blocks. Calibrators and QCs were prepared in pooled mouse serum. After washing the plate, calibrators, QCs and samples were applied to the plate at an MRD of 20 in PBS 0.1% casein and incubated for 1 hour at RT and at 600 rpm. After washing, the plate was incubated for 1 hour at RT and at 600 rpm with 2.0 μg/mL sulfo-labelled mAb directed against a specific ISVD building block, depending on the construct under evaluation. After the plate was washed, 2×MSD Read buffer (Meso Scale Discovery) was added and the plate was read on a Sector Imager Quickplex SQ 120 (Meso scale Discovery).

The same setup was used to measure the concentration of F027301978 and T026301360 in cynomolgus monkeys, with following changes: the calibrators and QCs were prepared in pooled cynomolgus monkey serum and the applied MRD was 100.

### Example 4: Pharmacokinetics in Mice of Fusion Proteins Comprising One or Two Serum Albumin Binding ISVD (ALB23002), and Additional Target Binding ISVD Building Blocks (X,Y)

Pharmacokinetic experiments were performed in TG32 (B6.Cg-Fcgrttm1Dcr Tg(FCGRT) 32Dcr/Dcr) mice to evaluate half-life of fusion proteins (multivalent ISVD-comprising constructs) consisting of two target ISVD building blocks (X,Y) fused to either a single albumin-binding ISVD Alb23002, SEQ ID NO.: 4 (trivalent construct) or to 2 Alb23002 ISVD, SEQ ID NO.: 4 (tetravalent constructs). A construct containing three non-targeting (control) (CNB) ISVD fused to ALB23002, SEQ ID NO.: 4 was evaluated as a tetravalent control construct (IRR00164). All constructs comprise a C-terminal A, see Table 9.

To mimic relevant competition with hIgG, Tg32 mice were preloaded with a mixture of purified hIgG (hIVIG; Privigen®). Privigen® was administered intravenously once weekly, with the first administration 2 days prior to initiation of the PK study. In total, 3 Privigen® injections of 250 mg/kg were administered, yielding physiologically relevant hIgG serum concentrations for the duration of the study (data not shown). Two days after the first Privigen® administration, 4 to 6 Tg32 mice per group were injected intravenously in the tail with equimolar amounts (3.5 mg/kg for tetravalent constructs or 2.7 mg/kg for trivalent construct (constructs and building blocks used in these constructs were described in Example 1 above, see also Table 9 below).

Blood was retrieved at different time points (2 mice per time point) and serum was prepared. Serum samples were analyzed by ELISA for the presence of multivalent ISVD constructs as described in Example 3. All in vivo studies were conducted in compliance with the Sanofi institutional animal care policy.

Results are shown in FIG. 2. Half-lives values were obtained by estimating the in vivo endosomal FcRn affinities in a mechanistic PBPK model and reported in Table 9.

In multivalent ISVD-comprising constructs comprising ISVD domains against therapeutic targets (X, Y), the combination of two albumin binding ISVD significantly improved half-life compared to the control ISVD-comprising constructs containing only a single albumin binding ISVD. Linker length between both albumin-binding ISVDs did not significantly impact the pharmacokinetic properties (half-life) of the constructs. In addition, half-life was not impacted by the presence of relevant levels of hIgG. For the constructs with 2 albumin-binding ISVDs, half-life was improved up to 1.8- to 2.8-fold compared to the trivalent and tetravalent control ISVD-comprising constructs containing a single albumin-binding ISVD, respectively.

TABLE 9
Formats and calculated half-lives of fusion proteins
(multivalent ISVD constructs) evaluated in Tg32 mice.

| | IRR00164 | F027301978 | T026301360 | T026301365 |
|---|---|---|---|---|
| Format | CNB-CNB- | X-Y- | X-Y- | X-Y- |
| | CNB- | Alb223 | Alb23002- | Alb23002- |
| | Alb223 | | Alb223 | Alb223 |
| Linker length | NA | NA | 9 GS | 35 GS |
| between | | | | |
| Alb-Alb | | | | |
| T_{1/2} (days) | 1.5 | 2.4 | 3.8 | 4.3 |

### Example 5: Pharmacokinetics in Cynomolgus Monkeys of Fusion Proteins Comprising One or Two Serum Albumin Binding ISVD (ALB23002) and Additional Target Binding ISVD Building Blocks (X,Y)

A Pharmacokinetic study in cynomolgus monkeys was conducted with two of the multivalent ISVD-comprising constructs described above, F027301987 (trivalent construct with single albumin binding ISVD, Alb223, SEQ ID NO.: 18, X-Y-Alb223) and T026301360 (tetravalent construct comprising two albumin-binding ISVDs, Alb23002, SEQ ID NO.: 4 and Alb223, SEQ ID NO.: 18, X-Y-Alb23002-Alb223). Four healthy male cynomolgus monkeys (2 animals per compound) were dosed with a single-dose of one of the test constructs at 10 mg/kg via IV administration, followed by a 2-month observation period. Blood sampling for PK analysis was performed at multiple time points (2 animals per time point) and serum was prepared. Serum samples were analyzed by ELISA for the presence of multivalent constructs as described in Example 3.

PK parameters were obtained from non-compartmental analysis in Phoenix WinNonlin® (version 8.2.2.227, Certara) using the Plasma Data Module. PK profiles from animal 8 (F027301987 group) showed a sharp drop in concentration, typical of anti-drug antibody (ADA) interference. This was confirmed by ADA analysis and the PK profile was excluded from PK calculations. Comparison in PK parameters (relative difference compared to F027301978) is reported in Table 10.

In cynomolgus monkeys, the construct with double albumin binding ISVD (T026301360) displayed reduced clearance and improved serum half-life (T_{1/2}) compared to the construct with single albumin binding ISVD (F027301978). For T026301360, the mean of 2 animals was used to calculate the relative difference to F027301978 (n=1).

TABLE 10
Comparison in PK parameters of multivalent constructs
(relative difference compared to F027301978)

| | | Clearance | T_{1/2} |
|---|---|---|---|
| TEST | | (% vs | (% vs |
| CONSTRUCT | Animals | F027301978) | F027301978) |
| F027301978 | N = 1 | 100% | 100% |
| T026301360 | N = 2 | 39% | 138% |

### Example 6: Generation of Multivalent Albumin Binding Expression Constructs, Protein Expression and Purification

A set of ISVD-comprising constructs was generated that typically consisted of (i) one to four albumin binding ISVD(s) (ALB23002, SEQ ID NO.: 4; HSA006A06, SEQ ID NO.: 91; ALBX00002, SEQ ID NO.: 54 or T023500029, SEQ ID NO.: 20) and (ii) no or one other albumin binding building block (DARPIN, SEQ ID NO.: 88 or ABD, SEQ ID NO.: 90). The different albumin binding building blocks were fused with 35 GS-linkers (as described in detail herein, see SEQ ID NO.: 52) and a C-terminal Alanine was added if the C-terminal building block was an ISVD. Multivalent albumin binding constructs (fusion proteins or constructs) were expressed in CHO and purified from the cell supernatants using a protein A capture step followed by a size exclusion chromatography purification step.

### Example 7: Albumin Binding Studies of Multivalent Albumin Binding Expression Constructs

In a first experiment, the affinities of monovalent (ALB00223) and a bivalent ALB23002 construct (T032200003) for human and mouse serum albumin (HSA and MSA, respectively) at pH 6.0 and pH 7.4 were determined on a ProteOn XPR36 instrument (Bio-rad Laboratories, Inc.). HSA or MSA (HSA: Sigma-Aldrich—Sigma, Cat No. A8763; MSA: Albumin Bioscience, Cat No. 2601) was immobilized via amine coupling on a GLC sensor (short matrix, normal capacity). The constructs were injected at 6 different concentrations (between 0.95 and 500 nM) and allowed to associate for 120 s at 45 μL/min and dissociate for 600 s. Evaluation of the sensorgrams was based on the Kinetic Langmuir 1:1 (simultaneous ka/kd) fit. The affinities for HSA and MSA are shown in Table 11.

For the different multivalent albumin-binding constructs shown in FIG. 3, simultaneous HSA binding at pH 7,4 was assessed by SPR.

The constructs with a C-terminal Alanine were captured on the chip via an anti-ISVD antibody that was captured on the chip (CM5 (Cytiva, S series, BR100399) by its Fc chain. Next, increasing concentrations of HSA from 10 nM to 100 μM were injected during 120s in a MCK experiment. As a control, a monovalent ALB23002-derived construct (Alb223, SEQ ID NO.: 18) was used. The binding levels of HSA were significantly higher on the constructs containing multiple albumin binding building blocks compared to the control. We estimated the amount of HSA molecules bound to the ISVD-comprising constructs, based on the ratio of the capture level to the binding level and the molecular weight of both the construct and HSA. When determining the theoretical number of HSA molecules bound, the number increased with increasing HSA concentrations until saturation, and all constructs with more than one albumin binding building block could bind also more than one HSA molecule. For the constructs containing ALB23002 (or Alb223, which corresponds to ALB23002 with an additional C-terminal A) or HSA006A06 (which are variants of the same parental ISVD) or DARPIN, we obtained a range of 1.6 to 1.7 HSA molecules for the bivalent constructs, 2.4 to 2.5 for the trivalent constructs and 3.2 for the tetravalent construct at the highest HSA concentration (100 μM). These results indicate that simultaneous HSA binding is possible on all albumin-binding building blocks that are comprised in the construct (see Table 12). For the bivalent constructs containing ALBX00002, T023500029 or ABD we calculated a binding of 1.1 HSA molecules per construct, at the highest HSA concentration (100 μM), which is higher than the 0.8 HSA molecules obtained for ALB23002-A.

TABLE 11
Affinities, on and off-rates of constructs comprising a single albumin-binding ISVD or a
bivalent albumin-binding ISVD, for human and mouse serum albumin at pH 6.0 and pH 7.4

| | | Affinity | | | | | |
|---|---|---|---|---|---|---|---|
| | | HSA | | | MSA | | |
| CONSTRUCT | | ka | kd | KD | ka | Kd | KD |
| ID | pH | 1/Ms | 1/5 | M | 1/Ms | 1/s | M |
| ALB00223 | pH 7.4 | 5.92E+05 | 8.09E−04 | 1.37E−09 | 7.67E+05 | 6.97E−03 | 9.09E−09 |
| ALB00223 | pH 6.0 | 8.53E+05 | 5.93E−04 | 6.9E−10 | 1.03E+06 | 6.18E−03 | 6.02E−09 |
| T032200003 | pH 7.4 | 5.33E+05 | 4.61E−04 | 8.65E−10 | 8.54E+05 | 1.67E−03 | 1.96E−09 |
| T032200003 | pH 6.0 | 7.59E+05 | 3.88E−04 | 5.12E−10 | 1.02E+06 | 1.64E−03 | 1.61E−09 |

Italics and underlined: indicative values

TABLE 12
Simultaneous HSA binding of multivalent albumin binding expression constructs in SPR
(number of HSA molecules bound per construct)

| | | HSA concentration | | | | | | | | | | | |
|---|---|---|---|---|---|---|---|---|---|---|---|---|---|
| | | 10 | 50 | 150 | 300 | 1 | 2.5 | 5 | 10 | 15 | 30 | 70 | 100 |
| Construct ID | Description | nM | nM | nM | nM | μM | μM | μM | μM | μM | μM | μM | μM |
| ALB00223-A | ALB23002-A (SEQ ID NO.: 18) | 0.2 | 0.4 | 0.5 | 0.6 | 0.7 | 0.8 | 0.8 | 0.8 | 0.8 | 0.8 | 0.8 | 0.8 |
| or Alb223 | | | | | | | | | | | | | |
| TPP-66090 | HSA006A06-35GS-HSA006A06-A (SEQ ID NO.: 74) | 0.4 | 0.8 | 1.2 | 1.4 | 1.6 | 1.7 | 1.8 | 1.8 | 1.8 | 1.8 | 1.7 | 1.7 |
| TPP-66092 | HSA006A06-35GS-ALB23002-A (SEQ ID NO.: 76) | 0.3 | 0.7 | 1.1 | 1.3 | 1.5 | 1.6 | 1.7 | 1.7 | 1.7 | 1.7 | 1.7 | 1.7 |
| TPP-66093 | ALBX00002-35GS-ALB23002-A (SEQ ID NO.: 77) | 0.2 | 0.4 | 0.6 | 0.7 | 0.8 | 0.9 | 0.9 | 0.9 | 1.0 | 1.0 | 1.1 | 1.1 |
| TPP-66094 | T023500029-35GS-ALB23002-A (SEQ ID NO.: 78) | 0.2 | 0.5 | 0.7 | 0.8 | 0.9 | 0.9 | 0.9 | 1.0 | 1.0 | 1.0 | 1.1 | 1.1 |
| TPP-66095 | DARPIN-35GS-ALB23002-A (SEQ ID NO.: 79) | 0.2 | 0.6 | 0.9 | 1.1 | 1.4 | 1.5 | 1.6 | 1.7 | 1.7 | 1.7 | 1.6 | 1.6 |
| TPP-66097 | ABD-35GS-ALB23002-A (SEQ ID NO.: 81) | 0.2 | 0.5 | 0.7 | 0.8 | 0.9 | 0.9 | 0.9 | 1.0 | 1.0 | 1.0 | 1.1 | 1.1 |
| TPP-66099 | ALB23002-35GS-ALB23002-35GS-ALB23002-A | 0.4 | 1.1 | 1.6 | 1.9 | 2.3 | 2.4 | 2.5 | 2.5 | 2.5 | 2.5 | 2.5 | 2.4 |
| | (SEQ ID NO.: 82) | | | | | | | | | | | | |
| TPP-66100 | ALBX00002-35GS-T023500029-35GS-ALB23002-A | 0.3 | 0.8 | 1.1 | 1.3 | 1.6 | 1.7 | 1.7 | 1.7 | 1.7 | 1.7 | 1.7 | 1.7 |
| | (SEQ ID NO.: 83) | | | | | | | | | | | | |
| TPP-66101 | DARPIN-35GS-ALB23002-35GS-ALB23002-A | 0.4 | 0.9 | 1.5 | 1.8 | 2.2 | 2.4 | 2.5 | 2.5 | 2.5 | 2.5 | 2.5 | 2.5 |
| | (SEQ ID NO : 84) | | | | | | | | | | | | |
| TPP-66102 | ALB23002-35GS-ALB23002-35GS-ALB23002-35GS- | 0.5 | 1.3 | 2.1 | 2.6 | 3.0 | 3.2 | 3.3 | 3.3 | 3.3 | 3.3 | 3.2 | 3.2 |
| | ALB23002-A (SEQ ID NO.: 85) | | | | | | | | | | | | |

| | | |
|---|---|---|
| TPP-128760 | ALB23002-35GS-DARPIN (SEQ ID NO.: 150) | ND |
| TPP-128762 | ALB23002-35GS-ABD (SEQ ID NO.: 151) | ND |

ND: not determined

The affinities of the purified multivalent albumin binding constructs for HSA at pH 7.4 was determined on a Biacore 8K+ instrument. HSA (Sigma-Aldrich—Sigma, Cat No. A8763) was immobilized on a Series S Sensor Chip C1. The multivalent albumin binding proteins as described in Table 12 above were injected at 9 different concentrations (between 1.6 and 2500 nM) and allowed to associate for 120 s at 30 μL/min and dissociate for 600 s. Evaluation of the sensorgrams was based on the bivalent analyte fit and the affinities for HSA are shown in Table 13.

TABLE 13
HSA affinities of multivalent albumin binding constructs at pH 7.4

| | Affinity for HSA | | | | | |
|---|---|---|---|---|---|---|
| | ka1 | kd1 | KD1 | ka2 | kd2 | KD2 |
| Construct ID | 1/Ms | 1/s | M | 1/Ms | 1/s | M |
| TPP-66090 | 5.2E+07 | 3.1E−03 | 6.0E−11 | 2.8E−02 | 6.8E−04 | 2.4E−02 |
| TPP-66092 | 2.9E+05 | 4.0E−04 | 1.4E−09 | 6.6E−04 | 1.6E−03 | 2.4E+00 |
| TPP-66093 | 1.8E+05 | 1.2E−04 | 6.6E−10 | 1.2E−04 | 8.4E−04 | 7.0E+00 |
| TPP-66094 | 2.7E+05 | 1.3E−04 | 4.9E−10 | 2.7E−04 | 2.2E−03 | 8.1E+00 |
| TPP-66095 | No good fit possible | | | | | |
| TPP-66097 | 1.8E+05 | 1.5E−05 | 8.2E−11 | 2.3E−03 | 8.8E−03 | 3.9E+00 |
| TPP-66099 | 3.7E+05 | 5.0E−04 | 1.4E−09 | 2.2E−03 | 3.8E−03 | 1.7E+00 |
| TPP-66100 | 2.0E+05 | 3.4E−04 | 1.7E−09 | 7.4E−03 | 2.6E−02 | 3.5E+00 |
| TPP-66101 | 3.0E+05 | 1.5E−03 | 5.0E−09 | 9.5E+01 | 6.5E+01 | 6.9E−01 |
| TPP-66102 | 2.8E+05 | 5.5E−04 | 2.0E−09 | 5.0E−03 | 6.6E−03 | 1.3E+00 |
| TPP-128760 | 1.8E+05 | 2.9E−03 | 1.6E−08 | 1.4E−03 | 6.1E−04 | 4.3E−01 |
| TPP-128762 | 2.6E+06 | <3.70E−04 | <1.44E−10 | 3.9E−03 | 4.0E−03 | 1.0E+00 |

Italics and underlined: indicative values

The purified multivalent albumin binding constructs were also tested in SPR on different densities of HSA (100 RU until 6700 RU) to detect avidity. Off-rates were determined at pH 7.4 and are shown in Table 14. These results show that the off-rates get slower if the HSA density is increased, for constructs containing 2 or more albumin binding domains, except for TPP-66097 and TPP-66100. The simultaneous HSA binding experiment in Table 13 suggests that these constructs can bind more than one HSA molecule at 100 μM HSA.

TABLE 14
Off-rates of constructs determined at increasing density of HSA

| | | HSA density (RU) | | | |
|---|---|---|---|---|---|
| | | ~100 | ~500 | ~3000 | ~6700 |
| Construct ID | Description | kd (1/s) | kd (1/s) | kd (1/s) | kd (1/s) |
| IRR00164 | CNB-9GS-CNB-9GS- | ND | 2.0E−03 | 1.6E−03 | 1.2E−03 |
| | CNB-9GS-ALB23002-A | | | | |
| TPP-66090 | HSA006A06-35GS- | 3.8E−04 | 1.9E−04 | 8.7E−05 | 4.5E−05 |
| | HHSA006A06-A | | | | |
| TPP-66092 | HSA006A06-35GS- | 3.8E−04 | 2.0E−04 | 9.0E−05 | 4.8E−05 |
| | ALB23002-A | | | | |
| TPP-66093 | ALBX00002-335GS- | 1.1E−04 | 8.6E−05 | 6.6E−05 | 5.2E−05 |
| | ALB23002-A | | | | |
| TPP-66094 | T023500029-35GS- | 1.2E−04 | 9.3E−05 | 7.6E−05 | 5.6E−05 |
| | ALB23002-A | | | | |
| TPP-66095 | DARPIN-35GS- | 2.1E−04 | 3.8E−04 | 1.6E−04 | 8.3E−05 |
| | ALB23002-A | | | | |
| TPP-66097 | ABD-35GS- | 7.4E−05 | 1.2E−04 | 8.4E−05 | 6.0E−05 |
| | ALB23002-A | | | | |
| TPP-66099 | ALB23002-35GS- | 4.1E−04 | 1.8E−04 | 7.2E−05 | 3.6E−05 |
| | ALB23002-35GS- | | | | |
| | ALB23002-A | | | | |
| TPP-66100 | ALBX00002-35GS- | 2.7E−04 | 3.2E−04 | 3.6E−04 | 3.2E−04 |
| | T023500029-35GS- | | | | |
| | ALB23002-A | | | | |
| TPP-66101 | DARPIN-35GS- | 7.6E−04 | 2.3E−04 | 1.0E−04 | 4.0E−05 |
| | ALB23002-35GS- | | | | |
| | ALB23002-A | | | | |
| TPP-66102 | ALB23002-35GS- | 4.3E−04 | 1.2E−04 | 3.6E−05 | 1.4E−05 |
| | ALB23002-35GS- | | | | |
| | ALB23002-35GS- | | | | |
| | ALB23002-A | | | | |
| TPP- | ALB23002-35GS- | ND | ND | ND | ND |
| 128760 | DARPIN | | | | |
| TPP- | ALB23002-35GS-ABD | ND | ND | ND | ND |
| 128762 | | | | | |

ND: not determined

### Example 8: Development and Optimization of Mouse PK Assays

A bottom-up LC-MS2 assay was developed to measure concentration of constructs comprising multiple serum albumin binding domains-comprising constructs in mouse plasma. As internal standard, an in-house produced fusion Nanobody construct was used.

Briefly, samples were diluted, reduced, carbamidomethylated and proteins were precipitated. The obtained pellets were digested with trypsin and the resulting surrogate peptides were analysed performed in an Agilent 1290 Infinity II UHPLC hyphenated to a Sciex 6500+ mass spectrometer. For separation, a column Aquity UPLC Peptide CSH C18 130 Å 1.7 μm 50×2.1 mm (Waters) was flushed at 50° C. with a stepwise gradient of water/DMSO/formic acid (100/1/0.5; v/v/v) and acetonitrile/DMSO/formic acid (100/1/0.5; v/v/v) with a 0.50 mL min-1 flow. The mass spectrometer was operated in positive mode according manufacturer's instructions. One multiple reaction was monitored for each construct. Chromatographic peak areas were determined with the algorithm AutoPeak (Sciex OS). Concentrations were determined by using the ratio area of the analyte to the area of the internal standard in the same sample and comparing the results to the calibration curve obtained with the calibration standards.

For one construct (T032200003), consisting of 2 ALB23002 building blocks separated by a 35 GS linker, (ALB23002-35GS-ALB23002, SEQ ID NO.: 152) a specific and sensitive ligand-binding assay was developed to measure concentrations in mouse serum. Briefly, a streptavidin-coated MSD GOLD 96-well SMALLSPOT® plate (Meso Scale Discovery) was blocked with Superblock T20TM (Thermo Scientific) for 30 minutes at RT. The plate was then washed and incubated for 1 hour at RT and at 600 rpm with 2.0 g/mL biotinylated generic mAb directed against the frameworks of the ISVD building blocks. Calibrators and QCs were prepared in pooled mouse serum. After washing the plate, calibrators, QCs and samples were applied to the plate at an MRD of 40 in PBS 0.1% casein and incubated for 1 hour at RT and at 600 rpm. After washing, the plate was incubated for 1 hour at RT and at 600 rpm with 4.0 g/mL sulfo-labelled mAb directed against another epitope of the ISVD building blocks. After the plate was washed, 2×MSD Read buffer (Meso Scale Discovery) was added and the plate was read on a Sector Imager Quickplex SQ 120 (Meso scale Discovery).

### Example 9: Pharmacokinetic Evaluation in BALB/c Mice of Constructs Comprising Multiple Serum Albumin Binding Domains

In a first PK study, T032200003, consisting of 2 ALB23002 building blocks separated by a 35GS linker was evaluated. BALB/c mice (n=3) were injected with 5 mg/kg of T032200003 (189 nmol/kg) and serum samples were obtained at 0.083, 0.5, 1, 2, 6, 24, 48, 96 and 168 h. Serum samples were analyzed by ligand-binding assay.

A second set of pharmacokinetic experiments was performed in BALB/c mice to evaluate half-life of a broad set of multivalent serum albumin binding-comprising constructs. Mice (n=3/group) were injected intravenously in the tail vein with an equimolar dose (approximately 189 nmol/kg) of the constructs as listed in Table 15. Plasma samples were obtained via serial (^{˜}10 uL) at 0.25, 1, 4, 8, 24, 48, 72, 168, 336 and 504 h.

Plasma samples were analyzed by LC-MS2 for the presence of multivalent ISVD-comprising constructs as described in Example 3. All in vivo studies were conducted in compliance with the Sanofi institutional animal care policy.

PK parameters for all constructs were obtained from non-compartmental analysis (NCA) in Phoenix WinNonlin® (version 8.2.2.227. Certara) using the Plasma Data Module. Where applicable, sampling times with steep concentration decline of compound due to suspected ADA impact were excluded from analysis. Results are reported in Table 15.

In multivalent constructs comprising of 2 or more albumin-binding domains, PK parameters are significantly improved compared to reported values of constructs containing only a single albumin binding domain (t_{1/2} range of 0.5 to 1.4 days), which corresponds to the half-life of albumin in wild type mice of approximately 1.5 days (35 h; Chaudhury et ol. 2003, J Exp Med. 2003 Feb. 3; 197(3):315-22). On average, for the multivalent albumin-binding constructs, half-life was improved to 3 days (range 2.4-3.6), which is approximately a 2-fold improvement compared to what is reported for molecules containing a single albumin-binding domain and to our internal experience with half-life extended (via albumin-binding) ISVDs.

TABLE 15
Dose and PK parameters of multivalent albumin-binding constructs evaluated in
BALB/c mice

| | | Dose | Dose | Cl | t½ |
|---|---|---|---|---|---|
| Construct ID | Format | (mg/kg) | (nmol/kg) | (mL/hr/kg) | (hr) |
| T032200003* | ALB23002-35GS-ALB23002 | 5 | 189 | 0.666 | 74.8 |
| | (SEQ ID NO.: 152) | | | | |
| TPP-66090 | HSA006A06-35GS-HSA006A06-A | 5 | 192 | 0.565 | 75.9 |
| TPP-66092 | HSA006A06-35GS-ALB23002-A | 5 | 188 | 0.559 | 82.8 |
| TPP-66093 | ALBX00002-35GS-ALB23002-A | 5.3 | 190 | 0.505 | 74.3 |
| TPP-66094 | T023500029-35GS-ALB23002-A | 5.3 | 188 | 0.481 | 60.5 |
| TPP-66099 | ALB23002-35GS-ALB23002- | 7.7 | 188 | 0.534 | 77.4 |
| | 35GS-ALB23002-A | | | | |
| TPP-66100 | ALBX00002-35GS-T023500029- | 8.3 | 189 | 0.598 | 58.3 |
| | 35GS-ALB23002-A | | | | |
| TPP-66101 | DARPIN-35GS-ALB23002-35GS- | 7.9 | 189 | 0.428 | 75.2 |
| | ALB23002-A | | | | |
| TPP-66102 | ALB23002-35GS-ALB23002- | 10.4 | 188 | 0.443 | 86.2 |
| | 35GS-ALB23002-35GS- | | | | |
| | ALB23002-A | | | | |
| TPP-128760 | ALB23002-35GS-DARPIN | 5.2 | 190 | 0.582 | 59.9 |
| TPP-128762 | ALB23002-35GS-ABD | 3.7 | 191 | 0.510 | 80.4 |

*T032200003 was evaluated in a separate study.

### ITEMS OF THE PRESENT TECHNOLOGY

1. A polypeptide comprising:
(i) at least one immunoglobulin single variable domain (ISVD) which specifically binds to a serum albumin protein; and
(ii) at least one further moiety specifically binding to a serum albumin protein or at least one human serum albumin protein,
wherein said (i) at least one ISVD essentially consists of 4 framework regions (FR1 to FR4, respectively) and three complementarity determining regions (CDR1 to CDR3, respectively), wherein:
a) CDR1 comprises the amino acid sequence of SEQ ID NO: 1 or has 3, 2 or 1 amino acid difference(s) with SEQ ID NO: 1 or CDR1 comprises the amino acid sequence of SEQ ID NO: 22 or has 3, 2 or 1 amino acid difference(s) with SEQ ID NO: 22
b) CDR2 comprises the amino acid sequence of SEQ ID NO: 2 or has 3, 2 or 1 amino acid difference(s) with SEQ ID NO: 2; and
c) CDR3 comprises the amino acid sequence of SEQ ID NO: 3 or has 3, 2 or 1 amino acid difference(s) with SEQ ID NO: 3,
and/or
a) CDR1 comprises the amino acid sequence of SEQ ID NO: 36 or has 3, 2 or 1 amino acid difference(s) with SEQ ID NO: 36;
b) CDR2 comprises the amino acid sequence of SEQ ID NO: 37 or has 3, 2 or 1 amino acid difference(s) with SEQ ID NO: 37; and
c) CDR3 comprises the amino acid sequence of SEQ ID NO: 38 or has 3, 2 or 1 amino acid difference(s) with SEQ ID NO: 38,
and/or
a) CDR1 comprises the amino acid sequence of SEQ ID NO: 55 or has 3, 2 or 1 amino acid difference(s) with SEQ ID NO: 55;
b) CDR2 comprises the amino acid sequence of SEQ ID NO: 56 or has 3, 2 or 1 amino acid difference(s) with SEQ ID NO: 56; and
c) CDR3 comprises the amino acid sequence of SEQ ID NO: 57 or has 3, 2 or 1 amino acid difference(s) with SEQ ID NO: 57
wherein the CDR sequences are determined according to AbM numbering.

2. The polypeptide according to item 1, wherein the (i) at least one ISVD specifically binding to a serum albumin protein comprises a CDR1 that comprises the amino acid sequence of SEQ ID NO: 1 or 22, a CDR2 that comprises the amino acid sequence of SEQ ID NO: 2; and a CDR3 that comprises the amino acid sequence of SEQ ID NO: 3, wherein the CDR sequences are determined according to AbM numbering.

3. The polypeptide according to any one of items 1 or 2, wherein the (i) at least one ISVD specifically binding to a serum albumin protein comprises a CDR1 that comprises the amino acid sequence of SEQ ID NO: 36, a CDR2 that comprises the amino acid sequence of SEQ ID NO: 37; and a CDR3 that comprises the amino acid sequence of SEQ ID NO: 38, wherein the CDR sequences are determined according to AbM numbering, or wherein the (i) at least one ISVD specifically binding to a serum albumin protein comprises a CDR1 that comprises the amino acid sequence of SEQ ID NO: 55, a CDR2 that comprises the amino acid sequence of SEQ ID NO: 56; and a CDR3 that comprises the amino acid sequence of SEQ ID NO: 57, wherein the CDR sequences are determined according to AbM numbering.

4. The polypeptide according to any one of items 1 to 3, wherein the (i) at least one ISVD specifically binding to a serum albumin protein has:
a) a degree of sequence identity with any one of the sequences as defined in SEQ ID NO's: 4 to 21, 54, or 91 to 93 of at least 85%, preferably at least 90%, more preferably at least 95%, in which any C-terminal extension that may be present as well as the CDRs are not taken into account for determining the degree of sequence identity; and/or
b) no more than 7, preferably no more than 5, such as only 3, 2 or 1 amino acid differences with any one of the sequences as defined in SEQ ID NO's: 4 to 21, 54, or 91 to 93.

5. The polypeptide according to according to any one of items 1 to 4, wherein the (i) at least one ISVD specifically binding to a serum albumin protein has a sequence that is chosen from SEQ ID NO's: 4 to 21, 54, or 91 to 93.

6. The polypeptide according to any one of items 1 to 5, wherein the amino acid sequence of the (i) at least one ISVD specifically binding to a serum albumin protein has a sequence identity of more than 90% with SEQ ID NO: 4 or SEQ ID NO.: 18.

7. The polypeptide according to any one of items 1 to 6, wherein the (i) at least one ISVDs specifically binding to a serum albumin protein comprises or consists of the amino acid sequence of Alb-23002 (SEQ ID NO: 4).

8. The polypeptide according to any one of items 1 to 7, wherein the (i) at least one ISVD specifically binding to a serum albumin protein specifically binds to serum albumin with a dissociation constant (K_{D}) of between 10⁻⁶ M and 10⁻¹¹ M or less, preferably of between 10⁻⁷ M and 10⁻¹² M or less, as determined using Proteon, Kinexa, BLI or SPR.

9. The polypeptide according to any one of items 1 to 8, wherein the (i) at least one ISVDs comprised therein is derived from a V_{H}, a humanized V_{H}, a human V_{H}, a V_{HH}, a humanized V_{HH} or a camelized V_{H} (derived from a heavy-chain ISVD), preferably derived from an ISVD belonging to the “V_{H}3 class”.

10. The polypeptide according to any one of items 1 to 9, wherein the (i) at least one ISVD specifically binding to a serum albumin protein contains, compared to any of the sequences of SEQ ID NO's: 4 to 21, 54, or 91 to 93 one or more mutations that reduce the binding by pre-existing antibodies.

11. The polypeptide according to any one of items 1 to 10, wherein the polypeptide comprises:
(i) at least one immunoglobulin single variable domain (ISVD) which specifically binds to a serum albumin protein as defined in any one of items 1 to 10; and
(ii) at least one human serum albumin protein.

12. The polypeptide according to any one of items 1 to 10, wherein the polypeptide comprises:
(i) at least one immunoglobulin single variable domain (ISVD) which specifically binds to a serum albumin protein as defined in any one of items 1 to 10; and
(ii) at least one further moiety specifically binding to a serum albumin protein,
wherein the (ii) at least one further moiety specifically binding to a serum albumin protein is selected from an antibody or fragment thereof, a single chain variable fragment (scFv), preferably an immunoglobulin single variable domain (ISVD), a DARP-in, an affibody, an Affitin and an albumin binding domain (ABD).

13. The polypeptide according to any one of items 1 to 10, wherein the (ii) at least one further moiety specifically binding to a serum albumin protein is an ISVD, preferably wherein the ISVD is derived from V_{H} or V_{H}H, even more preferably wherein the ISVD is a single domain antibody (dAb).

14. The polypeptide according to item 13, wherein the (ii) at least one further moiety specifically binding to a serum albumin protein is an ISVD comprising three complementarity determining regions (CDR1 to CDR3, respectively), wherein:
a) CDR1 comprises the amino acid sequence of SEQ ID NO: 1 or has 3, 2 or 1 amino acid difference(s) with SEQ ID NO: 1 or CDR1 comprises the amino acid sequence of SEQ ID NO: 22 or has 3, 2 or 1 amino acid difference(s) with SEQ ID NO: 22
b) CDR2 comprises the amino acid sequence of SEQ ID NO: 2 or has 3, 2 or 1 amino acid difference(s) with SEQ ID NO: 2; and
c) CDR3 comprises the amino acid sequence of SEQ ID NO: 3 or has 3, 2 or 1 amino acid difference(s) with SEQ ID NO: 3,
and/or
a) CDR1 comprises the amino acid sequence of SEQ ID NO: 36 or has 3, 2 or 1 amino acid difference(s) with SEQ ID NO: 36;
b) CDR2 comprises the amino acid sequence of SEQ ID NO: 37 or has 3, 2 or 1 amino acid difference(s) with SEQ ID NO: 37; and
c) CDR3 comprises the amino acid sequence of SEQ ID NO: 38 or has 3, 2 or 1 amino acid difference(s) with SEQ ID NO: 38,
and/or
a) CDR1 comprises the amino acid sequence of SEQ ID NO: 55 or has 3, 2 or 1 amino acid difference(s) with SEQ ID NO: 55;
b) CDR2 comprises the amino acid sequence of SEQ ID NO: 56 or has 3, 2 or 1 amino acid difference(s) with SEQ ID NO: 56; and
c) CDR3 comprises the amino acid sequence of SEQ ID NO: 57 or has 3, 2 or 1 amino acid difference(s) with SEQ ID NO: 57,
   wherein the CDR sequences are determined according to AbM numbering.

15. The polypeptide according to item 14, wherein the (ii) at least one further moiety specifically binding to a serum albumin protein is an ISVD comprising a CDR1 that comprises the amino acid sequence of SEQ ID NO: 1 or 22, a CDR2 that comprises the amino acid sequence of SEQ ID NO: 2; and a CDR3 that comprises the amino acid sequence of SEQ ID NO: 3, wherein the CDR sequences are determined according to AbM numbering.

16. The polypeptide according to any one of items 14 or 15, wherein the (ii) at least one further moiety specifically binding to a serum albumin protein is an ISVD comprising a CDR1 that comprises the amino acid sequence of SEQ ID NO: 36, a CDR2 that comprises the amino acid sequence of SEQ ID NO: 37; and a CDR3 that comprises the amino acid sequence of SEQ ID NO: 38, wherein the CDR sequences are determined according to AbM numbering, or wherein the (ii) at least one further moiety specifically binding to a serum albumin protein is an ISVD comprising a CDR1 that comprises the amino acid sequence of SEQ ID NO: 55, a CDR2 that comprises the amino acid sequence of SEQ ID NO: 56; and a CDR3 that comprises the amino acid sequence of SEQ ID NO: 57, wherein the CDR sequences are determined according to AbM numbering.

17. The polypeptide according to any one of items 14 to 16, wherein the (ii) at least one further moiety specifically binding to a serum albumin protein is an ISVD which has:
a) a degree of sequence identity with any one of the sequences as defined in SEQ ID NO's: 4 to 21, 54, or 91 to 93 of at least 85%, preferably at least 90%, more preferably at least 95%, in which any C-terminal extension that may be present as well as the CDRs are not taken into account for determining the degree of sequence identity; and/or
b) no more than 7, preferably no more than 5, such as only 3, 2 or 1 amino acid differences with any one of the sequences as defined in SEQ ID NO's: 4 to 21, 54, or 91 to 93.

18. The polypeptide according to according to any one of items 14 to 17, wherein the ((ii) at least one further moiety specifically binding to a serum albumin protein is an ISVD which has a sequence that is chosen from SEQ ID NO's: 4 to 21, 54, or 91 to 93.

19. The polypeptide according to any one of items 1 to 5, wherein the amino acid sequence of the (ii) at least one further moiety specifically binding to a serum albumin protein is an ISVD which has a sequence identity of more than 90% with SEQ ID NO: 4 or SEQ ID NO.: 18.

20. The polypeptide according to any one of items 14 to 19, wherein the (ii) at least one further moiety specifically binding to a serum albumin protein is an ISVD which comprises or consists of the amino acid sequence of Alb-23002 (SEQ ID NO: 4).

21. The polypeptide according to any one of items 14 to 20, wherein the (ii) at least one further moiety specifically binding to a serum albumin protein is an ISVD which specifically binds to serum albumin with a dissociation constant (K_{D}) of between 10⁻⁶ M and 10⁻¹¹ M or less, as determined using Proteon, Kinexa, BLI or SPR.

22. The polypeptide according to any one of items 14 to 21, wherein the ((ii) at least one further moiety specifically binding to a serum albumin protein is an ISVD which contains, compared to any of the sequences of SEQ ID NO's: 4 to 21, 54, or 91 to 93, one or more mutations that reduce the binding by pre-existing antibodies.

23. The polypeptide according to any one of items 1 to 22, wherein the (i) at least one ISVDs specifically binding to a serum albumin protein and (ii) the at least one further moiety specifically binding to a serum albumin protein or at least one human serum albumin protein are suitably linked to each other either directly or via one or more suitable linkers or spacers.

24. The polypeptide according to item 23, wherein the (i) at least one ISVDs specifically binding to a serum albumin protein and (ii) the at least one further moiety specifically binding to a serum albumin protein or at least one human serum albumin protein are suitably linked to each other via a linker selected from SEQ ID NO.: 41 to 53, preferably selected from SEQ ID NO.: 45 and 52.

25. A fusion protein comprising the polypeptide as defined in any of items 1 to 24, wherein the fusion protein further comprises one or more (such as one or two) other amino acid sequences, (binding) domains, binding units or other moieties or chemical entities.

26. The fusion protein according to item 25, wherein the polypeptide as defined in any one of items 1-24 is either directly linked to the at least one other amino acid sequences, (binding) domains, binding units or other moieties or chemical entities or is linked to the at least one other amino acid sequences, (binding) domains, binding units or other moieties or chemical entities via a linker or spacer.

27. The fusion protein according to any one of items 25 to 26, wherein the fusion protein comprises the polypeptide as defined in any one of items 1 to 24 and at least one therapeutic and/or targeting moiety.

28. The fusion protein according to any one of items 25 to 27, wherein the at least one other amino acid sequences, (binding) domains, binding units or other moieties or chemical entities comprises an immunoglobulin sequence or a fragment thereof, a single chain variable fragment (scFv), preferably an immunoglobulin single variable domain (ISVD), such as a (single) domain antibody, a V_{HH}, a humanized V_{HH} or a camelized V_{H}.

29. The fusion protein according to any of items 27 to 28, wherein the therapeutic and/or targeting moiety comprises at least one ISVD specifically binding to a therapeutic target.

30. The fusion protein according to item 29, wherein the therapeutic and/or targeting moiety comprises a single chain variable fragment (scFv), preferably an immunoglobulin single variable domain (ISVD), such as a (single) domain antibody, a V_{HH}, a humanized V_{HH} or a camelized V_{H}.

31. The fusion protein according to any one of items 25 to 30, wherein the fusion protein further comprises one further ISVD specifically binding to (human) serum albumin.

32. The polypeptide according to any of items 1 to 24 or the fusion protein according to any of items 25 to 30, wherein the polypeptide and/orthe fusion protein has a serum half-life that is at least 2 times, such as at least 5 times, preferably at least 10 times or more than 20 times, such as more than 50 times, more than 100 times, more than 500 times, preferably more than 1000 times greater than the half-life of the corresponding other moiety per se.

33. The polypeptide according to any of items 1 to 24 or the fusion protein according to any of items 25 to 30, wherein the polypeptide and/or fusion protein has a serum half-life in humans that is at least 5%, such as at least 10%, at least 25%, at least 50%, at least 100%, at least 200%, at least 300%, at least 400% or at least 500% of the half-life of serum albumin in humans.

34. A composition comprising the polypeptide as defined in any one of items 1 to 24 and/or a fusion protein as defined in any one of items 25 to 30.

35. The composition according to item 34, wherein the composition is a pharmaceutical composition. 36. Method for producing the polypeptide as defined in any one of items 1 to 24 and/or the fusion protein as defined in any one of items 25 to 30, wherein the method comprises the steps of:
expressing, in a suitable host cell or host organism or in another suitable expression system, a nucleic acid sequence encoding the polypeptide as defined in any one of items 1 to 24 and/or the fusion protein as defined in any one of items 25 to 30; optionally followed by: isolating and/or purifying the polypeptide as defined in any one of items 1 to 24 and/or the fusion protein as defined in any one of items 25 to 30.

37. Nucleic acid or nucleic acid sequence encoding the polypeptide as defined in any one of items 1 to 24 and/or the fusion protein as defined in any one of items 25 to 30.

38. Vector comprising a nucleic acid or nucleic acid sequence as defined in item 37.

39. Non-human host or host cell comprising a vector comprising a nucleic acid sequence as defined in item 38 or expressing a nucleic acid sequence as defined in item 37.

40. The polypeptide according to any one of items 1 to 24, the fusion protein as defined in any one of items 25 to 30, and/or the composition according to any one of items 34 to 35 for use as a medicament.

41. Kit comprising the polypeptide according to any of items 1 to 24, the fusion protein as defined in any one of items 25 to 30, a nucleic acid or nucleic acid sequence according to item 37, a vector according to item 38, or a host cell according to item 39.

42. The polypeptide according to any one of items 1 to 24, wherein the polypeptide comprises or consist of a sequence selected from SEQ ID NO.: 74-81, 87 and 147-154, or a sequence which comprises or consists of a sequence with at least 90%, such as at least 95%, or at least 97%, or at least 99% sequence identity with a sequence selected from SEQ ID NO.: 74-81, 87 and 147-154.

43. The fusion protein as defined in any one of items 25 to 30, wherein the fusion protein comprises or consists of a sequence selected from SEQ ID NO.: 82-86, or a sequence which comprises or consists of a sequence with at least 90%, such as at least 95%, or at least 97%, or at least 99% sequence identity with a sequence selected from SEQ ID NO.: 82-86.

1. A polypeptide comprising:
(i) at least one immunoglobulin single variable domain (ISVD) which specifically binds to a serum albumin protein; and (ii) at least one further moiety specifically binding to a serum albumin protein or at least one human serum albumin protein,
wherein said (i) at least one ISVD essentially consists of 4 framework regions (FR1 to FR4, respectively) and three complementarity determining regions (CDR1 to CDR3, respectively), wherein:
CDR1 comprises the amino acid sequence of SEQ ID NO: 1 or has 3, 2 or 1 amino acid difference(s) with SEQ ID NO: 1 or CDR1 comprises the amino acid sequence of SEQ ID NO: 22 or has 3, 2 or 1 amino acid difference(s) with SEQ ID NO: 22
CDR2 comprises the amino acid sequence of SEQ ID NO: 2 or has 3, 2 or 1 amino acid difference(s) with SEQ ID NO: 2; and
CDR3 comprises the amino acid sequence of SEQ ID NO: 3 or has 3, 2 or 1 amino acid difference(s) with SEQ ID NO: 3,
and/or
CDR1 comprises the amino acid sequence of SEQ ID NO: 36 or has 3, 2 or 1 amino acid difference(s) with SEQ ID NO: 36;
CDR2 comprises the amino acid sequence of SEQ ID NO: 37 or has 3, 2 or 1 amino acid difference(s) with SEQ ID NO: 37; and
CDR3 comprises the amino acid sequence of SEQ ID NO: 38 or has 3, 2 or 1 amino acid difference(s) with SEQ ID NO: 38,
and/or
CDR1 comprises the amino acid sequence of SEQ ID NO: 55 or has 3, 2 or 1 amino acid difference(s) with SEQ ID NO: 55;
CDR2 comprises the amino acid sequence of SEQ ID NO: 56 or has 3, 2 or 1 amino acid difference(s) with SEQ ID NO: 56; and
CDR3 comprises the amino acid sequence of SEQ ID NO: 57 or has 3, 2 or 1 amino acid difference(s) with SEQ ID NO: 57
wherein the CDR sequences are determined according to AbM numbering.

2. The polypeptide according to claim 1, wherein the (i) at least one ISVD specifically binding to a serum albumin protein comprises:
a CDR1 that comprises the amino acid sequence of SEQ ID NO: 1 or 22, a CDR2 that comprises the amino acid sequence of SEQ ID NO: 2; and a CDR3 that comprises the amino acid sequence of SEQ ID NO: 3; or
a CDR1 that comprises the amino acid sequence of SEQ ID NO: 36, a CDR2 that comprises the amino acid sequence of SEQ ID NO: 37; and a CDR3 that comprises the amino acid sequence of SEQ ID NO: 38; or
a CDR1 that comprises the amino acid sequence of SEQ ID NO: 55, a CDR2 that comprises the amino acid sequence of SEQ ID NO: 56; and a CDR3 that comprises the amino acid sequence of SEQ ID NO: 57;
wherein the CDR sequences are determined according to AbM numbering.

3. The polypeptide according to claim 1, wherein the (i) at least one ISVD specifically binding to a serum albumin protein has:
a degree of sequence identity with any one of the sequences as defined in SEQ ID NO's: 4 to 21, 54, or 91 to 93 of at least 85%, in which any C-terminal extension that may be present as well as the CDRs are not taken into account for determining the degree of sequence identity; and/or
no more than 7 amino acid differences with any one of the sequences as defined in SEQ ID NO's: 4 to 21, 54, or 91 to 93.

4. The polypeptide according to claim 1, wherein the (i) at least one ISVD comprised therein is derived from a V_{H}, a humanized V_{H}, a human V_{H}, a V_{HH}, a humanized V_{HH} or a camelized V_{H}.

5. The polypeptide according to claim 1, wherein the polypeptide comprises
at least one human serum albumin protein.

6. The polypeptide according to claim 1, wherein the polypeptide comprises
at least one further moiety specifically binding to a serum albumin protein,
wherein the (ii) at least one further moiety specifically binding to a serum albumin protein is selected from the group consisting of an antibody or fragment thereof, a single chain variable fragment (scFv), a DARP-in, an affibody, an Affitin and an albumin binding domain (ABD).

7. The polypeptide according to claim 1, wherein the polypeptide comprises at least one further moiety specifically binding to a serum albumin protein, wherein the (ii) at least one further moiety specifically binding to a serum albumin protein is an ISVD comprising three complementarity determining regions (CDR1 to CDR3, respectively), wherein:
CDR1 comprises the amino acid sequence of SEQ ID NO: 1 or has 3, 2 or 1 amino acid difference(s) with SEQ ID NO: 1 or CDR1 comprises the amino acid sequence of SEQ ID NO: 22 or has 3, 2 or 1 amino acid difference(s) with SEQ ID NO: 22;
CDR2 comprises the amino acid sequence of SEQ ID NO: 2 or has 3, 2 or 1 amino acid difference(s) with SEQ ID NO: 2; and
CDR3 comprises the amino acid sequence of SEQ ID NO: 3 or has 3, 2 or 1 amino acid difference(s) with SEQ ID NO: 3,
and/or
CDR1 comprises the amino acid sequence of SEQ ID NO: 36 or has 3, 2 or 1 amino acid difference(s) with SEQ ID NO: 36;
CDR2 comprises the amino acid sequence of SEQ ID NO: 37 or has 3, 2 or 1 amino acid difference(s) with SEQ ID NO: 37; and
CDR3 comprises the amino acid sequence of SEQ ID NO: 38 or has 3, 2 or 1 amino acid difference(s) with SEQ ID NO: 38,
and/or
CDR1 comprises the amino acid sequence of SEQ ID NO: 55 or has 3, 2 or 1 amino acid difference(s) with SEQ ID NO: 55;
CDR2 comprises the amino acid sequence of SEQ ID NO: 56 or has 3, 2 or 1 amino acid difference(s) with SEQ ID NO: 56; and
CDR3 comprises the amino acid sequence of SEQ ID NO: 57 or has 3, 2 or 1 amino acid difference(s) with SEQ ID NO: 57,
wherein the CDR sequences are determined according to AbM numbering.

8. The polypeptide according to claim 7, wherein the (ii) at least one further moiety specifically binding to a serum albumin protein is an ISVD which has:
a degree of sequence identity with any one of the sequences as defined in SEQ ID NO's: 4 to 21, 54, or 91 to 93 of at least 85%, in which any C-terminal extension that may be present as well as the CDRs are not taken into account for determining the degree of sequence identity;
and/or
no more than 7 amino acid differences with any one of the sequences as defined in SEQ ID NO's: 4 to 21, 54, or 91 to 93.

9. A fusion protein comprising the polypeptide as defined in claim 1, wherein the fusion protein further comprises one or more other amino acid sequences, domains, binding units or other moieties or chemical entities.

10. The fusion protein according to claim 9, wherein the fusion protein comprises at least one therapeutic and/or targeting moiety.

11. A composition comprising the polypeptide as defined in claim 1.

12. A nucleic acid or nucleic acid sequence encoding the polypeptide as defined in claim 1.

13. A vector comprising a nucleic acid or nucleic acid sequence as defined in claim 12.

14. A non-human host or host cell comprising a nucleic acid sequence as defined in claim 12.

15. A method of treatment comprising administering a polypeptide according to claim 1 to a subject in need thereof.

16. A method of producing a polypeptide comprising expressing a nucleic acid sequence as defined in claim 12 in a suitable host cell.

17. The polypeptide according to claim 3, wherein the (i) at least one ISVD specifically binding to a serum albumin protein comprises any one of the sequences as defined in SEQ ID NO's: 4 to 21, 54, or 91 to 93.

18. The polypeptide according to claim 6, wherein the polypeptide comprises at least one further moiety specifically binding to a serum albumin protein, wherein the (ii) at least one further moiety specifically binding to a serum albumin protein is an immunoglobulin single variable domain (ISVD).

19. The polypeptide according to claim 8, wherein the (ii) at least one further moiety specifically binding to a serum albumin protein is an ISVD which comprises any one of the sequences as defined in SEQ ID NO's: 4 to 21, 54, or 91 to 93.

20. The fusion protein of claim 10, wherein the therapeutic and/or targeting moiety comprises at least one ISVD specifically binding to a therapeutic target or wherein the fusion protein further comprises one further ISVD specifically binding to serum albumin.


SEQUENCE LISTING (154 sequences)

SEQ ID NO: 1 - protein, 10 aa, synthetic construct
  REGION 1..10: note = CDR1 Alb 23 AbM
GFTFRSFGMS

SEQ ID NO: 2 - protein, 10 aa, synthetic construct
  REGION 1..10: note = CDR2 Alb 23 AbM
SISGSGSDTL

SEQ ID NO: 3 - protein, 6 aa, synthetic construct
  REGION 1..6: note = CDR3 Alb 23 AbM
GGSLSR

SEQ ID NO: 4 - protein, 115 aa, synthetic construct
  REGION 1..115: note = Alb23002
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 5 - protein, 115 aa, synthetic construct [sequence omitted]

SEQ ID NO: 6 - protein, 115 aa, synthetic construct [sequence omitted]

SEQ ID NO: 7 - protein, 116 aa, synthetic construct
  REGION 1..116: note = Alb129
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSSA

SEQ ID NO: 8 - protein, 116 aa, synthetic construct
  REGION 1..116: note = Alb132
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSSA

SEQ ID NO: 9 - protein, 115 aa, synthetic construct [sequence omitted]

SEQ ID NO: 10 - protein, 116 aa, synthetic construct
  REGION 1..116: note = Alb11 (S112K)-A
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSSA

SEQ ID NO: 11 - protein, 115 aa, synthetic construct
  REGION 1..115: note = Alb82
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 12 - protein, 116 aa, synthetic construct
  REGION 1..116: note = Alb82-A
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 13 - protein, 117 aa, synthetic construct
  REGION 1..117: note = Alb82-AA
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAA

SEQ ID NO: 14 - protein, 118 aa, synthetic construct
  REGION 1..118: note = Alb82-AAA
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAAA

SEQ ID NO: 15 - protein, 116 aa, synthetic construct
  REGION 1..116: note = Alb82-G
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSG

SEQ ID NO: 16 - protein, 117 aa, synthetic construct
  REGION 1..117: note = Alb82-GG
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGG

SEQ ID NO: 17 - protein, 118 aa, synthetic construct
  REGION 1..118: note = Alb82-GGG
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGGG

SEQ ID NO: 18 - protein, 116 aa, synthetic construct
  REGION 1..116: note = Alb223
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 19 - protein, 115 aa, synthetic construct
  REGION 1..115: note = Alb23002(E1D)
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 20 - protein, 124 aa, synthetic construct
  REGION 1..124: note = T023500029
EVQLVESGGGVVQPGDSLRLSCAASGGTFSTYVMGWFRQAPGKEREFVSAISQNSIHTYYANSVKGRFTISRDNSKNTVYLQLNSLRPEDTALYYCAASRFTSWYTADYEYDYWGQGTLVTVSS

SEQ ID NO: 21 - protein, 123 aa, synthetic construct
  REGION 1..123: note = AlbX00001
EVQLVESGGGVVQPGGSLRLSCAASGLTFSSYAMGWFRQAPGKERERVVSISRGGGYTYYADSVKGRFTISRDNSENTVYLQMNSLRPEDTALYYCAAARYWATGSEYEFDYWGQGTLVTVSS

SEQ ID NO: 22 - protein, 10 aa, synthetic construct
GFTFSSFGMS

SEQ ID NO: 23 - protein, 25 aa, synthetic construct
EVQLVESGGGVVQPGGSLRLSCAAS

SEQ ID NO: 24 - protein, 14 aa, synthetic construct
WVRQAPGKGPEWVS

SEQ ID NO: 25 - protein, 39 aa, synthetic construct
YADSVKGRFTISRDNSKNTLYLQMNSLRPEDTALYYCTI

SEQ ID NO: 26 - protein, 11 aa, synthetic construct
SSQGTLVTVSS

SEQ ID NO: 27 - protein, 5 aa, synthetic construct
SFGMS

SEQ ID NO: 28 - protein, 17 aa, synthetic construct
SISGSGSDTLYADSVKG

SEQ ID NO: 29 - skipped

SEQ ID NO: 30 - protein, 30 aa, synthetic construct
EVQLVESGGGVVQPGGSLRLSCAASGFTFR

SEQ ID NO: 31 - skipped

SEQ ID NO: 32 - protein, 32 aa, synthetic construct
RFTISRDNSKNTLYLQMNSLRPEDTALYYCTI

SEQ ID NO: 33 - skipped

SEQ ID NO: 34 - protein, 5 aa, synthetic construct
TYVMG

SEQ ID NO: 35 - protein, 17 aa, synthetic construct
AISQNSIHTYYANSVKG

SEQ ID NO: 36 - protein, 10 aa, synthetic construct
GGTFSTYVMG

SEQ ID NO: 37 - protein, 10 aa, synthetic construct
AISQNSIHTY

SEQ ID NO: 38 - protein, 15 aa, synthetic construct
SRFTSWYTADYEYDY

SEQ ID NO: 39 - protein, 585 aa, Homo sapiens
  REGION 1..585: note = Human serum albumin (1)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 40 - protein, 585 aa, Homo sapiens [sequence omitted]

SEQ ID NO: 41 - skipped

SEQ ID NO: 42 - protein, 5 aa, synthetic construct
  REGION 1..5: note = 5GS linker
GGGGS

SEQ ID NO: 43 - protein, 7 aa, synthetic construct
  REGION 1..7: note = 7GS linker
SGGSGGS

SEQ ID NO: 44 - protein, 8 aa, synthetic construct
  REGION 1..8: note = 8GS linker
GGGGSGGS

SEQ ID NO: 45 - protein, 9 aa, synthetic construct
  REGION 1..9: note = 9GS linker
GGGGSGGGS

SEQ ID NO: 46 - protein, 10 aa, synthetic construct
  REGION 1..10: note = 10GS linker
GGGGSGGGGS

SEQ ID NO: 47 - protein, 15 aa, synthetic construct
  REGION 1..15: note = 15GS linker
GGGGSGGGGSGGGGS

SEQ ID NO: 48 - protein, 18 aa, synthetic construct
  REGION 1..18: note = 18GS linker
GGGGSGGGGSGGGGSGGS

SEQ ID NO: 49 - protein, 20 aa, synthetic construct
  REGION 1..20: note = 20GS linker
GGGGSGGGGSGGGGSGGGGS

SEQ ID NO: 50 - protein, 25 aa, synthetic construct
  REGION 1..25: note = 25GS linker
GGGGSGGGGSGGGGSGGGGSGGGGS

SEQ ID NO: 51 - protein, 30 aa, synthetic construct
  REGION 1..30: note = 30GS linker
GGGGSGGGGSGGGGSGGGGSGGGGSGGGGS

SEQ ID NO: 52 - protein, 35 aa, synthetic construct
  REGION 1..35: note = 35GS linker
GGGGSGGGGSGGGGSGGGGSGGGGSGGGGSGGGGS

SEQ ID NO: 53 - protein, 40 aa, synthetic construct
  REGION 1..40: note = 40GS linker
GGGGSGGGGSGGGGSGGGGSGGGGSGGGGSGGGGSGGGGS

SEQ ID NO: 54 - protein, 123 aa, synthetic construct
  REGION 1..123: note = ALBX00002
EVQLVESGGGVVQPGGSLRLSCAASGLTFSSYAMGWFRQAPGKERERVVSISRGGGYTYYADSVKGRFTISRDNSKNTVYLQMNSLRPEDTALYYCAAARYWATGSEYEFDYWGQGTLVTVSS

SEQ ID NO: 55 - protein, 10 aa, synthetic construct
  REGION 1..10: note = ALBX00002 CDR1 (AbM)
GLTFSSYAMG

SEQ ID NO: 56 - protein, 10 aa, synthetic construct
  REGION 1..10: note = ALBX00002 CDR2 (AbM)
SISRGGGYTY

SEQ ID NO: 57 - protein, 14 aa, synthetic construct
  REGION 1..12: note = ALBX00002 CDR3 (AbM and Kabat)
ARYWATGSEYEFDY

SEQ ID NO: 58 - protein, 5 aa, synthetic construct
  REGION 1..5: note = ALBX00002 CDR1 (Kabat)
SYAMG

SEQ ID NO: 59 - protein, 17 aa, synthetic construct
  REGION 1..17: note = ALBX00002 CDR2 (Kabat)
SISRGGGYTYYADSVKG

SEQ ID NO: 60 - protein, 25 aa, synthetic construct
  REGION 1..25: note = T023500029 FR1 (Abm)
EVQLVESGGGVVQPGDSLRLSCAAS

SEQ ID NO: 61 - protein, 14 aa, synthetic construct
WFRQAPGKEREFVS

SEQ ID NO: 62 - protein, 39 aa, synthetic construct
  REGION 1..14: note = T023500029 FR2 (Abm)
YANSVKGRFTISRDNSKNTVYLQLNSLRPEDTALYYCAA

SEQ ID NO: 63 - protein, 11 aa, synthetic construct
  REGION 1..11: note = T023500029 FR4 (Abm and Kabat)
WGQGTLVTVSS

SEQ ID NO: 64 - protein, 30 aa, synthetic construct
  REGION 1..30: note = T023500029 FR1 (Kabat)
EVQLVESGGGVVQPGDSLRLSCAASGGTFS

SEQ ID NO: 65 - protein, 14 aa, synthetic construct
  REGION 1..14: note = T023500029 FR2 (Kabat)
WFRQAPGKEREFVS

SEQ ID NO: 66 - protein, 32 aa, synthetic construct
  REGION 1..32: note = T023500029 FR3 (Kabat)
RFTISRDNSKNTVYLQLNSLRPEDTALYYCAA

SEQ ID NO: 67 - skipped

SEQ ID NO: 68 - protein, 14 aa, synthetic construct
  REGION 1..14: note = ALBX00002 FR2 (AbM)
WFRQAPGKERERVV

SEQ ID NO: 69 - protein, 39 aa, synthetic construct
  REGION 1..39: note = ALBX00002 FR3 (AbM)
YADSVKGRFTISRDNSKNTVYLQMNSLRPEDTALYYCAA

SEQ ID NO: 70 - protein, 11 aa, synthetic construct
  REGION 1..11: note = ALBX00002 FR4 (AbM and Kabat)
WGQGTLVTVSS

SEQ ID NO: 71 - protein, 30 aa, synthetic construct
  REGION 1..30: note = ALBX00002 FR1 (Kabat)
EVQLVESGGGVVQPGGSLRLSCAASGLTFS

SEQ ID NO: 72 - skipped

SEQ ID NO: 73 - protein, 32 aa, synthetic construct
  REGION 1..32: note = ALBX00002 FR3 (Kabat)
RFTISRDNSKNTVYLQMNSLRPEDTALYYCAA

SEQ ID NO: 74 - protein, 266 aa, synthetic construct
  REGION 1..266: note = HSA006A06-35GS-HSA006A06-A
EVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGGSGGGGSGGGGSGGGGSGGGGSGGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSA

SEQ ID NO: 75 - protein, 266 aa, synthetic construct
  REGION 1..266: note = ALB11002-35GS-ALB11002-A
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGGGGSGGGGSGGGGSGGGGSGGGGSGGGGSGGGGSEVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 76 - protein, 266 aa, synthetic construct
  REGION 1..266: note = HSA006A06-35GS-ALB23002-A
EVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 77 - protein, 274 aa, synthetic construct
  REGION 1..274: note = ALBX00002-35GS-ALB23002-A
EVQLVESGGGVVQPGGSLRLSCAASGLTFSSYAMGWFRQAPGKERERVVSISRGGGYTYYADSVKGRFTISRDNSKNTVYLQMNSLRPEDTALYYCAAARYWATGSEYEFDYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 78 - protein, 275 aa, synthetic construct
  REGION 1..275: note = T0235002C06(L11V,T14P,D74S,K83R,V89L)-35GS-ALB23002 -A
EVQLVESGGGVVQPGDSLRLSCAASGGTFSTYVMGWFRQAPGKEREFVSAISQNSIHTYYANSVKGRFTISRDNSKNTVYLQLNSLRPEDTALYYCAASRFTSWYTADYEYDYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 79 - protein, 275 aa, synthetic construct
  REGION 1..275: note = DARPIN-35GS-ALB23002-A
DLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAAGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 80 - protein, 215 aa, synthetic construct
  REGION 1..215: note = Nanofitin-35GS-ALB23002-A
VKVKFWPRGEEKVVDTSKIAWVLRADKTVMFKYDDNGKKGYGVVLEKDAPKELLDMLARAEREKGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 81 - protein, 196 aa, synthetic construct
  REGION 1..196: note = ABD-35GS-ALB23002-A
LKEAKEKAIEELKKAGITSDYYFDLINKAKTVEGVNALKDEILKAGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 82 - protein, 416 aa, synthetic construct
  REGION 1..416: note = ALB23002-35GS-ALB23002-35GS-ALB23002-A
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 83 - protein, 433 aa, synthetic construct
  REGION 1..433: note = ALBX00002-35GS-T0235002C06(L11V,T14P,D74S,K83R,V89L )-35GS-ALB23002-A
EVQLVESGGGVVQPGGSLRLSCAASGLTFSSYAMGWFRQAPGKERERVVSISRGGGYTYYADSVKGRFTISRDNSKNTVYLQMNSLRPEDTALYYCAAARYWATGSEYEFDYWGQGTLVTVSSGGGGSGGGGSGGGGSGGGGSGGGGSGGGGSGGGGSEVQLVESGGGVVQPGDSLRLSCAASGGTFSTYVMGWFRQAPGKEREFVSAISQNSIHTYYANSVKGRFTISRDNSKNTVYLQLNSLRPEDTALYYCAASRFTSWYTADYEYDYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 84 - protein, 425 aa, synthetic construct
  REGION 1..425: note = DARPIN-35GS-ALB23002-35GS-ALB23002-A
DLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAAGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 85 - protein, 566 aa, synthetic construct
  REGION 1..566: note = ALB23002-35GS-ALB23002-35GS-ALB23002-35GS-ALB23002- A
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 86 - protein, 583 aa, synthetic construct
  REGION 1..583: note = ALBX00002-35GS-T0235002C06(L11V,T14P,D74S,K83R,V89L )-35GS-ALB23002-35GS-ALB23002-A
EVQLVESGGGVVQPGGSLRLSCAASGLTFSSYAMGWFRQAPGKERERVVSISRGGGYTYYADSVKGRFTISRDNSKNTVYLQMNSLRPEDTALYYCAAARYWATGSEYEFDYWGQGTLVTVSSGGGGSGGGGSGGGGSGGGGSGGGGSGGGGSGGGGSEVQLVESGGGVVQPGDSLRLSCAASGGTFSTYVMGWFRQAPGKEREFVSAISQNSIHTYYANSVKGRFTISRDNSKNTVYLQLNSLRPEDTALYYCAASRFTSWYTADYEYDYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 87 - protein, 1205 aa, synthetic construct
  REGION 1..1205: note = HSA(25-609)-35GS-HSA(25-609)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSGGGGSGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 88 - protein, 124 aa, synthetic construct
  REGION 1..124: note = Darpin
DLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 89 - protein, 64 aa, synthetic construct
  REGION 1..64: note = Affitin (Nanofitin)
VKVKFWPRGEEKVVDTSKIAWVLRADKTVMFKYDDNGKKGYGVVLEKDAPKELLDMLARAEREK

SEQ ID NO: 90 - protein, 45 aa, synthetic construct
  REGION 1..45: note = ABD
LKEAKEKAIEELKKAGITSDYYFDLINKAKTVEGVNALKDEILKA

SEQ ID NO: 91 - protein, 115 aa, synthetic construct [sequence omitted]

SEQ ID NO: 92 - protein, 116 aa, synthetic construct
  REGION 1..116: note = HSA006A06-A
EVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSA

SEQ ID NO: 93 - protein, 115 aa, synthetic construct [sequence omitted]

SEQ ID NO: 94 - protein, 5 aa, synthetic construct
VTVSS

SEQ ID NO: 95 - protein, 5 aa, synthetic construct
  REGION 1..5: note = Hallmark Residues at positions 43-47
KEREL

SEQ ID NO: 96 - protein, 5 aa, synthetic construct
  REGION 1..5: note = Hallmark Residues at positions 43-47
KEREF

SEQ ID NO: 97 - protein, 5 aa, synthetic construct
KQREL

SEQ ID NO: 98 - protein, 5 aa, synthetic construct
  REGION 1..5: note = Hallmark Residues at positions 43-47
KQREF

SEQ ID NO: 99 - protein, 5 aa, synthetic construct
  REGION 1..5: note = Hallmark Residues at positions 43-47
KEREG

SEQ ID NO: 100 - protein, 5 aa, synthetic construct
  REGION 1..5: note = Hallmark Residues at positions 43-47
KQREW

SEQ ID NO: 101 - protein, 5 aa, synthetic construct
  REGION 1..5: note = Hallmark Residues positions 43-47
KQREG

SEQ ID NO: 102 - protein, 5 aa, synthetic construct
TEREL

SEQ ID NO: 103 - protein, 5 aa, synthetic construct
TQREL

SEQ ID NO: 104 - protein, 5 aa, synthetic construct
  REGION 1..5: note = Hallmark Residues positions 43-47
  VARIANT 5: note = X can be L or R
KECEX

SEQ ID NO: 105 - protein, 5 aa, synthetic construct
  REGION 1..5: note = Hallmark Residues positions 43-47
KQCEL

SEQ ID NO: 106 - protein, 5 aa, synthetic construct
  REGION 1..5: note = Hallmark Residues positions 43-47
REREG

SEQ ID NO: 107 - protein, 5 aa, synthetic construct
  REGION 1..5: note = Hallmark Residues positions 43-47
  VARIANT 5: note = X can be L,F or W
RQREX

SEQ ID NO: 108 - protein, 5 aa, synthetic construct
  REGION 1..5: note = Hallmark Residues positions 43-47.
QEREG

SEQ ID NO: 109 - protein, 5 aa, synthetic construct
  REGION 1..5: note = Hallmark Residues positions 43-47
  VARIANT 5: note = X can be W, L or F
QQREX

SEQ ID NO: 110 - protein, 5 aa, synthetic construct
  REGION 1..5: note = KGREG
KGREG

SEQ ID NO: 111 - protein, 5 aa, synthetic construct
  REGION 1..5: note = Hallmark Residues positions 43-47
KDREV

SEQ ID NO: 112 - protein, 4 aa, synthetic construct
KERE

SEQ ID NO: 113 - protein, 4 aa, synthetic construct
KQRE

SEQ ID NO: 114 - protein, 4 aa, synthetic construct
GLEW

SEQ ID NO: 115 - protein, 4 aa, synthetic construct
TERE

SEQ ID NO: 116 - protein, 4 aa, synthetic construct
TQRE

SEQ ID NO: 117 - protein, 4 aa, synthetic construct
KECE

SEQ ID NO: 118 - protein, 5 aa, synthetic construct
KECEL

SEQ ID NO: 119 - protein, 5 aa, synthetic construct
KECER

SEQ ID NO: 120 - protein, 4 aa, synthetic construct
KQCE

SEQ ID NO: 121 - protein, 4 aa, synthetic construct
RERE

SEQ ID NO: 122 - protein, 4 aa, synthetic construct
RQRE

SEQ ID NO: 123 - protein, 5 aa, synthetic construct
RQREL

SEQ ID NO: 124 - protein, 5 aa, synthetic construct
RQREF

SEQ ID NO: 125 - protein, 5 aa, synthetic construct
RQREW

SEQ ID NO: 126 - protein, 4 aa, synthetic construct
QERE

SEQ ID NO: 127 - protein, 4 aa, synthetic construct
QQRE

SEQ ID NO: 128 - protein, 5 aa, synthetic construct
QQREW

SEQ ID NO: 129 - protein, 5 aa, synthetic construct
QQREL

SEQ ID NO: 130 - protein, 5 aa, synthetic construct
QQREF

SEQ ID NO: 131 - protein, 4 aa, synthetic construct
KGRE

SEQ ID NO: 132 - protein, 4 aa, synthetic construct
KDRE

SEQ ID NO: 133 - protein, 5 aa, synthetic construct
DECKL

SEQ ID NO: 134 - protein, 5 aa, synthetic construct
NVCEL

SEQ ID NO: 135 - protein, 4 aa, synthetic construct
GVEW

SEQ ID NO: 136 - protein, 4 aa, synthetic construct
EPEW

SEQ ID NO: 137 - protein, 4 aa, synthetic construct
GLER

SEQ ID NO: 138 - protein, 4 aa, synthetic construct
DQEW

SEQ ID NO: 139 - protein, 4 aa, synthetic construct
DLEW

SEQ ID NO: 140 - protein, 4 aa, synthetic construct
GIEW

SEQ ID NO: 141 - protein, 4 aa, synthetic construct
ELEW

SEQ ID NO: 142 - protein, 4 aa, synthetic construct
GPEW

SEQ ID NO: 143 - protein, 4 aa, synthetic construct
EWLP

SEQ ID NO: 144 - protein, 4 aa, synthetic construct
GPER

SEQ ID NO: 145 - protein, 4 aa, synthetic construct
GLER

SEQ ID NO: 146 - protein, 4 aa, synthetic construct
ELEW

SEQ ID NO: 147 - protein, 266 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 148 - protein, 240 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 149 - protein, 251 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 150 - protein, 274 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSDLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 151 - protein, 195 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSLKEAKEKAIEELKKAGITSDYYFDLINKAKTVEGVNALKDEILKA

SEQ ID NO: 152 - protein, 265 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 153 - protein, 250 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 154 - protein, 239 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS